SEQUENCE LISTING (58 sequences)

SEQ ID NO: 1 - protein, 15 aa, m13 library
  VARIANT (1): Xaa at position 1 is T, S, A, or P;
XXXXXXXXXXXXXXX

SEQ ID NO: 2 - protein, 15 aa, m13 library
  VARIANT (7): It is T, S, A, Y, F, H, W, N, L, I, V, or M.
FLHTRLXXXXXXXXX

SEQ ID NO: 3 - protein, 15 aa, m13 library
  VARIANT (9): It is  V, I, L, M, S, A, T, R, Q, or K.
FLHTRLFVXXXXXXX

SEQ ID NO: 4 - protein, 15 aa, m13 library
  VARIANT (1): It is T or P.
XXXXXXXXXXXXXXX

SEQ ID NO: 5 - protein, 15 aa, m13 library
  VARIANT (7): It is T, F, or L.
FLHTRLXXXXXXXXX

SEQ ID NO: 6 - protein, 15 aa, m13 library
  VARIANT (9): It is V, S, or R.
FLHTRLFVXXXXXXX

SEQ ID NO: 7 - protein, 14 aa, m13 library
FLHTRLFVSDWYHT

SEQ ID NO: 8 - protein, 8 aa, m13 library
FLHTRLFV

SEQ ID NO: 9 - DNA, 42 bp, m13 library
ttcctccacacccggcttttcgtgagcgattggtaccacacc

SEQ ID NO: 10 - DNA, 42 bp, m13 library
ttcctccacacccggctttttgtcagcgattggtaccacaca

SEQ ID NO: 11 - DNA, 42 bp, m13 library
ttcctgcatacccggcttttcgtgagtgattggtaccacacc

SEQ ID NO: 12 - DNA, 42 bp, m13 library
ttcctacacacccggcttttcgtctcagattggtaccacacc

SEQ ID NO: 13 - DNA, 42 bp, m13 library
ttcctccacacccggcttttcgtgtccgattggtaccacacc

SEQ ID NO: 14 - DNA, 42 bp, m13 library
ttcctccacacccggcttttcgtgagcgactggtaccacacc

SEQ ID NO: 15 - DNA, 42 bp, m13 library
tttctccacacccggcttttcgtgagcgactggtaccacacc

SEQ ID NO: 16 - DNA, 42 bp, m13 library
ttcctccacacccggcttttcgtaagcgattggtaccacacg

SEQ ID NO: 17 - DNA, 42 bp, m13 library
tttctccacacccggcttttcgtgagcgattggtaccacacc

SEQ ID NO: 18 - DNA, 42 bp, m13 library
ttcctccacacccggctattcgtgagtgattggtaccacacc

SEQ ID NO: 19 - DNA, 42 bp, m13 library
ttcctccacacccgactcttcgtgagcgattggtaccacacc

SEQ ID NO: 20 - DNA, 42 bp, m13 library
ttcctacacacccggctttttgtgagcgattggtaccacacc

SEQ ID NO: 21 - DNA, 24 bp, m13 library
ttcctccacacccggcttttcgtg

SEQ ID NO: 22 - DNA, 24 bp, m13 library
ttcctccacacccggctgttcgta

SEQ ID NO: 23 - DNA, 24 bp, m13 library
ttccttcacacccggctattcgtt

SEQ ID NO: 24 - DNA, 24 bp, m13 library
ttcctacacacccggctcttcgtc

SEQ ID NO: 25 - DNA, 24 bp, m13 library
ttcctccacacacggcttttcgtg

SEQ ID NO: 26 - DNA, 24 bp, m13 library
ttcctgcacactcggcttttcgtg

SEQ ID NO: 27 - protein, 15 aa, m13 library
TFTRVVTDVYRGRLS

SEQ ID NO: 28 - protein, 15 aa, m13 library
FLHTRLFVSDWYHTP

SEQ ID NO: 29 - protein, 15 aa, m13 library
FLHTRLFVSDWYNTP

SEQ ID NO: 30 - protein, 15 aa, m13 library
FLHTRLLFRIVSYSG

SEQ ID NO: 31 - protein, 15 aa, m13 library
FLHTRLLFRNGIILR

SEQ ID NO: 32 - protein, 15 aa, m13 library
FLHTRLFVSDGINSG

SEQ ID NO: 33 - protein, 15 aa, m13 library
SGRGFCCWSNDSALS

SEQ ID NO: 34 - protein, 15 aa, Artificial Sequence
  Description of Artificial Sequence Random 15-mers in phage; not isolated from any particular organism.
TRLFRVPVFRLGDFW

SEQ ID NO: 35 - protein, 15 aa, Artificial Sequence
  Description of Artificial Sequence Random 15-mers in phage; not isolated from any particular organism.
TRLFRFLSSVWGLLA

SEQ ID NO: 36 - protein, 15 aa, Artificial Sequence
  Description of Artificial Sequence Random 15-mers in phage; not isolated from any particular organism.
TRLFRVPVLPSGVTS

SEQ ID NO: 37 - protein, 16 aa, Artificial Sequence
  Description of Artificial Sequence Random 15-mers in phage; not isolated from any particular organism.
SLAPYSLRIFVLFGGA

SEQ ID NO: 38 - protein, 17 aa, Artificial Sequence
  Description of Artificial Sequence Random 15-mers in phage; not isolated from any particular organism.
SLARSFHAYFRHTLVGP

SEQ ID NO: 39 - protein, 6 aa, Artificial Sequence
  Description of Artificial Sequence A consensus sequence found in eight clones.
TRLFRX

SEQ ID NO: 40 - protein, 6 aa, Artificial Sequence
  Description of Artificial Sequence Consensus which is conserved universally and compressed in many of the L2-412 and HNK-1 binders.
TRLFXV

SEQ ID NO: 41 - protein, 4 aa, Artificial Sequence
  Description of Artificial Sequence Consensus which is conserved universally and compressed in many of the L2-412 and HNK-1 binders.
TRLF

SEQ ID NO: 42 - DNA, 18 bp, Artificial Sequence
  Description of Artificial SequenceExample of DNA encoding Sequence ID No. 39.
acccgtctttttcggttc

SEQ ID NO: 43 - DNA, 18 bp, Artificial Sequence
  Description of Artificial SequenceExample of DNA encoding Sequence ID No. 39.
acacgcctcttccgagtt

SEQ ID NO: 44 - DNA, 18 bp, Artificial Sequence
  Description of Artificial SequenceExample of DNA encoding Sequence ID No. 39.
acgcgactatttcgtgta

SEQ ID NO: 45 - DNA, 18 bp, Artificial Sequence
  Description of Artificial Sequence Example of DNA encoding Sequence ID No. 40.
acccgccttttccgggtc

SEQ ID NO: 46 - DNA, 15 bp, Artificial Sequence
  Description of Artificial Sequence Example of DNA encoding Sequence ID No. 40.
acgcgcctcttcgta

SEQ ID NO: 47 - DNA, 15 bp, Artificial Sequence
  Description of Artificial Sequence Example of DNA encoding Sequence ID No. 40.
acacgactatttgta

SEQ ID NO: 48 - DNA, 12 bp, Artificial Sequence
  Description of Artificial Sequence Example of DNA encoding Sequence ID No. 41.
acccgcctattt

SEQ ID NO: 49 - DNA, 12 bp, Artificial Sequence
  Description of Artificial Sequence Example of DNA encoding Sequence ID No. 41.
acgcgtcttttt

SEQ ID NO: 50 - DNA, 12 bp, Artificial Sequence
  Description of Artificial Sequence Example of DNA encoding Sequence ID No. 41.
acacgtctattc

SEQ ID NO: 51 - protein, 21 aa, Artificial Sequence
  Description of Artificial Sequence This peptide bound to L2/HNK-1 carbohydrate in a concentration-dependent manner and inhibited HNK-1-mediated neural cell adhesion to lamin.
KGVSSRSYVGCIKNLEISRST

SEQ ID NO: 52 - protein, 21 aa, Unknown
  Description of Unknown Organism This protein sequence is available in public sequence database. It possesses homologous sequences with Seq ID No. 51.
KGVSSRSYVGCIKNLEISRST

SEQ ID NO: 53 - protein, 21 aa, Unknown
  Description of Unknown Organism This protein sequence is available in public sequence database. It possesses homologous sequences with Seq ID No. 51.
RGVTTKSFVGCIKNLEISRST

SEQ ID NO: 54 - protein, 21 aa, Unknown
  Description of Unknown Organism This protein sequence is available in public sequence database. It possesses homologous sequences with Seq ID No. 51.
PEVNLKKYSGCLKDIEISRTP

SEQ ID NO: 55 - protein, 20 aa, Unknown
  Description of Unknown Organism This protein sequence is available in public sequence database. It possesses homologous sequences with Seq ID No. 51.
KVEGVWTWVGTNKSLTEEAK

SEQ ID NO: 56 - protein, 21 aa, Unknown
  Description of Unknown Organism This protein sequence is available in public sequence database. It possesses homologous sequences with Seq ID No. 51.
RASSSKSWITFDLKNKEVSVK

SEQ ID NO: 57 - protein, 16 aa, Unknown
  Description of Unknown Organism This protein sequence is available in public sequence database. It possesses homologous sequences with Seq ID No. 51.
GTFKERIQWVGDPSWK

SEQ ID NO: 58 - protein, 22 aa, Artificial Sequence
  Description of Artificial Sequence This is the L2 Binding Protein Consenus Sequence.
XXXXXXXXXXXXXXXXXXXXXX

(19) United States
(12) Patent Application Publication
(10) Pub. No.: US 2003/0100508 A1
(43) Pub. Date: May 29, 2003

(54) Carbohydrate epitope mimic compounds and uses thereof

(76) Inventors: Maryline Simon, Baar (CH); Melitta Schachner, Hamburg (DE); Timothy J. Neuberger, Dobbs Ferry, NY (US); Uri Herzberg, Yorktown Heights, NY (US)

Correspondence Address: KLAUBER & JACKSON, 411 HACKENSACK AVENUE, HACKENSACK, NJ, 07601

(21) Appl. No.: 10/186,867

(22) Filed: Jul. 1, 2002

Related U.S. Application Data
(63) Continuation of application No. 09/511,956, filed on Feb. 23, 2000, now abandoned.
(60) Provisional application No. 60/121,327, filed on Feb. 24, 1999.
(60) Provisional application No. 60/155,492, filed on Sep. 23, 1999.

(51) Int. Cl. A61K 38/10; C07K 7/08

(52) U.S. Cl. 514/14; 530/326

(57) ABSTRACT

This invention provides carbohydrate epitope mimic compounds, particularly peptides, and analogs and variants thereof. In particular, the compounds and peptides of the present invention mimic the carbohydrate epitope GlcAβ1→3Galβ1→4GlcNAc or sulfate -3GlcAβ1→3Galβ1→4GlcNAc, or the L2/HNK1 carbohydrate epitope. This invention provides an isolated peptide comprising an amino acid sequence of a carbohydrate epitope mimic peptide in which the amino acid sequence is set forth in any of SEQ ID NOS: 1-8, 27-38, 39, 40 and 41, including variants, analogs and active fragments thereof. The invention further provides an isolated nucleic acid encoding a peptide comprising an amino acid sequence of a carbohydrate epitope mimic peptide. This invention provides pharmaceutical compositions and diagnostic and therapeutic methods of use of the isolated polypeptides and nucleic acids, particularly in modulating or mediating cell-cell adhesion and viral infection and the processes and events mediated thereby. Assays for compounds which mimic, alter or inactivate the polypeptides of the present invention for use in therapy are also provided.

### CROSS REFERENCE TO RELATED APPLICATIONS

The present application is a continuation-in-part of copending application Serial No. 60/121,327 filed Feb. 24, 1999, of copending application Serial No. 60/155,492 filed Sep. 23, 1999, of which the instant application claims the benefit of the filing date pursuant to 35 U.S.C. §119, and which is incorporated herein by reference in its entirety.

### FIELD OF THE INVENTION

The present invention relates generally to carbohydrate epitope mimic compounds, particularly peptides, to variants, analogs and active fragments thereof and to nucleic acids encoding such peptides, variants, analogs and active fragments. In particular, the peptides of the invention mimic the carbohydrate epitope GlcAβ1→3Galβ1→4GlcNAc or sulfate -3GlcAβ1→3Galβ1→4GlcNAc, or the L2/HNK1 carbohydrate epitope. The invention also relates to diagnostic, therapeutic and pharmaceutical compositions and uses of such compounds, particularly peptides, variants, analogs and active fragments thereof, and nucleic acids encoding such peptides, variants, analogs and active fragments, in modulating or mediating cell-cell adhesion and the processes and events mediated thereby.

### BACKGROUND OF THE INVENTION

The L2/HNK-1 Carbohydrate Epitope

Antibodies Recognizing L2/HNK-1

In 1981, Abo and Balch isolated a monoclonal IgM antibody directed against a membrane antigen from a cultured human T cell line (Abo and Balch, (1981) J. Immunology 127:1024-1029). This antibody was shown to react with 10% of blood lymphocytes and to recognize an antigen specific to human natural killer (NK) and killer (K) cells, thus the name HNK-1. NK and K cells are specialized lymphocytes that serve important roles in the surveillance of tumors and virus-infected cells. In the same study it was mentioned that the HNK-1 epitope was resistant to proteolysis, suggesting that the epitope was of non-proteinaceous nature. It was later shown that the antigen is a carbohydrate (Kruse et al, 1984).

The HNK-1 epitope is expressed predominantly on glycolipids and glycoproteins from nervous tissue (McGarry et al., (1983) Nature 306:376-378; Ilyas et al., (1984) Biochem. Biophys. Res. Comm. 122:1206-1121, Kruse et al., (1984) Nature 311.: 153-155; Yuen et al., (1997) J. Biol. Chem. 272:8924-8931). The expression pattern of the HNK-1 carbohydrate in both the central and peripheral nervous system is spatially and developmentally regulated (Wemecke et al., (1985) J. Neuroimmunol. 9:115-130; Holley and Yu -(1987) Dev. Neurosci. 9:105-19; Prasadarao et al., (1990) J. Neurochem. 55:2024-2030; Chou et al., (1991) J. Neurochem. 57:852-859; Löw et al., (1994) Eur. J. Neurosci. 6:1773-1781; Jungalwala (1994) Neurochem. Res. 19:945-957). The HNK-1 carbohydrate epitope is carried by many, but not all, neural recognition glycoproteins, and is involved in homo- and heterophilic binding of these proteins (for a review, see Schachner and Martini (1995) Trends Neurosci. 18:183-191). Of particular note is the association of the epitope with Schwann cells myelinating motor but not sensory axons (Löw et al., (1994) Eur. J. Neurosci. 6:1773-1781), where it may be involved in the preferential reinnervation of muscle nerves by motor axons after lesion (Martini et al., (1992) Eur. J. Neurosci. 4:628-639; Martini et al., (1994) J. Neurosci. 14:7180-7191).

In addition to the mouse HNK-1 antibody, there are several other antibodies recognized the L2/HNK-1 carbohydrate. Rat monoclonal antibodies isolated after immunization with a fraction enriched in plasma membrane include 334 (IgM), 336 (IgG), 349 (IgM), 344 (IgM), and 392 (IgM). The antibody L2-412 (IgG) was obtained by immunization with a membrane-derived glycoprotein fraction from mouse brain (Kruse et al, 1984, Noronha et al, 1986, Schachner et al, 1989). These antibodies react with glycoproteins and glycolipids carrying the L2/HNK-1 carbohydrate; thus, it is likely that they recognize the same or a closely related carbohydrate structure (Noronha et al, 1986). However, there are small differences in the staining intensity produced by the various monoconal antibodies, probably reflecting differences in affinities or small qualitiative differences among the epitopes recognized by the antibodies (Noronha et al, 1986).

Another group of monoclonal antibodies recognizing the L2/HNK- carbohydrate is the human IgM detected in the serum of some patients with neuropathies. The IgM was shown to bind to human myelin-associated glycoprotein (MAG); and the antigenic determinant reacting with the IgM was in the carbohydrate part of the MAG molecules (Ilyas et al, 1984, Quarles et al, 1992). The fine specificities of these human antibodies have been investigated; striking differences were seen in the structural requirements for binding: Some IgMs needed the sulfate group while others did not (Ilyas et al, 1990). It has been suggested that the epitope recognized by these IgM antibodies may be an important target in paraproteinemic neuropathies. The capacity of the human anti-MAG antibodies to cause demyelination under appropriate conditions has been demonstrated in chicken. Transfusion of chickens with monoclonal IgM antibodies isolated from human patients causes peripheral demyelination characteristic of the human syndrome, confirming the involvement of the antibodies in damaging nervous tissues (Tatum et al, 1993).

Structure of the L2/HNK-1 Carbohydrate

The L2/HNK-1 carbohydrate is found in glycolipids, glycoproteins, and proteoglycans. The structure which reacts with HNK-1 antibody was first described by Chou and Jungalwala for the major antigenic glycolipid present in human peripheral nerve. The compostion, sugar linkage, configuration and position of the sulfate group, were characterised as sulfate -3GlcAβ1-3) Galβ(1-4) GlcNAcβ(1-3) GalNAcβ(1-3) Galβ(1-4) Glcβ(1-1)-ceramide for SGGL-1 and as sulfate -3GlcAβ(1-3) Galβ(1-4) GlcNAcβ(1-3) Galβ(1-4) GlcNAcβ(1-3) Galβ(1-4) Glcβ(1-1)-ceramide for SGGL-2. (Chou et al, 1986).

More recently, the structure of a L2-412-reactive carbohydrate epitope of bovine peripheral myelin glycoprotein (PO) has been elucidated (Voshol et al, 1996). It contains the same terminal trisaccharide as in the glycolipid structure determined by Chou and Jungalwala, suggesting that this structure is sufficient for its immunoreactivity and may be a key element in the structure.

The enzymes involved in the biosynthesis of the L2/HNK-1 carbohydrate have been studied at the biochemical level. Glycosyltransferase (Chou et al, 1996), galactosyltransferase (Chou et al, 1994), glucuronyltransferase (Chou et al, 1991) and sulfotransferase (Chou et al, 1996) have been studied using crude enzyme preparations. Two of them have been purified, i.e., an N-acetylglucosaminyltransferase (Chou et al, 1993), and a glucuronyltransferase respectively (Oka et al, 1992). Recently a cDNA encoding the glucuronyltranserase involved in the biosynthesis of the L2/HNK-1 carrying glycoprotein has been cloned (Terayma et al, 1997). A cDNA coding for a sulfotransferase responsible for coupling the sulfate group to the C-3 position of the GlcA residue was first cloned in our laboratory (Bakker et al, 1997) and shortly thereafter by another group (Ong et al, 1998). Both cDNAs probably encode species homologs (rat and human), since 90% of their amino acid residues are identical.

Determination of the structure of the glycolipid (Chou et al., (1986) J. Biol. Chem. 261:11717-11725) and glycoprotein (Voshol et al., (1996) J. Biol. Chem. 271:22957-22960) forms has shown that both carry sulfate -3-GlcAβ1→3Galβ1→4GlcNAc at the nonreducing end. The minimal requirement for recognition by HNK-1 is unknown, but the antibody only binds to the sulfated form (Ilyas et al., (1990) J. Neurochem. 55:594-601). Several other monoclonal antibodies have been isolated that recognize identical or similar structures (Kruse et al., (1984) Nature 311:153-155; Noronha et al., (1986) Brain Res. 385:237-244); of these, L2-412 is important for this study, because it also recognizes the non-sulfated form of the carbohydrate (Schmitz et al., (1994) Glycoconjugate J. 11:345-352).

The mouse HNK-1 and the L2-412 antibodies have been studies using synthetic glcolipids with regard to their requirement for binding. The HNK-1 antibody shows an absolute requirement for the sulfate group (Ilas et al, 1990). In contrast, the L2-412 antibody recognizes both the sulfated and the non-sulfated form of the carbohydrate structure (Schmitz et al, 1994).

Appearance of the L2/HNK-1 Carbohydrate

The L2/HNK-1 carbohydrate is found on a large number of molecules both in the CNS and PNS. It has been hypothesized that molecules expressing this epitope could be involved in adhesion, although it has not yet been proven in the case of Drosophila melanogaster, zebrafish and lymphocytes. Table 1 summarizes, in a non-exhaustive list, the diversity of molecules carrying the L2/HNK-1 carbohydrate. The presence of this carbohydrate in groups as diverse as mammals, fish, and insects, may indicate the importance of this carbohydrate.

TABLE 1
Presence of the L2/HNK-1 carbohydrate
on various adhesion molecules and in various species

| Mammals | Reference |
|---|---|
| MAG | (McGarry et al, 1983) |
| L1, NCAM | (Kruse et al, 1984) |
| PO | (Griffith et al, 1992) |
| MOG | (Burger et al, 1993) |
| OMgp | (Mikol et al, 1990) |
| PMP22 | (Suter et al, 1995; Snipes et al, 1993) |
| Tenascin R + C | (Kruse et al, 1985) |
| SAG | (Dieperink et al, 1992) |
| PI-GP150 | (Yoshihara et al, 1991; Yoshihara et al, |
| (Telencephaline) | 1994) |
| Glycolipids (SGGLs) | (Chou et al, 1985; Nair et al, 1997: Nair et |
| | al, 1993) |
| Proteoglycans | (Kruegger et al, 1992) |
| Integrins | (Pesheva et al, 1987) |
| Other species | |
| Zebrafish | (Metcalfe et al, 1990) |
| Electric ray | (Vogel et al, 1991) |
| Calliphora vicina, | (Dennis et al, 1988); Dennis et al, 1991) |
| Drosophila | |
| melanogaster | |

The carbohydrate appears therefore on a variety of molecules. It is unclear whether the carbohydrate has the same function on different molecules or whether its function depends on the molecule carrying it at various regions and stages of development of the nervous system.

Although many proteins may carry the L2/HNK-1 epitope, it is difficult to determine at which developmental stage and in which brain region on a particular protein carries this epitope. In the case of N-CAM, for example, only a subpopulation of the molecules carries the L2/HNK-1 epitope (Kruse et al, 1984). This is also the case for L1 (Faissner et al, 1987), MAG (Poltorak et al, 1987) and PO (Burger et al, 1990). The expression of the glycoproteins carrying the L2/HNK-1 carbohydrate has been studied in the rat (Chou et al, 1991). In the cerebellum and in the cerebral cortex, the glycoproteins carrying the L2/HNK-1 carbohydrate are found at embryonic day 19 (ED 19) and continue to be expressed in the adult. Yoshihara et al (Yoshihara et al, 1991; Yoshihara at al, 1994) have shown with their studies on the glycoprotein PI-GP 150 that the L2/HNK-1 carbohydrate moiety can be regulated independently of the expression of the protein backbone, and that its expression shows segmental differences. Thus, using monoclonal HNK-1 antibodies, they have shown that the telencephalon expresses the L2/HNK-1 epitope constitutively; in the midbrain, by contrast, its expression decreases after postnatal day 7 (PD7) and becomes completely absent in the adult myencephalon and metencephalon.

As mentioned earlier, the L2/HNK-1 epitope is present not only in glycoproteins, but also in proteoglycans and glycolipids, which makes it difficult to determine the spatial expression of the L2/HNK-1 epitope in a specific molecule. At a gross level, it was shown that in embryonic rat and mouse brain immunoreactivity with L2/HNK-1 antibodies has a similar distribution but appears at slightly different embryonic ages. Thus, the expression of the major HNK-1-reactive glycolipids studied in rat by Schwarting (Schwarting et al, 1987) is in good correlation with the more precisely located developmental expression of the HNK-1-reactive glycolipids in the rat (Chou et al, 1991). In the cerebral cortex, the L2/HNK-1-carrying glcolipids SGGLs-1 and -2, are expressed maximally around ED19, decline by PD5, and almost completely disappear by PD20. In the cerebellum, the developmental pattern of the L2/HNK1-carrying glycolipids showed two phases, with the first maxium near birth, a decrease until PD7, and then a second maximum of expression starting at PD 10 and peaking at PD20 and remaining constant in the adult. The studies on L2/HNK-1-carrying molecules in the mammalian nervous system may be summarized as follows: The two sulfoglucuronyl glycolipids (SGGLs) are expressed in the cerebral cortex during neonatal development, but disappear in the adult. However, in the PNS and cerebellum, they are also found in the adult. By contrast, L2/HNK-1-reactive glycoproteins continue to be expressed throughout the nervous system in adulthood (Chou et al, 1991). It should however, be noted that the rat femoral nerve was shown to be HNK-1 negative using the HNK-1 antibodies. Reactivity to other L2/HNK-1 recognizing antibodies has not been studied.

Roles of the L2/HNK-1 Carbohydrate

Embryonic Development

The integrins are a family of glycoproteins that can carry the L2/HNK-1 carbohydrate.

They interact with a wide variety of ligands, including extracellular carbohydrate. They interact with a wide variety of ligands, including extracellular matrix glycoproteins such as laminin. They participate in cell-matrix and cell-cell adhesion in important processes such as embryonic development (Hynes et al, 1987). In this capacity, integrins are presumed to function in cell migration in embryos. During their migration, neural crest cells encounter various tissues and extracellular matrix molecules surrounding these tissues. The HNK-1 antibody recognizes a carbohydrate epitope on the surface of migrating neural crest cells which is closely related if not identical to the L2/HNK-1 carbohydrate. The role of the carbohydrate in chicken was investigated in vivo and in vitro by treatment with HNK-1 antibody. Addition of the HNK-1 antibody to neural tube explants in tissue culture, caused neural crest cells to detach from laminin substrate and alter their morphology. Injection of the antibody into embryos caused abnormalities in neural cell migration and development. The timing appeared to be critical, indicating that the HNK-1 antibody selectively perturbs the early stages of neural crest migration (Bronner-Fraser et al, 1987). A possible role for the L2/HNK-1 epitope in development of the enteric nervous system in rats was found by Newgreen and co-workers (Newgreen el al, 1995). As development proceeds, the gut is colonized by neural crest cells which will form the enteric nervous system. The authors used the HNK-1 antibody as a marker for this type of cells in rat. The enteric neurons appearing during this developing period were also recognized by the HNK-1 antibody suggesting the possible involvement of the carbohydrate epitope in rat embryonic development.

Cellular Interactions and Adhesion

The L2/HNK-1 carbohydrate was shown to be involved in cell-cell adhesion (Keilhauer et al, 1985). A homogeneous population of neurons and a homogeneous population of astrocytes were isolated from early postnatal mouse cerebellum and tested for adhesion in the presence or absence of L2-412 antibodies (and other antibodies). It was suggested that the L2/HNK-1 carbohydrate can act as a ligand in cell adhesion (Kunemund et al, 1988), and that it is more important for cell-substrate than for cell-cell interactions. More recently, Hall and co-workers (Hall et al, 1993) showed that L2/HNK-1 carbohydrate and heparin were using different binding sites on laminin and were thus implicated in different aspects of neural cell adhesion to laminin. In another experiment, analysis of crude membrane fractions of small cerebellar neurons with L2-412 antibody demonstrated that these neurons express one major L2-412-immunoreactive glycoprotein, which was identified as neural cell adhesion molecule L1. The binding of L1 to laminin could be reduced in the presence of Fab fragments of the L2-412 antibody, showing that L1 binds directly to laminin via the L2/HNK-1 carbohydrate. The authors could show in competition and inhibition assays that glycolipids carrying the carbohydrate were also involved in cell adhesion to laminin (Hall et al, 19.95, Hall et al, 1997 a; Hall et al, 1997 b).

Another example of cellular interactions involving the L2/HNK-1 carbohydrate is seen with the binding of the HNK-1-reactive glycolipids to selectins. Selectins (E, L, and P) are a family of structurally and functionally related cell surface adhesion proteins that bind carbohydrates. They are implicated in adhesive interactions with cells of the vascular endothelium. In an experiment designed to investigate which carbohydrate ligand is responsible for selectin-mediated cell adhesion, it was shown that the glycolipids carrying the L2/HNK-1 carbohydrate are ligands for L-selectin and for P-selectin, but not E-selectin, even though all three selectins share considerable structural similarity. Another interesting point raised in this study is that removal of the sulfate group from the glycolipid did not significantly decrease its binding to either P- or L-selectin, demonstrating that the sugar core of the L2/HNK-1 carbohydrate epitope is sufficient for P-selectin and L-selectin binding (Needham et al, 1993).

Formation and Maintenance of Blood-Brain Barrier

The endothelial cells of brain microvascular origin (BMECs) are believed to form the structural basis of the blood-brain barrier. They are the only cells in the nervous system that are continuously exposed to blood. In an interesting experiment (Kanda et al, 1995), it was demonstrated that the treatment of BMECs with an inflammatory cytokine could induce the accumulation of glycolipids carrying the L2/HNK-1 carbohydrate. A significant larger number of human lymphocytes attached to the stimulated BMECs compared to the non-stimulated BMECs, and this adherence was effectively blocked by pre-incubation of 1) the lymphocytes with an anti-L-selectin antibody, or 2) the BMECs with a monoclonal antibody against SGGLs. These results suggest that glycolipids carrying the L2/HNK-1 carbohydrate act as one of the ligands for 1-selectin in inflammatory disorders of CNS/PNS, and that they regulate the attachment of activated lymphocytes and their subsequent invasion of the CNS and PNS. The authors suggested that, since a number of glycoconjugates possessing the L2/HNK-1 epitope have been implicated in cellular adhesion (see section 1.5.2.), the SGGLs, through the L2/HNK-1 carbohydrate, may be involved in intercellular adhesion of BMECs for the formation of the blood-brain-barrier and may play a critical role in maintenance of the barrier function (Kanda et al, 1995).

Homophilic Interaction

Peripheral myelin glycoprotein (PO) is an example of an adhesion molecule that engages in homophilic binding (that is, it binds to itself). The L2/HNK-1 carbohydrate expressed on a subset of PO molecules has been shown to be involved in this binding: Binding could be partially inhibited by antibodies to the L2/HNK-1 epitope and by L2/HNK-1 carbohydrate (but not other carobohydrates). Inhibition was also seen with polyclonal antibodies reacting with the protein backbone of PO, indicating that both protein and carbohydrate structures are involved in the binding of PO to PO, and that PO acts both as presenter and a receptor of the L2/HNK-1 carbohydrate (Griffith et al, 1992).

Using a cell line expressing unglycosylated PO, evidence was provided that PO must be glycosylated to be adhesive, and also that glycosylation of both PO molecules is necessary for homophilic adhesion to take place. In the same study it was suggested that carbohydrates play a role in positioning PO relative to the membrane (Filbin et al, 1993), indicating that another function of the oligosaccharide moiety of PO may be to stabilize the orientation of the protein (Quarles et al, 1997).

Outgrowth of Motor Axons and L2/HNK-1 Carbohydrate in Regeneration in the PNS

Preferential motor reinnervation has been studied mainly in the femoral nerve. The term describes the ability of motor axons regenerating in a mixed nerve such as the femoral nerve to selectively reinnervate a motor branch. This occurs even if the two branches of the nerve are intentionally misaligned, suggesting that specific interactions, independent of mechanical influences, occur between regenerating motor axons and the distal branch (Brushart et al, 1990).

In the mouse femoral nerve, the L2/HNK-1 carbohydrate is selectively expressed on the Schwann cells and Schwann cell basement membrane of the motor branch, but is rarely found in the sensory branch (Martini et al, 1988). It persists in these locations during and after Wallerian degeneration (Martini et al, 1992). Analysis of the myelin of the muscle and cutaneous branches of the adult mouse femoral nerves by immunochemical methods showed that the L2/HNK-1 carbohydrate was detectable on both SGGL-1 and SGGL-2. Furthermore, the glycoprotein uniquely L2-412-immunoreactive in the muscle nerve was identified as MAG (Low et al, 1994). The L2/HNK-1 carbohydrate also selectively promotes outgrowth of neurites from motor axons in vitro. This was demonstrated using cryostat sections of femoral nerve sensory and motor branches on which motor neurons were allowed to grow. Neurites preferentially elongate on the motor branch expressing the L2/HNK-1 as compared to the sensory branch scarcely expressing L2/HNK-1. In contrast, neurites extending from sensory neurons reached the same length on both substrates (Martini et al, 1992). In the mouse, the L2/HNK-1 carbohydrate thus selectively marks the motor pathway. It was shown however that the motor branch of the rat femoral nerve was not HNK-1 positive (Levi et al, 1994: Schuller-Petrovic et al, 1983), which might be explained by the fine specificity of the particular antibodies used (Yamawaki et al, 1996). In the mouse, it is present in the proper cellular location and at the proper time to influence regeneration, and has a selective effect on motor neurons in vitro. Furthermore, the L2/HNK-1 carbohydrate remains strongly expressed for at least 14 days in the denervated distal nerve stump of the motor branch, whereas the sensory branch remains negative (Martini et al, 1992).

The femoral nerve receives sensory axons from dorsal root ganglia (DRG) and motor axons from the ventral root. Distally, it divides into a cutaneous branch (sensory axons only) and a muscle branch (both sensory and motor axons). In another series of experiments, the femoral nerve was deefferented by transection of the ventral root or deafferented by removal of DRG. In the deafferented muscle branch, the pattern of L2-412- immunoreactivity of Schwann cells was similar to that found in the non-deafferented control. In contrast, in deafferented mice, L2-412-immunoreactivity was markedly decreased and only a few Schwann cells were positive, indicating the importance of motor axons for L2-412-immunoreactivity on myelinating Schwann cells. In the next experiment, the femoral nerve was proximally transected and regenerating motor axons were prevented from reaching muscles to avoid target influence that could explain the strong expression of L2/HNK-1 by the motor branch in contrast to the poor expression of the epitope in the cutaneous branch. However, L2/HNK-1 expression remained prominent in the target-deprived muscle branch, while the target-deprived cutaneous branch still showed little L2-412-immunoreactivity suggesting that expression of L2/HNK-1 by Schwann cells of the muscle branch is independent of target innervation and must depend on axon-Schwann cell interaction in the nerve. To complete these observations, experiments introducing grafts of different types were done. In these experiments, cutaneous and muscle branches of the femoral nerve were removed from one leg and inserted into the contralateral femoral nerve as grafts. In one group (graft group), branches were grafted with the muscle and the cutaneous nerve graft inserted in the corresponding muscle or cutaneous branch, respectively. In a second group (reversed group), the grafts were inserted with the cutaneous nerve grafts in the muscle branch and the muscle nerve grafts into the cutaneous branch. A particular interesting pattern was observed when a cutaneous graft was introduced in the muscle branch: in the cutaneous nerve graft itself, L2/HNK-1 was poorly expressed but the muscle branch distal to the graft strongly expressed the L2/HNK-1, although the distal muscle branch was reinnervated by the same axons that had penetrated the cutaneous graft. In the graft group, in which the muscle graft was introduced into the corresonding muscle branch, highly L2-412-immunoreactive myelinating Schwann cells were found indicating, that the grafting per se did not interfere with the capacity of Schwann cells to express L2/HNK-1. These combined observations indicate that Schwann cells previously associated with motor axons retain some of their acquired properties and express the L2/HNK-1carbohydrate epitope more effectively than Schwann cells that have previously myelinated sensory axons, appearing to “remember” their previous axonal association, Schwann cell-mediated L2/HNK-1 expression may thus influence preferential reinnervation of muscle nerve by regenerating motor axons of the peripheral nervous sytem (Martini et al, 1994).

Neural Cell Adhesion Molecules

The ability of neurons to extend neurites is of prime importance in establishing neuronal connections during development. It is also required during regeneration to re-establish connections destroyed as a result of a lesion. Neurites elongate profusely during development both in the central and peripheral nervous systems of all animal species (Cajal (1928) Degeneration and regeneration in nervous system, Oxford University Press, London). This phenomenon pertains to axons and dendrites. However, in adults, axonal and dendritic regrowth in the central nervous system is increasingly lost with evolutionary progression.

In the peripheral nervous system, after infliction of a lesion, axons of all vertebrate species are able to regrow (Cajal (1928); Martini (1994) J. Neurocytol. 23:1-28). However, in mammals, neurite regrowth following damage is limited to neuritic sprouting. Regrowth of neuronal processes is, however, possible in lower vertebrate species (Stuermer et al. (1992) J Neurobiol. 23:537-550). In contrast, in the central nervous system, most, if not all neurons of both higher and lower vertebrate adults possess the potential for neurite regrowth (Aguayo (1985) “Axonal regeneration from injured neurons in the adult mammalian central nervous system,” In: Synaptic Plasticity (Cotman, C. W., ed.) New York, The Guilford Press, pp. 457-484.)

Glial cells are the decisive determinants for controlling axon regrowth. Mammalian glial cells are generally permissive for neurite outgrowth in the central nervous system during development,(Silver et al. (1982) J. Comp. Neurol. 210:10-29; Miller et al. (1985) Develop. Biol. 111:35-41; Pollerberg et al. (1985) J. Cell. Biol. 101: 1921-1929) and in the adult peripheral nervous system (Fawcett et al. (1990) Annu. Rev. Neurosci 13:43-60). Thus, upon infliction of a lesion, glial cells of the adult mammalian peripheral nervous system can revert to some extent to their earlier neurite outgrowth-promoting potential, allowing them to foster regeneration (Kalderon (1988) J. Neurosci Res. 21:501-512, Kliot et al. “Induced regeneration of dorsal root fibres into the adult mammalian spinal cord,” In: Current Issues in Neural Regeneration, New York, pp. 311-328; Carlstedt et al. (1989) Brain Res. Bull. 22:93-102). Glial cells of the central nervous system of some lower vertebrates remain permissive for neurite regrowth in adulthood (Stuermer et al. (1992) J. Neurobiol. 23:537-550). In contrast, glial cells of the central nervous system of adult mammals are not conducive to neurite regrowth following lesions.

Several recognition molecules which act as molecular cues underlying promotion and/or inhibition of neurite growth have been identified (Martini (1996). Among the neurite outgrowth promoting recognition molecules, are neural cell adhesion molecules belonging to the immunoglobulin superfamily, and particularly to those members that mediate Ca²⁺-independent neuronal cell adhesion, of which L1, N-CAM and myelin-associated glycoprotein are particular members. Other cell adhesion molecules which may also influence CNS neural growth include laminin, fibronectin, N-cadherin, BSP-2/D-2 (mouse N-CAM), 224-1A6-A1, L1-CAM, NILE (rat L1), Nr-CAM, TAG-1 (axonin-1), Ng-CAM and F3/F11 /contactin. The prominent role played in mediating neurite outgrowth by the neural adhesion molecule L1 has been demonstrated (Schachner (1990) Seminars in the Neurosciences 2:497-507). L1-dependent neurite outgrowth is mediated by homophilic interaction. L1 enhances neurite outgrowth on L1 expressing neurites and Schwann cells, and L1 transfected fibroblasts (Bixby et al. (1982) Proc. Natl. Acad. Sci. USA. 84:2555-2559; Chang et al. (1987) J. Cell. Biol. 104:355-362; Lagenaur et al. (1987) Proc. Natl. Acad. Sci. USA 84:7753-7757; Seilheimer et al. (1988) J. Cell. Biol. 107:341-351; Kadmon et al. (1990a) J. Cell. Biol. 110:193-208; Williams et al. (1992) J. Cell. Biol. 119:883-892). Expression of L1 is enhanced dramatically after cutting or crushing peripheral nerves of adult mice (Nieke et al. (1985) Differentiation 30:141-151; Martini et al. (1994a) Glia 10:70-74). Within two days L1 accumulates at sites of contact between neurons and Schwann cells being concentrated mainly at the cell surface of Schwann cells but not neurons (Martini et al. (1994a)). Furthermore, the homophilic binding ability of L1 is enhanced by molecular association with the neural cell adhesion molecule N-CAM, allowing binding to occur through homophilic assistance (Kadmon et al. (1990a); Kadmon et al. (1990b) J. Cell Biol. 110:209-218 and 110: 193-208; Horstkorte et al. (1993) J. Cell. Biol. 121:1409-1421). Besides its neurite outgrowth promoting properties, L1 also participates in cell adhesion (Rathjen et al. (1984) EMBO J. 3: 1-10; Kadmon et al. (1990b) J. Cell. Biol. 110:209-218; Appel et al. (1993) J. Neurosci., 13:4764-4775), granule cell migration (Lindner et al. (1983) Nature 305:427-430) and myelination of axons (Wood et al. (1990) J. Neurosci 10:3635-3645).

L1 consists of six immunoglobulin-like domains and five fibronectin type III homologous repeats. L1 acts as a signal transducer, with the recognition process being a first step in a complex series of events leading to changes in steady state levels of intracellular messengers. The latter include inositol phosphates, Ca²⁺, pH and cyclic nucleotides (Schuch et al. (1990) Neuron 3:13-20; von Bohlen und Halbach et al. (1992) Eur. J. Neurosci. 4:896-909; Doherty et al. (1992) Curr. Opin. Neurobiol. 2:595-601) as well as changes in the activities of protein kinases such as protein kinase C and pp60^{c-arc} (Schuch et al. (1990) Neuron 3:13-20; Atashi et al. (1992) Neuron 8:831-842). L1 is also associated with a casein type II kinase and another unidentified kinase which phosphorylates L1 (Sadoul et al. (1989) J Neurochem 328:251-254). L1 -mediated neurite outgrowth is sensitive to the blockage of L type Ca²⁺ channels and to pertussis toxin. These findings indicate the importance of both Ca²⁺ and G proteins in L1 -mediated neurite outgrowth (Williams et al. (1992) J. Cell. Biol. 119:883-892). L1 is also present on proliferating, immature astrocytes in culture and neurite outgrowth is promoted on these cells far better than on differentiated, L1immunonegative astrocytes (Saad et al. (1991) J. Cell. Biol. 115:473-484). In vivo, however, astrocytes have been found to express L1 at any of the developmental stages examined from embryonic day 13 until adulthood (Bartsch et al. (1989) J. Comp. Neurol 284:451-462; and unpublished data).

Natural Killer Cells and the Immune System

As noted above, the HNK-1 antibody was so named by its characteristic ability to recognize an antigen specific to human natural killer (NK) and killer (K) cells. NK and K cells are specialized lymphocytes that have been implicated in viral immunity and in defense against tumors. NK cells have also been shown to play a role in the graft-versus-host reaction and these cells may contribute to some of the skin lesions and intestinal wall damage observed. The cells make up approximately 10% of the recirculating lymphocyte population.

NK cells are involved in the early response to infection with certain viruses and intracellular bacteria. NK activity is stimulated by IFN-alpha, IFN-beta and IL-12. In the course of a viral infection, these cytokines rapidly rise, followed closely by a wave of NK cells that peaks in about 3 days. NK cells provide the first line of defense to virus infection, controlling viral replication during the time required for activation, proliferation and differentialtion of cytotoxic T cells (CTLs) at about day 7. For a review of NK cells and CTLs, see Berke, G. 1995 (Berke G. Immunol. Today 16, 343 (1995)). The importance of NK cells in defense against viral infections is illustrated by the case report of a young woman who completely lacked NK cells. Despite normal T and B cell counts, this woman suffered severe varicella virus infections and life-threatening cytomegalovirus infection.

There are a number of recognized viruses that infect or affect the immune system, particularly lymphocytes, including human immunodeficiency virus (HIV) and human T-cell lymphocyte virus (HTLV). HIV can also infect the nervous system and is associated with AIDS-dementia. There are also recognized viruses which can be neurologically associated and can cross or disrupt the blood brain barrier, leading in some cases to viral encephalitis, neural cell death, paralysis or dementia.

Natural killer cells appear to kill tumor cells and virus infected cells by a process similar to that employed by CTLs. The cytoplasm of NK cells contains numerous granules containing perforin and granzymes. After an NK cells adheres to a target cell, degranulation occurs with release of perforin and granzymes at the junction of the interacting cells. NK cells have also been shown to mediate target-cell destruction by apoptosis. Importantly, and distinct from CTLs, NK cells do not express antigen-specific T cell receptors or CD3 and target-cell recognition by NK cells is not MHC restricted.

NK cells can bind to antitumor antibodies bound to the surface of tumor cells and subsequently destroy the tumor, a process denoted antibody-dependent cell-mediated cytotoxicity (ADCC). NK cells have been shown to secrete tumor necrosis factor (TNF). In humans, Chediak-Higashi syndrome, an autosomal recessive disorder, is associated with an absence of NK cells and an increased incidence of lymphomas. Mice with an autosomal mutation called beige lack NK cells and are more susceptible than normal mice to tumor growth following injection with live tumor cells.

The HNK-1 antibody has been shown to detect antigens which are heavily expressed by benign prostatic hyperplasia and carcinoma of the prostate (Lipford, G. B. and Wright, G. L. Jr. Cancer Res. 51(9), 2296-3001 (1991)). This antibody also recognizes a number of human neuroblastoma lines and expression of the HNK-1 antigen on these lines can be slightly increased by retinoic acid-induced differentiation of the cells (McGarry, R. C. et al., Cancer Immunol Immunother 27(1), 47-52 (1988)).

Phage Display

Screening phage-displayed random peptide libraries offers a rich source of molecular diversity and represents a powerful means of identifying peptide ligands that bind a receptor molecule of interest (Cwirla et al, 1990; Devlin et al, 1990, Cortese et al, 1995). Phage expressing binding peptides are selected by affinity purification with the target of interest. This sytem allows a large number of phage to be screened at one time. Since each infectious phage encodes the random sequence expressed on its surface, a particular phage, when recovered from an affinity matrix, can be amplified by another round of infection. Thus, selector molecules immobilized on a solid support can be used to select peptides that bind to them. This procedure reveals a number of peptides that bind to the selector and that often display a common consensus amino acid sequence. Biological amplification of selected library members and sequencing allows the determination of the primary structure of the peptide(s).

Peptides are expressed on the tip of the filamentous phage M13, as a fusion protein with the phage surface protein pilus (at the N-terminus). Typically, a filamentous phage carries on its surface 3 to 5 copies of pili and therefore of the peptide. In such a system, no structural constraints are imposed on the N-terminus; the peptide is therefore free to adopt many different conformations, allowing for a large diversity. However, biases in the distribution of peptides in the library may be caused by biological selection against certain of the peptides, which could reduce the diversity of peptides contained in the library. In practice, this does not appear to be a significant problem. When randomly selected peptides expressed at the N-terminus of pli were analyzed (Cwirla et al, 1990), most amino acids appeared at each position of the variable peptide, indicating that no severe discrimination against particular amino acids had occurred. Selection against particular combinations of amino acids would however not have been detected in this analysis.

Peptide ligands identified by phage display screening frequently interact with natural binding site(s) on the target molecule, and often resemble the target's natural ligand(s). Although this system has been most often used to identify peptide epitopes recognized by antibodies, it has also been successfully used to find peptide mimics of carbohydrate molecules. Work directed towards using peptide mimics in place of carbohydrate antigens has been reviewed by Kieber-Emmons and colleagues (Kieber-Emmons et al, 1998). The demonstrated ability of a peptide to mimic a carbohydrate determinant indicates that, although mimicry is accomplished using amino acids in place of sugars, the specificity pattern can be reproduced.

Peptides that mimic glycosphingolipids have been found using a phage peptide library. Two monoclonal antibodies that recognize lactotetraosylceramide (Lc4Cer) and its isomer neolactotetraosylceramide (nLc4Cer) were used to find peptides that mimic the carbohydrate moieties of the two glycosphingolipids. It was also shown that the peptides are biologically active, in that they could modulate the activity of β-galactosidase (Take et al, 1997).

The pathogen Shigella flexneri is a bacterium responsible for the endemic form of shigellosis, a dysenteric syndrome characterized by bacterial invasion of the human colonic mucosa. The cell wall of this bacterium contains repeated saccharide units forming the O-antigen carbohydrate moiety of the capsular lipopolysaccharide. To overcome the weak antibody response typical of carbohydrate antigens, peptide mimics of the carbohydrate epitope were isolated using phage display technology. These mimics could act as immunogenic mimics, and were capable of inducing specific anti-carbohydrate antibodies (Phalipon et al, 1997).

Peptides that mimic HIV-associated carbohydrate forms have also been reported. Mouse antisera were generated against peptides that mimic a mucin-related carbohydrate epitope expressed on HIV. The authors showed that immunization with the peptide-mimics induces antibodies that cross-reacted with native HIV envelope proteins. The sera containing these antibodies could neutralise HIV-1 cell-free infection in vitro as well as the sera from patients infected with HIV-1 whereas normal human sera were ineffective in this viral neutralisation assay (Agadjanyan et al, 1997).

In another recent study, screening was carried out with the lectin 134 that binds to the sugar Galα (1,3) Gal antibodies to the Galα (1,3) Gal epitope. Human natural Galα (1,3) Gal antibodies and the lectin IB4 also reacted with peptides encoded by the human mucin gene MUC1 that can be up-regulated in breast cancer. Apostolopoulos and co-workers showed that immunization with the peptide-mimic DAHWESWL could induce anti-MUC1 responses and have an anti-tumor activity against MUC1 tumors in mice (Apostolopoulos et al, 1998).

Further such studies include: (a) a peptide mimic of a carbohydrate epitope of the Lewis Y antigen has been reported and contains the residues PWLY, which were shown to be critical for peptide binding to an antibody specific for the Lewis Y antigen (Hoess et al, 1993); (b) peptides that mimic the capsular polysaccharide of Neisseria meningitidis serogroup C generated an immune response that was able to protect mice against infection with a lethal dose of the encapsulated bacteria (Westerink et al, 1995); and (c) the carbohydrate binding site of the lectin concanavalin A was investigated and peptides that mimic the binding of methyl α-D-mannopyranoside to ConA were identified by screening a phage-displayed random hexa- or decapeptide library (Scott et al, 1992, Oldenburg et al, 1992). The peptides binding ConA were shown to contain the consensus sequence YPY (Oldenburg et al, 1992).

A major obstacle in the investigation of biological functions of complex carbohydrates is the availability of these compounds. They can often be isolated from biological sources in only minute amounts. For L2/HNK-1 carbohydrate, for example, the yield is approximately 2.5 mg per kg of beef cauda equina. Furthermore, material from cattle nerve would be unsuitable for any clinical application. The chemical synthesis of a complicated oligosaccharide structure, such as the L2/HNK-1 epitope, is a complicated and lengthy process (Nakano et al, 1991). The chemical synthesis requires a crucial coupling between a key glycoheptaosyl donor and a ceramide derivative, followed by the final introduction of a terminal sulfate group. The total synthesis requires 15 intermediate compounds and about 20 steps, of which several are very time consuming. A possible solution to this problem; is to mimic the carbohydrate by other compounds that are easier to prepare, e.g. peptides. The most promising way to find such peptides is by use of the random peptide phage display (RPPD) technology.

Therefore, in view of the aforementioned deficiencies attendant with prior art methods of making, synthesizing and characterizing carbohydrate epitopes and of activating or therapeutically using carbohydrate epitope recognizing molecules, including neural cell adhesion molecules, it should be apparent that there exists a need in the art for compounds or peptides capable of mimicking carbohydrate epitopes.

The citation of references herein shall not be construed as an admission that such is prior art to the present invention.

### SUMMARY OF THE INVENTION

In its broadest aspect, the present invention encompasses an isolated peptide which mimics the carbohydrate epitope GlcAβ1→3Galβ1→4GlcNAc or sulfate -3GlcAβ1→3Galβ1→4GlcNAc, and variants, analogs and active fragments thereof In a further aspect, the invention extends to compounds, particularly peptides, that are capable of mimicking the L2/HNK1 carbohydrate epitope. The compounds or peptides of the invention are further capable of interacting with or binding to molecules which interact with or bind to the L2/HNK1 carbohydrate epitope. Particular examples of such molecules are laminin, P-selectin, L-selectin, fibronectin, N-cadherin, myelin associated glycoprotein (MAG), neural cell adhesion molecules, N-CAM, BSP-2/D2 (mouse N-CAM), 224-1A6-A1, L1-CAM, NILE (rat L1), Nr-CAM, TAG-1 (axonin-1), Ng-CAM and F3/F11/contactin.

In a further embodiment, an isolated peptide is provided comprising an amino acid sequence X₁ X₂ X₃ X₄ X₅ L/V X₆ X₇ X₈ X₉ X₁₀ X₁₁ X₁₂ X₁₃ X₁₄, wherein each residue can be independently selected as follows (SEQ ID NO: 1):

X₁ is T, S, A or P;

X₂ is L, I, V, M, F, H, W or N;

X₃ is T, S, A, H, Y, F, W, N, D or E;

X₄ is R, Q, K, T, S or A;

X₅ is V, I, L, M, R, Q or K;

X₆ is T, S, A, Y, F, H, W, N, L, I, V or M;

X₇ is D, E, V, L, I, M, F, Y, H, W or N;

X₈ is V, I, L, M, S, A, T, R, Q or K;

X₉ is Y, F, H, W, D, E, I, V, L, M or N;

X₁₀ is R, Q, K, W, Y, F, H, N, V, I, L, M or G;

X₁₁ is G, Y, F, H, W, N, S, A, T, I, V, L, M;

X₁₂ is R, Q, K, H, N, Y, F, W, I, V, L or M;

X₁₃ is L, V, I, M, T, S or A; and

X₁₄ is S, T, A, P, G, R, Q or K;

and variants, analogs and active fragments thereof

In a still further embodiment, an isolated peptide is provided consisting of an amino acid sequence X₁ X₂ X₃ X₄ X₅ L/V X₆ X₇ X₈ X₉ X₁₀ X₁₁ X₁₂ X₁₃ X₁₄, wherein each residue can be independently selected as follows (SEQ ID NO: 1):

X₁ is T, S, A or P;

X₂ is L, I, V, M, F, H, W or N;

X₃ is T, S, A, H, Y, F, W, N, D or E;

X₄ is R, Q, K, T, S or A;

X₅ is V, I, L, M, R, Q or K;

X₆ is T, S, A, Y, F, H, W, N, L, I, V or M

X₇ is D, E, V, L, I, M, F, Y, H, W or N;

X₈ is V, I, L, M, S, A, T, R, Q or K;

X₉ is Y, F, H, W, D, E, I, V, L, M or N;

X₁₀ is R, Q, K, W, Y, F, H, N, V, I, L, M or G;

X₁₁ is G, Y, F, H, W, N, S, A, T, I, V, L, M;

X₁₂ is R, Q, K, H, N, Y, F, W, I, V, L or M;

X₁₃ is L, V, I, M, T, S or A; and

X₁₄ is S, T, A, P, G, R, Q or K;

and variants, analogs and active fragments thereof

In a further embodiment, the peptide comprises an amino acid sequence F L H T R L X₁ X₂ X₃ X₄ X₅ X₆ X₇ X₈ X₉, wherein each residue can be independently selected as follows (SEQ ID NO: 2):

X₁ is T, S, A, Y, F, H, W, N, L, I, V or M;

X₂ is D, E, V, L, I, M, F, Y, H, W or N;

X₃ is V, I, L, M, S, A, T, R, Q or K;

X₄ is Y, F, H, W, D, E, I, V, L, M or N;

X₅ is R, Q, K, W, Y, F, H, N, V, I, L, M or G;

X₆ is G, Y, F, H, W, N, S, A, T, I, V, L, M;

X₇ is R, Q, K, H, N, Y, F, W, I, V, L or M;

X₈ is L, V, I, M, T, S or A; and

X₉ is S, T, A, P, G, R, Q or K;

and variants, analogs and active fragments thereof

More particularly, the peptide consists of an amino acid sequence F L H T R L X₁ X₂ X₃ X₄ X₅ X₆ X₇ X₈ X₉, wherein each residue can be independently selected as follows (SEQ ID NO: 2):

X₁ is T, S, A, Y, F, H, W, N, L, I, V or M;

X₂ is D, E, V, L, I, M, F, Y, H, W or N;

X₃ is V, I, L, M, S, A, T, R, Q or K;

X₄ is Y, F, H, W, D, E, I, V, L, M or N;

X₅ is R, Q, K, W, Y, F, H, N, V, I, L, M or G;

X₆ is G, Y, F, H, W, N, S, A, T, I, V, L, M;

X₇ is R, Q, K, H, N, Y, F, W, I, V, L or M;

X₈ is L, V, I, M, T, S or A; and

X₉ is S, T, A, P, G, R, Q or K;

and variants, analogs and active fragments thereof

In a still further embodiment, the peptide comprises an amino acid sequence F L H T R L F V X₁ X₂ X₃ X₄ X₅ X₆ X₇, wherein each residue can be independently selected as follows (SEQ ID NO: 3):

X₁ is V, I, L, M, S, A, T, R, Q or K;

X₂ is Y, F, H, W, D, E, I, V, L, M or N;

X₃ is R, Q, K, W, Y, F, H, N, V, I, L, M or G;

X₄ is G, Y, F, H, W, N, S, A, T, I, V, L, M;

X₅ is, Q, K, H, N, Y, F, W, I, V, L or M;

X₆ is L, V, I, M, T, S or A; and

X₇ is S, T, A, P, G, R, Q or K;

and variants, analogs and active fragments thereof

A peptide is provided consisting of an amino acid sequence F L H T R L F V X₁ X₂ X₃ X₄ X₅ X₆ X₇, wherein each residue can be independently selected as follows (SEQ ID NO: 3):

X₁ is V, I, L, M, S, A, T, R, Q or K;

X₂ is Y, F, H, W, D, E, I, V, L, M or N;

X₃ is R, Q, K, W, Y, F, H, N, V, I, L, M or G;

X₄ is G, Y, F, H, W, N, S, A, T, I, V, L, M;

X₅ is R, Q, K, H, N, Y, F, W, I, V, L or M;

X₆ is L, V, I, M, T, S or A; and

X₇ is S, T, A, P, G, R, Q or K;

and variants, analogs and active fragments thereof

In a further aspect, the peptide comprises an amino acid sequence X₁ X₂ X₃ X₄ X₅ L/V X₆ X₇ X₈ X₉ X₁₀ X₁₁ X₁₂ X₁₃ X₁₄, wherein each residue can be independently selected as follows (SEQ ID NO: 4):

X₁ is T or P;

X₂ is L or F;

X₃ is T, H or E;

X₄ is R or T;

X₅ is V or R;

X₆ is T, F or L;

X₇ is D, V or F;

X₈ is V, S or R;

X ₉ is Y, D, I or N;

X₁₀ is R, W, V or G;

X₁₁ is G, Y, S or I;

X₁₂ is R, H, N, Y or I;

X₁₃ is L, T or S; and

X₁₄ is S, P, G or R;

and variants, analogs and active fragments thereof

A further embodiment of a peptide of the present invention comprises an amino acid sequence F L H T R L X₁ X₂ X₃ X₄ X₅ X₆ X₇ X₈ X₉, wherein each residue can be independently selected as follows (SEQ ID NO: 5):

X₁ is T, F or L;

X₂ is D, V or F;

X₃ is V, S or R;

X₄ is Y, D, I or N;

X₅ is R, W, V or G;

X₆ is G, Y, S or I;

X₇ is R, H, N, Y or I;

X₈ is L, T or S; and

X₉ is S, P, G or R;

and variants, analogs and active fragments thereof

An additional embodiment of the peptide comprises an amino acid sequence F L H T R L F V X₁ X₂ X₃ X₄ X₅ X₆ X₇, wherein each residue can be independently selected as follows (SEQ ID NO 6):

X₁ is V, S or R;

X₂ is Y, D, I or N;

X₃ is R, W, V or G;

X₄ is G, Y, S or I;

X₅ is R, H, N, Y or I;

X₆ is L, T or S; and

X₇ is S, P, G or R;

and variants, analogs and active fragments thereof

In a particular embodiment, the peptide comprises the amino acid sequence set out in any of SEQ ID NOS: 27-38. Still further, the peptide comprises the amino acid sequence F L H T R L F V S D W Y H T (SEQ ID NO: 7). More particularly, the peptide comprises the amino acid sequence F L H T R L F V (SEQ ID NO: 8). Moreover, peptides having the amino acid sequence F L H T R L F V S D W Y H T (SEQ ID NO: 7) or F L H T R L F V (SEQ ID NO: 8) are provided. Still more particularly, the peptide comprises the amino acid sequence TRLFR V/F (SEQ ID NO: 39), FLHTRLFV (SEQ ID NO: 8), TRLF(R)V (SEQ ID NO: 40) or TRLF (SEQ ID NO: 41).

In a further embodiment, the present invention relates to certain therapeutic methods which would be based upon the activity of the carbohydrate epitope mimic peptide(s), variants, analogs or active fragments thereof, or upon agents or other compounds determined to possess the same activity. One such therapeutic method is associated with the prevention of the manifestations of conditions which can be corrected, altered or otherwise modulated by inhibition or activation of the binding activity of the carbohydrate epitope recognizing molecules, and comprises administering an agent capable of modulating the activity of the carbohydrate epitope recognizing molecules, either individually or in mixture with each other in an amount effective to prevent the development of those conditions in the host. In particular, binding partners to the carbohydrate epitope recognizing molecules, most particularly carbohydrate epitope mimic peptide(s), variants, analogs or active fragments thereof, may be administered to inhibit or potentiate the activity of carbohydrate epitope recognizing molecules. In a particular embodiment, an L2/HNK1 carbohydrate epitope mimic peptide may be administered to activate or otherwise modulate the activity of L2/HNK-1 recognizing molecules, as in the potentiation of neural cell adhesion molecules in CNS or PNS therapy.

More specifically, the therapeutic method generally referred to herein could include methods for the treatment of various pathologies or other cellular dysfunctions and derangements by the administration of pharmaceutical compositions that comprise the carbohydrate epitope mimic peptide(s), variants, analogs or active fragments thereof, effective inhibitors or enhancers of activation of the carbohydrate epitope mimic peptide(s), or other equally effective drugs developed for instance by a drug screening assay prepared and used in accordance with a further aspect of the present invention. For example, the carbohydrate epitope mimic peptide(s) of the present invention, variants, analogs or active fragments thereof, as particularly represented by any of SEQ ID NOS: 1-8, 27-38, 39, 40 and 41, may be administered to inhibit or potentiate activity of L2/HNK-1 carbohydrate epitope containing molecules or of L2/HNK-1 carbohydrate epitope recognizing molecules, as in the potentiation of neural cell adhesion molecules in CNS or PNS therapy.

It is a still further object of the present invention to provide a method for the treatment of mammals to control the amount or activity of the L2/HNK-1 carbohydrate epitope or L2/HNK-1 epitope containing molecules, so as to alter the adverse consequences of such presence or activity, or where beneficial, to enhance such activity.

It is a still further object of the present invention to provide a method for the treatment of mammals to control the amount or activity of L2/HNK-1 carbohydrate epitope recognizing molecules, so as to treat or avert the adverse consequences of invasive, spontaneous or idiopathic pathological states.

It is also an object of the present invention to provide method for promoting neural growth and/or remyelination and/or neuroprotection in vivo in the central nervous system of a mammal comprising administering to said mammal a neural growth and/or remyelination and/or neuroprotection promoting amount of the carbohydrate epitope mimic peptide(s) of the present invention, which peptide is capable of overcoming inhibitory molecular cues found on glial cells and myelin and promoting said neural growth; variants, analogs or active fragments thereof, antagonists thereof, antibodies thereto, and secreting or expressing cells thereof.

In a further embodiment, the invention provides a method of promoting neural growth and/or remyelination and/or neuroprotection in vivo in the central nervous system of a mammal comprising administering to said mammal a neural growth and/or remyelination and/or neuroprotection promoting amount of the carbohydrate epitope mimic peptide(s) of the present invention, variants, analogs or active fragments thereof, antagonists thereof, antibodies thereto, and secreting or expressing cells thereof, further comprising administering to said mammal a neural growth and/or remyelination and/or neuroprotection promoting amount of a neural cell adhesion molecule. In a particular embodiment, the neural cell adhesion molecule is selected from the group consisting of L1, N-CAM and myelin-associated glycoprotein. In a further particular embodiment, neural cell adhesion molecule is selected from the group consisting of laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

The present invention further relates to a method for promoting neural growth and/or remyelination and/or neuroprotection in vivo in the central nervous system of a mammal comprising administering to said mammal a neural growth promoting amount of an agent, said agent comprising a neural cell adhesion molecule, which molecule is capable of overcoming inhibitory molecular cues found on glial cells and myelin and promoting said neural growth, active fragments thereof, secreting cells thereof and soluble molecules thereof, said agent being modified by recombinant or chemical means to have the carbohydrate epitope mimic peptide(s) of the present invention, variants, analogs or active fragments thereof, attached thereto. In a particular embodiment of such method, the neural cell adhesion molecule is selected from the group consisting of L 1, N-CAM and myelin-associated glycoprotein. In a further particular embodiment of such method, the neural cell adhesion molecule is selected from the group consisting of laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

It is a further object to provide a method for enhancing memory, comprising administering to the brain of a mammal in need of such enhancement, an amount of the carbohydrate epitope mimic peptide(s) of the present invention, variants, analogs or active fragments thereof effective to enhance the memory of the mammal. In a particular embodiment, such a method further comprises administering to the brain of said mammal an amount of a neural cell adhesion molecule effective to enhance the memory of the mammal. In a particular embodiment, the method for enhancing memory comprises a method for inhibiting the onset or progression, or treating the presence or consequences of Alzheimers disease or dementia in a mammal.

It is an object of the present invention to provide a method for enhancing memory, comprising delivering to the cells of the brain of a mammal in need of such enhancement, a vector which allows for the expression of the carbohydrate epitope mimic peptide(s) of the present invention, variants, analogs or active fragments thereof In a particular embodiment, the method for enhancing memory comprises a method for inhibiting the onset or progression, or treating the presence or consequences of Alzheimers disease or dementia in a mammal.

In a further object, the present invention provides a method for increasing synaptic efficacy in the CNS of a mammal comprising administering to the brain of the mammal, an amount of the carbohydrate epitope mimic peptide(s) of the present invention, variants, analogs or active fragments thereof effective to increase synaptic efficacy in the brain of the mammal. In a particular embodiment, the increase in synaptic efficacy is demonstrated by the stabilization of long term potentiation.

In a still further object, the present invention provides a method of promoting neuroprotection and/or neuronal survival in a mammal comprising delivering to the cells of the brain of a mammal in need thereof, a vector which allows for the expression of the carbohydrate epitope mimic peptide(s) of the present invention, variants, analogs or active fragments thereof. In a particular embodiment, such a method comprises a method for inhibiting the development or onset, or treating the presence in a mammal of a condition selected from the group consisting of apoptosis, necrosis, Alzheimers disease, dementia, Parkinsons disease, multiple sclerosis, acute spinal cord injury, chronic spinal cord injury, any of the foregoing where neurodegeneration occurs or may occur, and combinations thereof.

In a further embodiment, the present invention provides a method for inhibiting axonal cell death and enhancing myelination and remyelination in the central nervous system of a mammal comprising administering to said mammal a therapeutically effective amount of the carbohydrate epitope mimic peptide(s) of the present invention, which peptide is capable of overcoming inhibitory molecular cues found on glial cells and myelin and promoting said neural growth, variants, analogs or active fragments thereof, antagonists thereof, antibodies thereto, and secreting or expressing cells thereof.

It is an object of the present invention to provide a method for preventing, ameliorating or blocking viral infection of a mammal comprising administering to said mammal an effective amount of the peptide of the present invention, variants thereof, analogs thereof, active fragments thereof or derivatives thereof. In a particular embodiment, the viral infection is the result of the human immunodeficiency virus.

In particular, the carbohydrate epitope mimic peptide(s) whose sequences are presented in SEQ ID NOS: 1-8, 27-38, 39, 40 and 41 herein, variants, analogs, derivatives, agonists, antagonists, or active fragments thereof, could be prepared in pharmaceutical formulations for administration in instances wherein therapy to activate, inhibit or otherwise modulate L2/HNK-1 carbohydrate-recognizing molecules is appropriate, such as to promote neural growth in CNS or PNS therapy and as otherwise recited hereinabove. The specificity of the carbohydrate epitope mimic peptide(s) hereof would make it possible to better manage the untoward effects of current CNS or PNS therapy, and would thereby make it possible to apply the carbohydrate epitope mimic peptide(s) as a general neural growth or neuroprotection promoting agent.

Accordingly, it is a principal object of the present invention to provide carbohydrate epitope mimic peptide(s), variants, analogs, derivatives or active fragments thereof, in purified form, that exhibits certain characteristics and activities associated with the L2/HNK-1 carbohydrate epitope or L2/HNK-1 carbohydrate epitope containing molecules for the promotion or modulation of the activity of L2/HNK-1 carbohydrate epitope recognizing molecules.

It is a still further object of the present invention to provide pharmaceutical compositions for use in therapeutic methods which comprise or are based upon the carbohydrate epitope mimic peptide(s), variants, analogs, derivatives or active fragments thereof, their binding partner(s), or upon agents or compounds that control the production, or that mimic or antagonize the activities of the L2/HNK-1carbohydrate epitope, all as aforesaid.

It is thus an object of the present invention to provide a pharmaceutical composition for the modulation of neural growth in the central nervous system of a mammal, comprising a therapeutically effective amount of the carbohydrate epitope mimic peptide(s) of the present invention, which peptide is capable of overcoming inhibitory molecular cues found on glial cells and myelin and promoting said neural growth, variants, analogs, derivatives or active fragments thereof, and secreting or expressing cells thereof, and a pharmaceutically acceptable carrier.

It is a further object to provide a pharmaceutical composition for promoting neural growth and/or remyelination and/or neuroprotection, comprising a therapeutically effective amount of a carbohydrate epitope mimic peptide(s), variants, analogs, derivatives or active fragments thereof, and secreting or expressing cells thereof, and a pharmaceutically acceptable carrier. In a particular embodiment, the pharmaceutical composition further comprises a therapeutically effective amount of a neural cell adhesion molecule. Still more particularly, the neural cell adhesion molecule is selected from the group consisting of L1, N-CAM and myelin-associated glycoprotein. In a further particular embodiment, neural cell adhesion molecule is selected from the group consisting of laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

It is an object of the present invention to provide a pharmaceutical composition for preventing, ameliorating or blocking viral infection comprising a therapeutically effective amount of the peptide of the present invention or variants, analogs, derivatives or active fragments thereof and a pharmaceutically acceptable carrier.

In a still further object, the invention encompasses derivatives of a carbohydrate epitope mimic peptide, including derivatives of variants, analogs or active fragments of such peptide. Such derivatives encompass and include derivatives to enhance activity, solubility, effective therapeutic concentration, and transport across the blood brain barrier. Further encompassed derivatives include the attachment of moieties or molecules which are known to contain the L2/HNK-1 carbohydrate epitope or which recognize the L2/HNK-1 carbohydrate epitope.

Such a derivative includes a derivative of the carbohydrate epitope mimic peptide(s) of the present invention, variants, analogs or active fragments thereof, capable of mimicking the carbohydrate epitope GlcAβ1→3Galβ1→4GlcNAc, having one or more chemical moieties attached thereto.

More particularly, a derivative in object includes a derivative wherein at least one of said chemical moieties is a water-soluble polymer capable of enhancing solubility of said peptide. Still more particular is a derivative wherein at least one of said chemical moeities is a molecule which facilitates transfer or transport across the blood brain barrier. A further and more particular object is to provide a derivative wherein said molecule is selected from the group consisting of a biocompatible hydrophobic molecule, transferrin, ApoE or ApoJ.

It is a further object of the present invention to provide a derivative wherein at least one of said chemical moieties is a molecule having multiple sites for peptide attachment and capable of binding at least two of said peptides simultaneously to generate a multimeric peptide structure. More particularly, such molecule is selected from the group of BSA, ovalbumin, human serum allbumin, polyacrylamide, beads and synthetic fibers (biodegradable and non-biodegradable).

It is a further object of the present invention to provide a derivative wherein at least one of said chemical moieties is a neural cell adhesion molecule. More particularly, the neural cell adhesion molecule is selected from the group consisting of L1, N-CAM and myelin-associated glycoprotein. In a further particular embodiment, neural cell adhesion molecule is selected from the group consisting of laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

It is an object to provide a derivative wherein at least one of said chemical moieties is a branched or unbranched polymer.

It is a further object to provide any of such derivatives wherein at least one of said chemical moieties is N-terminally attached to said polypeptide. In a further embodiment, at least one of said chemical moieties is C-terminally attached to said polypeptide.

The present invention also relates to nucleic acid sequences, or degenerate variants thereof, which encode a carbohydrate epitope mimic peptide, particularly a peptide capable of mimicking the L2/HNK-1 carbohydrate epitope. Particularly preferred is a nucleic acid molecule, in particular a recombinant DNA molecule, encoding the L2/HNK-1 carbohydrate epitope mimic peptide, which in a particular embodiment comprises a nucleotide sequence capable of encoding the peptide set out in any of SEQ ID NOs: 1-8, 27-38, 39, 40 or 41 or which is complementary to such a nucleotide sequence. Thus, in a preferred embodiment, a recombinant DNA molecule (or its complement) is provided which encodes the peptide set out in any of SEQ ID NOs: 1-8, 27-38, 39, 40 or 41. Particular examples of such a DNA sequence or recombinant DNA molecule, capable of encoding the peptide F L H T R L F V S D W Y H T (SEQ ID NO: 7), are provided in SEQ ID NOS: 9-20. Further particular examples of such a DNA sequence or recombinant DNA molecule, capable of encoding the peptide F L H T R L F V (SEQ ID NO: 8), are provided in SEQ ID NOS: 21-26. Examples of such a DNA sequence or recombinant DNA molecule, capable of encoding the peptide TRLFR V/F (SEQ ID NO: 39) are provided in SEQ ID NOS: 42-44 and examples capable of encoding the peptide TRLF(R)V (SEQ ID NO: 40) are provided in SEQ ID NOS: 45-47. Still further particular examples of such a DNA sequence or recombinant DNA molecule, capable of encoding the peptide TRLF (SEQ ID NO: 41) are provided in SEQ ID NOS: 48-50.

The DNA sequences of the carbohydrate epitope mimic peptide(s) of the present invention or portions thereof, may be prepared as probes to screen for complementary sequences. The present invention extends to probes so prepared that may be provided for screening phage, cDNA and genomic libraries for the carbohydrate epitope mimic peptide(s). For example, the probes may be prepared with a variety of known vectors, such as the phage λ vector. The present invention also includes the preparation of plasmids including such vectors, and the use of the DNA sequences to construct vectors expressing antisense RNA or ribozymes which would attack the mRNAs of any or all of the DNA sequences which are capable of encoding the peptide set out in any of SEQ ID NOS: 1-8, 27-38, 39, 40 and 41. Correspondingly, the preparation of antisense RNA and ribozymes are included herein.

In a further embodiment of the invention, the full DNA sequence of the recombinant DNA molecule may be operatively linked to an expression control sequence which may be introduced into an appropriate host. The invention accordingly extends to unicellular hosts transformed with the recombinant DNA molecule comprising a DNA sequence encoding the present carbohydrate epitope mimic peptide(s), and more particularly, a complete DNA sequence which is capable of encoding the peptide set out in any of SEQ ID NOS: 1-8, 27-38, 39, 40 and 41.

It is therefore an object of the present invention to provide a DNA sequence which encodes a carbohydrate epitope mimic peptide, including variants, analogs and active fragments thereof It is a further object of the present invention to provide a DNA sequence which encodes a carbohydrate epitope mimic peptide, including variants, analogs and active fragments thereof, selected from the group consisting of:

(A) DNA capable of encoding the peptide set out in any of SEQ ID NOS: 1-8, 27-38, 39, 40 and 41;

(B) DNA sequences that hybridize to any of the foregoing DNA sequences under standard hybridization conditions; and

(C) DNA sequences that code on expression for an amino acid sequence encoded by any of the foregoing DNA sequences.

The present invention naturally contemplates several means for preparation of the carbohydrate epitope mimic peptide, including as illustrated herein known peptide synthesis and recombinant techniques, and the invention is accordingly intended to cover such synthetic preparations within its scope. The nucleic acid and amino acid sequences disclosed herein facilitates the reproduction of the carbohydrate epitope mimic peptide, including variants, analogs and active fragments thereof, by such recombinant techniques, and accordingly, the invention extends to expression vectors prepared from the disclosed DNA sequences for expression in host systems by recombinant DNA techniques, and to the resulting transformed hosts.

It is a still further object to provide a recombinant DNA molecule comprising a DNA sequence or degenerate variant thereof and a heterologous nucleotide sequence, wherein said DNA sequence or degenerate variant encodes a carbohydrate epitope mimic peptide, including variants, analogs and active fragments thereof, selected from the group consisting of:

(A) DNA capable of encoding the peptide set out in any of SEQ ID NOS: 1-8, 27-38, 39, 40 and 41;

(B) DNA sequences that hybridize to any of the foregoing DNA sequences under standard hybridization conditions; and

(C) DNA sequences that code on expression for an amino acid sequence encoded by any of the foregoing DNA sequences.

In a particular embodiment of the recombinant DNA molecule, said DNA sequence is operatively linked to an expression control sequence. In a further particular embodiment, said expression control sequence is selected from the group consisting of the early or late promoters of SV40 or adenovirus, the lac system, the trp system, the TAC system, the TRC system, the major operator and promoter regions of phage λ, the control regions of fd coat protein, the promoter for 3-phosphoglycerate kinase, the promoters of acid phosphatase and the promoters of the yeast α-mating factors, the promoters of neural cell adhesion molecules, the promoter of L1, the gFAP promoter and the promoter of myelin basic protein.

The invention also provides a unicellular host transformed with a recombinant DNA molecule comprising a DNA sequence or degenerate variant thereof, which encodes a carbohydrate epitope mimic peptide, including variants, analogs and active fragments thereof, selected from the group consisting of:

(A) DNA capable of encoding the peptide set out in any of SEQ ID NOS: 1-8, 27-38, 39, 40 and 41;

(B) DNA sequences that hybridize to any of the foregoing DNA sequences under standard hybridization conditions; and

(C) DNA sequences that code on expression for an amino acid sequence encoded by any of the foregoing DNA sequences;

wherein said DNA sequence is operatively linked to an expression control sequence.

In a further embodiment, the unicellular host is selected from the group consisting of E. coli, Pseudomonas, Bacillus, Streptomyces, yeasts, CHO, R1.1, B-W, L-M, COS 1, COS 7, BSC1, BSC40, and BMT10 cells, plant cells, insect cells, mammalian cells, human cells and neural cells in tissue culture.

Still further provided is a cloning vector which comprises the DNA sequence encoding a carbohydrate epitope mimic peptide, including variants, analogs and active fragments thereof, and a heterologous nucleotide sequence.

According to other preferred features of certain preferred embodiments of the present invention, a recombinant expression system is provided to produce biologically active carbohydrate epitope mimic peptide, including variants, analogs and active fragments thereof.

It is therefore an object to provide an expression vector which comprises a DNA sequence encoding a carbohydrate epitope mimic peptide, including variants, analogs and active fragments thereof, and a heterologous nucleotide sequence. In a particular embodiment, the heterologous nucleotide sequence is an expression control sequence.

In a more particular embodiment, the heterologous nucleotide sequence encodes a neural cell adhesion molecule.

The invention includes an assay system for screening of potential drugs effective to modulate L2/HNK-1 carbohydrate epitope recognizing activity of target mammalian cells by mimicking, interrupting or potentiating the interaction or recognition of the L2/HNK-1 carbohydrate epitope. In one instance, the test drug could be administered to a cellular sample with the L2/HNK-1 carbohydrate epitope recognizing molecule, or an extract containing the carbohydrate epitope mimic peptide, to determine its effect upon the binding activity of the L2/HNK-1 carbohydrate epitope recognizing molecule, by comparison with a control.

The assay system could more importantly be adapted to identify drugs or other entities that are capable of binding to the L2/HNK-1 carbohydrate epitope recognizing molecule, thereby inhibiting or potentiating the activity of the carbohydrate epitope mimic peptide. Such assay would be useful in the development of drugs that would be specific against particular cellular activity, or that would potentiate such activity, in time or in level of activity.

In yet a further embodiment, the invention contemplates antagonists of the activity of a carbohydrate epitope mimic peptide. In particular, an agent or molecule that inhibits the carbohydrate epitope mimic peptide or blocks its interaction with an L2/HNK-1 carbohydrate epitope recognizing molecule.

It is a further object of the present invention to provide a method and associated assay system for screening substances such as drugs, agents and the like, potentially effective in either mimicking the activity or combating the adverse effects of the carbohydrate epitope mimic peptide in mammals.

It is thus an object of this invention to provide a method for detecting the presence or activity of a peptide or compound, said peptide or compound capable of mimicking the carbohydrate epitope GlcAβ1→3Galβ1→4GlcNAc or sulfate -3GlcAβ1→3Galβ1→4GlcNAc wherein said peptide or compound is measured by:

A. contacting a sample in which the presence or activity of said peptide or compound is suspected with a binding partner of said peptide or compound under conditions that allow binding of said peptide or compound to said binding partner to occur; and

B. detecting whether binding has occurred between said peptide or compound from said sample and the binding partner;

wherein the detection of binding indicates that presence or activity of said peptide or compound in said sample.

In a particular embodiment of such method, the binding partner is selected from the group consisting of an antibody which recognizes GlcAβ1→3Galβ1→4GlcNAc; an antibody which recognizes sulfate -3GlcAβ1→3Galβ1→4GlcNAc; L2-412 antibody; HNK-1 antibody; a polypeptide molecule which binds or otherwise interacts with GlcAβ1→3Galβ1→4GlcNAc or sulfate -3GlcAβ1→3Galβ1→4GlcNAc; laminin; P-selectin; L-selectin; and a neural cell adhesion molecule.

Further provided is a method of testing the ability of a drug or other entity to mimic the carbohydrate epitope GlcAβ1→3Galβ1→4GlcNAc or sulfate -3GlcAβ1→3Galβ1→4GlcNAc which comprises:

a. adding CNS neurons to a cell culture system;

b. adding the drug or other entity under test to the cell culture system;

c. measuring the neuronal outgrowth of the CNS neurons; and

d. correlating a difference in the level of neuronal outgrowth of cells in the presence of the drug relative to a control culture to which no drug is added to the ability of the drug to mimic the carbohydrate epitope GlcAβ1→3Galβ1→4GlcNAc or sulfate -3GlcAβ1→3Galβ1→4GlcNAc.

The diagnostic utility of the present invention extends to the use of the present carbohydrate epitope mimic peptide in assays to screen for L2/HNK-1 carbohydrate epitope recognizing molecules. Thus, the carbohydrate epitope mimic peptide(s), including variants, analogs and active fragments thereof, and any antagonists or antibodies that may exist or be raised thereto, are capable of use in connection with various diagnostic techniques, including immunoassays, such as a radioimmunoassay, using for example, an antibody to the carbohydrate epitope mimic peptide that has been labeled by either radioactive addition, or radioiodination.

In an immunoassay, a control quantity of the antagonists or antibodies thereto, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

The present invention includes an assay system which may be prepared in the form of a test kit for the quantitative analysis of the extent of the presence of the carbohydrate epitope mimic peptide, or to identify drugs or other agents that may mimic or block their activity. The system or test kit may comprise a labeled component prepared by one of the radioactive and/or enzymatic techniques discussed herein, coupling a label to the carbohydrate epitope mimic peptide, their agonists and/or antagonists, and one or more additional immunochemical reagents, at least one of which is a free or immobilized ligand, capable either of binding with the labeled component, its binding partner, one of the components to be determined or their binding partner(s).

The invention thus provides a test kit for the demonstration of a molecule capable of binding GlcAβ1→3Galβ1→4GlcNAc or sulfate -3GlcAβ1→3Galβ1-4GlcNAc in a eukaryotic cellular sample, comprising:

A. a predetermined amount of a detectably labeled compound or peptide, said peptide or compound capable of mimicking the carbohydrate epitope GlcAβ1→3Galβ1→4GlcNAc or sulfate GlcAβ1→3Galβ1→4GlcNAc;

B. other reagents; and

C. directions for use of said kit.

The invention further provides a test kit for demonstrating the presence of a molecule capable of binding 3GlcAβ1→3Galβ1→4GlcNAc or sulfate -3GlcAβ1→3Galβ1→4GlcNAc in a eukaryotic cellular sample, comprising:

A. a predetermined amount of a compound or peptide, said peptide or compound capable of mimicking the carbohydrate epitope GlcAβ1→3Galβ1→4GlcNAc or sulfate -3GlcAβ1→3Galβ1→4GlcNAc;

B. a predetermined amount of a specific binding partner of said compound or peptide;

C. other reagents; and

D. directions for use of said kit;

wherein either said compound or peptide or said specific binding partner are detectably labeled.

The present invention likewise extends to the development and use of antibodies against the carbohydrate epitope mimic peptide(s), including naturally raised and recombinantly prepared antibodies. Such antibodies could include both polyclonal and monoclonal antibodies prepared by known genetic techniques, as well as bi-specific (chimeric) antibodies, and antibodies including other functionalities suiting them for additional diagnostic use conjunctive with their capability of modulating carbohydrate epitope mimic peptide activity. It is a further object of the present invention to provide antibodies to the carbohydrate epitope mimic peptide, including variants, analogs and active fragments thereof, and methods for their preparation, including recombinant means.

Other objects and advantages will become apparent to those skilled in the art from a review of the following description which proceeds with reference to the following illustrative drawings.

### BRIEF DESCRIPTION OF THE DRAWINGS

FIG. 1 is a flow diagram of the phage library screening. The library was screened in three cycles, or rounds, of panning with the antibody L2-412 or the antibody HNK-1.

FIG. 2A depicts competition of the L2-412 antibody to immobilized L2/HNK-1 glycolipids by various inhibitors: positive phage (denoted phage 15-15), negative phage (denoted neg. control phage), free peptide (denoted peptide 15-15) and SO₃-sugar. L2-412 was preincubated with a stepwise 2-fold dilution series of the free peptide (starting concentration 2.2 mM), SO₃-sugar (starting concentration 5 mM), positive phage and negative phage (starting concentration 10¹²TUs/ml), and added to the coated glycolipid. After incubation and washing, the bound antibody was detected with HRP anti-rat antibody.

FIG. 2B depicts the percentage inhibition of L2-412 antibody to immobilized L2/HNK-1 glycolipids by various inhibitors. The percentage is calculated for the point in FIG. 2A with the highest concentration of inhibitor. The binding of L2-412 in the absence of inhibitor is defined as 0% inhibition. Mean+/−values standard deviation from 4 experiments carried out in duplicate.

FIG. 3 Depicts competition of positive phage binding to immobilized L2-412 with the 15-15 peptide coupled to BSA.

FIG. 4 depicts competition of positive phage binding to immobilized laminin with the 15-15 peptide coupled to BSA.

FIG. 5 depicts binding of phage 15-15 and control phage UBR2 to immobilized laminin (100 μl of 10 μg/ml used for coating). Bound phage were detected by HRP-conjugated anti-M13 antibody. The results are presented as OD₄₀₅ vs. relative concentration of the phage preparation (a relative concentration of 100 corresponds to 1012 TUs/ml phage).

FIG. 6 depicts the binding of biotinylated peptide -BSA to L2-412 in a concentration-dependent manner. biot BSA is the control biotinylated BSA.

FIG. 7 depicts the binding of biotinylated-peptide-BSA to immobilized laminin in a concentration-dependent manner. biot BSA is the control biotinylated BSA.

FIG. 8 is a diagramatic representation of outgrowth of neurites from chick motor neurons on substrate consisting of collagen mixed with the peptide-BSA conjugates or BSA as control.

FIGS. 9A-9C shows outgrowth of neurites from motor neurons (network) cultured on substrate consisting of: (A) 8 amino acid peptide coupled to BSA, 15 amino acid peptide coupled to BSA; (B) scrambled 8 amino acid peptide coupled to BSA, scrambled 15 amino acid peptide coupled to BSA; and (C) BSA. The bar represents 20 μm.

FIG. 10 depicts the average length of the longest neurite and average length of all neurites when cultured in the presence of: the 8 amino acid peptide; the L2-HNK-1glycolipid; the 15 amino acid peptide; the scrambled 8 amino acid peptide; the scrambled 15 amino acid peptide; and BSA.

FIG. 11 depicts the degree of polarity, calculated as the ratio of the mean length of the longest neurite divided by the average length of all neurites, of motor neurons cultured in the presence of: 8 amino acid peptide; L2/HNK-1 glycolipid; 15 amino acid peptide; scrambled 8 amino acid peptide; scrambled 15 amino acid peptide; and BSA.

FIGS. 12A-12F depicts the outgrowth of neurites from dorsal root ganglion neurons cultured on substrate consisting of: (A) BSA; (B) 8 amino acid peptide coupled to BSA; (C) 15 amino acid peptide coupled to BSA; (D) scrambled 15 amino acid peptide coupled to BSA; (E) BSA; and (F)L2/HNK-1 glycolipid. The bar represents 20 μm.

FIGS. 13A-13C shows staining of motor neurons by: (A) biotinylated 8 amino acid peptide coupled to BSA; (B) biotinylated scrambled 8 amino acid peptide coupled to BSA; and (C) biotinylated BSA. Detection was done with streptavidin-HRP. The bar represents 20 μm.

FIG. 14 depicts binding of HNK-1 selected phage 15H92 and 15H233, L2-412 selected 15-15 phage, and controls UBR2 and UBH to bound L2-412 antibody, IgG, HNK-1 antibody, and IgM. Detection was done with HRP-coupled anti-phage antibody.

FIG. 15 depicts comparative binding of various phage clones to bound antibody L2-412 and antibody HNK-1. L2-412 selected phage clones are 15-90, 15-91, 15-92, 15-93, 15-94 and 15-95. HNK-1 selected phage clones are 15H92, 15H94, 15H86, 15H85, 15H78, 15H36, 15H34 and 15H26. K91Kan, UB412 and UB HNK-1 are controls. Detection was done with HRP-coupled anti-phage antibody.

FIG. 16 depicts comparative binding of various phage clones to bound antibody L2-412 and antibody HNK-1. L2-412 selected phage clones are 15cho4, 15-94, 15-15 and 15ph1. HNK-1 selected clones are 15H212, 15H207, 15H208, 15H26, 15H78, 15H233, 15H136 and 15H92. UBR2 and UBH are unbound phage controls. Detection was done with HRP-coupled anti-phage antibody.

FIG. 17 depicts comparative binding of phage 15-15, 15H92 and unbound phage UBR2 and UBH to bound antibodies L2-412 and HNK-1. Detection was done with HRP-coupled anti-phage antibody. The vertical axis indicating absorbance at OD 405 nm. The sequences of the 15-mer phage inserts of 15-15 (SEQ ID NO: 28) and 15H92 (SEQ ID NO: 34) are also shown, with the homologous (consensus) amino acids in bold.

FIG. 18 depicts L2 glycolipid binding to CD4 peptide in a concentration-dependent manner.

FIG. 19. depicts competition of L2 glycolipid binding to immobilized laminin with the CD4 peptide.

FIG. 20 depicts fluorescence microscopy of cultures treated with gp120 alone or with HNK-1 epitope mimic peptide.

A (Upper left hand panel): Culture was not treated with either the HNK-1 epitope mimic peptide or gp120. RIP positive oligodendrocytes were observed in control wells, but only minimal membrane deposition onto the substrate was seen.

B (Upper right hand panel): Culture was treated with 10 nM HNK-1 epitope mimic peptide. Numerous mature RIP positive oligodendrocytes with extensive membrane sheaths were observed.

C (Bottom left hand panel): Culture was treated with 1 nM gp120. Mature RIP positive oligodendrocytes with intact sheaths of membrane were not observed. The only RIP positive oligodendrocytes observed in these cultures were immature oligodendrocytes, lacking membrane sheaths and RIP positive oligodendrocytes with collapsed processes, i.e., degenerating mature oligodendrocytes.

D (Bottom right hand panel): Culture was treated with 1 nM gp120 that was preincubated with 1 uM HNK-1 epitope mimic peptide. Mature RIP positive cells were indistinguishable from mature RIP positive cells observed in cultures treated with the HNK-1 epitope mimic peptide only. Oligodendrocytes were observed elaborating extensive sheaths of membrane.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The present invention encompasses carbohydrate epitope mimic peptides, which peptides mimic the structure and/or activity of carbohydrate epitopes. The present invention is particularly exemplified in L2/HNK1 carbohydrate epitope mimic peptides, capable of mimicking the structure and/or activity of the L2 and/or HNK1 epitope, particularly the carbohydrate epitope GlcAβ1→3Galβ1→4GlcNAc or sulfate -3GlcAβ1→3Galβ1→4GlcNAc. The carbohydrate epitope mimic peptides mimic or can otherwise replace, interact with, block, or facilitate particular carbohydrate epitopes which participate in carbohydrate-protein and protein-protein interactions. These carbohydrate epitopes and carbohydrate epitope containing molecules interact with themselves and/or carbohydrate epitope recognizing molecules.

The present invention provides L2/HNK1 carbohydrate epitope mimic peptides which particularly mimic the carbohydrate epitope GlcAβ1→3Galβ1→4GlcNAc or sulfate -3GlcAβ1→3Galβ1→4GlcNAc. L2/HNK1 carbohydrate epitope mimic peptides comprising the amino acid sequences set out in any of SEQ ID NOS:1-8, 27-38, 39, 40 and 41 are provided herein.

If appearing herein, the following terms shall have the definitions set out below.

The terms “carbohydrate epitope mimic peptide(s)”, “carbohydrate epitope mimic” “carbohydrate epitope peptidomimetic” and “peptidomimetic” and any variants not specifically listed, may be used herein interchangeably, and as used throughout the present application and claims refer to proteinaceous material including peptides which mimic the structure of a carbohydrate epitope, thereby mimicking, modulating or otherwise facilitating the activity of the carbohydrate epitope or ligand. Carbohydrate epitope mimic peptide(s) are particularly exemplified herein in the peptides of the present invention which mimic the carbohydrate epitope GlcAβ1→3Galβ1→4.GlcNAc or sulfate -3GlcAβ1→3Galβ1→4GlcNAc. The carbohydrate epitope mimic peptide(s) particularly exemplified herein mimic the L2/HNK1 epitope and comprise peptides having the amino acid sequences described herein and presented in SEQ ID NOS: 1-8, 39, 40 and 41 and in TABLE 2 and TABLE 4, and the profile of activities and characteristics set forth herein and in the claims. The terms “carbohydrate epitope mimic peptide(s)”, “carbohydrate epitope mimic”, “carbohydrate epitope peptidomimetic” and “peptidomimetic” are intended to include within their scope those peptides specifically recited herein as well as all variants, analogs and active fragments thereof, including substantially homologous variants and analogs.

The terms “L2/HNK1 carbohydrate epitope mimic peptide(s)”, “L2/HNK1 epitope mimic peptide(s)”, “L2 epitope mimic peptide(s)”, “HNK1 epitope mimic peptide(s)”, “L2/HNK1 peptidomimetic(s)”, and any variants not specifically listed, may be used herein interchangeably, and as used throughout the present application and claims refer to proteinaceous material including peptides, and extends to those peptides having the amino acid sequences described herein and presented in SEQ ID NOS: 1-8, 39, 40 and 41 and in TABLE 2 and TABLE 4, and the profile of activities and characteristics set forth herein and in the claims. Accordingly, peptides displaying substantially equivalent or altered activity are likewise contemplated. These modifications may be deliberate, for example, such as modifications obtained through site-directed mutagenesis, or may be accidental, such as those obtained through screening for carbohydrate epitope mimic peptide(s) using the methods and assays provided and described herein. Also, the terms “L2/HNK1 carbohydrate epitope mimic peptide(s)”, “L2/HNK1 epitope mimic peptide(s)”, “L2 epitope mimic peptide(s)”, “HNK1 epitope mimic peptide(s)”, “L2/HNK1 peptidomimetic(s)” are intended to include within their scope those peptides specifically recited herein as well as all variants, analogs and active fragments thereof, including substantially homologous variants and analogs.

The identity or location of one or more amino acid residues may be changed or modified to include, for example, active fragments such as deletions containing less than all of the residues specified for the peptide, variants wherein one or more residues are replaced or substituted by other residues or wherein one or more amino acid residues are added to a terminal or medial portion of the peptide, and analogs wherein one or more residues are replaced or substituted with unnatural amino acids, L-amino acids, various “designer” amino acids (for example β-methyl amino acids, Cαmethyl amino acids, and Nα-methyl amino acids), nonclassical amino acids or synthetic amino acids. Analogs further encompass cyclic peptides, which can be generated by any of recognized methods in the art.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

Synthetic peptide, prepared using the well known techniques of solid phase, liquid phase, or peptide condensation techniques, or any combination thereof, can include natural and unnatural amino acids. Amino acids used for peptide synthesis may be standard Boc (N^{α}-amino protected N^{α}-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile Nα-amino protected 9-fluorenylmethoxycarbonyl (Fmoc) amino acids first described by Carpino and Han (1972, J. Org. Chem. 37:3403-3409). Thus, polypeptide of the invention may comprise D-amino acids, a combination of D- and L-amino acids, and various “designer” amino acids (e.g., P-methyl amino acids, Cα-methyl amino acids, and Nα-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, α-helices, β turns, β sheets, γ-turns, and cyclic peptides can be generated.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

In one aspect of the invention, the peptides may comprise a special amino acid at the C-terminus which incorporates either a CO₂H or CONH₂ side chain to simulate a free glycine or a glycine-amide group. Another way to consider this special residue would be as a D or L amino acid analog with a side chain consisting of the linker or bond to the bead. In one embodiment, the pseudo-free C-terminal residue may be of the D or the L optical configuration; in another embodiment, a racemic mixture of D and L-isomers may be used.

In an additional embodiment, pyroglutamate may be included as the N-terminal residue of the peptide. Although pyroglutamate is not amenable to sequence by Edman degradation, by limiting substitution to only 50% of the peptides on a given bead with N-terminal pyroglutamate, there will remain enough non-pyroglutamate peptide on the bead for sequencing. One of ordinary skill would readily recognize that this technique could be used for sequencing of any peptide that incorporates a residue resistant to Edman degradation at the N-terminus. Other methods to characterize individual peptides that demonstrate desired activity are described in detail infra. Specific activity of a peptide that comprises a blocked N-terminal group, e.g., pyroglutamate, when the particular N-terminal group is present in 50% of the peptides, would readily be demonstrated by comparing activity of a completely (100%) blocked peptide with a non-blocked (0%) peptide.

In addition, the present invention envisions preparing peptides that have more well defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such peptides would provide ligands with unique function and activity, such as extended half-lives in vivo due to resistance to metabolic breakdown, or protease activity. Furthermore, it is well known that in certain systems constrained peptides show enhanced functional activity (Hruby, 1982, Life Sciences 31:189-199; Hruby et al., 1990, Biochem J. 268:249-262); the present invention provides a method to produce a constrained peptide that incorporates random sequences at all other positions.

A constrained, cyclic or rigidized peptide may be prepared synthetically, provided that in at least two positions in the sequence of the peptide an amino acid or amino acid analog is inserted that provides a chemical functional group capable of cross-linking to constrain, cyclise or rigidize the peptide after treatment to form the cross-link. Cyclization will be favored when a turn-inducing amino acid is incorporated. Examples of amino acids capable of cross-linking a peptide are cysteine to form disulfide, aspartic acid to form a lactone or a lactase, and a chelator such as γ-carboxyl-glutamic acid (Gla) (Bachem) to chelate a transition metal and form a cross-link. Protected γ-carboxyl glutamic acid may be prepared by modifying the synthesis described by Zee-Cheng and Olson (1980, Biophys. Biochem. Res. Commun. 94:1128-1132). A peptide in which the peptide sequence comprises at least two amino acids capable of cross-linking may be treated, e.g., by oxidation of cysteine residues to form a disulfide or addition of a metal ion to form a chelate, so as to cross-link the peptide and form a constrained, cyclic or rigidized peptide.

The present invention provides strategies to systematically prepare cross-links. For example, if four cysteine residues are incorporated in the peptide sequence, different protecting groups may be used (Hiskey, 1981, in The Peptides: Analysis, Synthesis, Biology, Vol. 3, Gross, and Meienhofer, eds., Academic Press: New York, pp. 137-167; Ponsanti et al., 1990, Tetrahedron 46:8255-8266). The first pair of cysteine may be deprotected and oxidized, then the second set may be deprotected and oxidized. In this way a defined set of disulfide cross-links may be formed. Alternatively, a pair of cysteine and a pair of collating amino acid analogs may be incorporated so that the cross-links are of a different chemical nature.

The following non-classical amino acids may be incorporated in the peptide in order to introduce particular conformational motifs: 1,2,3,4-tetrahydroisoquinoline-3-carboxylate (Kazmierski et al., 1991, J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby, 1991, Tetrahedron Lett.); 2-aminotetrahydronaphthalene-2-carboxylic acid (Landis, 1989, Ph.D. Thesis, University of Arizona); hydroxy-1,2,3,4-tetrahydroisoquinoline-3 carboxylate (Miyake et al., 1989, J. Takeda Res. Labs. 43:53-76); β-carboline (D and L) (Kazmierski, 1988, Ph.D. Thesis, University of Arizona); HIC (histidine isoquinoline carboxylic acid) (Zechel et al., 1991, Int. J. Pep. Protein Res. 43); and HIC (histidine cyclic urea) (Dharanipragada).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al., 1985, J. Org. Chem. 50:5834-5838); β-sheet inducing analogs (Kemp et al., 1988, Tetrahedron Left. 29:5081-5082); βturn inducing analogs (Kemp et al., 1988, Tetrahedron Lett. 29:5057-5060); ∝-helix inducing analogs (Kemp et al., 1988, Tetrahedron Left. 29:4935-4938), γ-turn inducing analogs (Kemp et al., 1989, J. Org. Chem. 54:109:115); and analogs provided by the following references: Nagai and Sato, 1985, Tetrahedron Lett. 26:647-650; DiMaio et al., 1989, J. Chem. Soc. Perkin Trans. p. 1687; also a Gly-Ala turn analog (Kahn et al., 1989, Tetrahedron Lett. 30:2317); amide bond isostere (Jones et al., 1988, Tetrahedron Lett. 29:3853-3856); tretrazol (Zabrocki et al., 1988, J. Am. Chem. Soc. 110:5875-5880); DTC (Samanen et al., 1990, Int. J. Protein Pep. Res. 35.:501-509), and analogs taught in Olson et al., 1990, J. Am. Chem. Sci. 112:323-333 and Garvey et al., 1990, J. Org. Chem. 56:436. Conformationally restricted mimetics of beta turns and beta bulges, and peptides containing them, are described in U.S. Pat. No. 5,440,013, issued Aug. 8, 1995 to Kahn.

The present invention further provides for modification or derivatization of the polypeptide or peptide of the invention. Modifications of peptides are well known to one of ordinary skill, and include phosphorylation, carboxymethylation, and acylation. Modifications may be effected by chemical or enzymatic means. In another aspect, glycosylated or fatty acylated peptide derivatives may be prepared. Preparation of glycosylated or fatty acylated peptides is well known in the art. Fatty acyl peptide derivatives may also be prepared. For example, and not by way of limitation, a free amino group (N-terminal or lysyl) may be acylated, e.g., myristoylated. In another embodiment an amino acid comprising an aliphatic side chain of the structure —(CH₂)ₙCH₃ may be incorporated in the peptide. This and other peptide-fatty acid conjugates suitable for use in the present invention are disclosed in U.K. Patent GB-8809162.4, International Patent Application PCT/AU89/00166, and reference 5, supra.

Chemical Moieties For Derivatization. Derivatives of the peptides (including variants, analogs and active fragments thereof) of the present invention are further provided. Such derivatives encompass and include derivatives to enhance activity, solubility, effective therapeutic concentration, and transport across the blood brain barrier. Further encompassed derivatives include the attachment of moieties or molecules which are known to contain the L2/HNK-1 carbohydrate epitope or which recognize the L2/HNK-1 carbohydrate epitope. The chemical moieties may be N-terminally or C-terminally attached to the peptides of the present invention. Chemical moieties suitable for derivatization may be, for instance, selected from among water soluble polymers. The polymer selected can be water soluble so that the component to which it is attached does not precipitate in an aqueous environment, such as a physiological environment. Preferably, for therapeutic use of the end-product preparation, the polymer will be pharmaceutically acceptable. The polymer may be branched or unbranched. One skilled in the art will be able to select the desired polymer based on such considerations as whether the polymer/component conjugate will be used therapeutically, and if so, the desired dosage, circulation time, resistance to proteolysis, and other considerations. For the present component or components, these may be ascertained using the assays provided herein.

The water soluble polymer may be selected from the group consisting of, for example, polyethylene glycol, copolymers of ethylene glycoupropylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols and polyvinyl alcohol. Polyethylene glycol propionaldenhyde may have advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The number of polymer molecules so attached may vary, and one skilled in the art will be able to ascertain the effect on function. One may mono-derivative, or may provide for a di-, tri-, tetra- or some combination of derivatization, with the same or different chemical moieties (e.g., polymers, such as different weights of polyethylene glycols). The proportion of polymer molecules to component or components molecules will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

The polyethylene glycol molecules (or other chemical moieties) should be attached to the component or components with consideration of effects on functional or antigenic domains of the protein. There are a number of attachment methods available to those skilled in the art, e.g., EP 0 401 384 herein incorporated by reference (coupling PEG to G-CSF), see also Malik et al., 1992, Exp. Hematol. 20:1028-1035 (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group include lysine residues and the—terminal amino acid residues; those having a free carboxyl group include aspartic acid residues glutamic acid residues and the C-terminal amino acid residue. Sulfhydrl groups may also be used as a reactive group for attaching the polyethylene glycol molecule(s). Preferred for therapeutic purposes is attachment at an amino group, such as attachment at the N-terminus or lysine group.

The invention provides derivatives wherein at least one of said attached chemical moieties is a molecule which facilitates transfer or transport across the blood-brain barrier, particularly molecules that naturally cross the blood-brain barrier. Examples of such molecules include a biocompatible hydrophobic molecule, transferrin or apolipoprotein. Transferrin has been shown to facilitate transfer, even or larger peptides, as for example, nerve growth factor (Friden, P. M. et al., Science 259, 373-377 (1993), Kordower, J. H. et al., Proc Natl Acad Sci USA 91, 9077-9080 (1994)). Apolipoprotein E (ApoE) and apolipoproetin J (ApoJ) have been shown to facilitate brain uptake of Alzheimer's amyloid beta protein when complexed thereto (Zlokovic, B. V. et al., Biochem. Biophys. Res. Commun. 205 (2), 1431-1437 (1994); Martel, C. L. et al., J. Neurochem 69(5), 1995-2004 (1997)).

More particularly the present invention provides derivatives which are fusion proteins comprising the peptides of the present invention or fragments thereof Thus peptides of the present invention and fragments thereof can be “modified” i.e., placed in a fusion of chimeric peptide or protein, or labeled, e.g., to have an N-terminal FLAG-tag. In a particular embodiment a peptide can be modified by linkage or attachment to a marker protein such as green fluorescent protein as described in U.S. Pat. No. 5,625,048 filed Apr. 29, 1997 and WO 97/26333, published Jul. 24, 1999 (each of which are hereby incorporated by reference herein in their entireties).

In one such embodiment, a chimeric peptide can be prepared, e.g., a glutathione-S-transferase (GST) fusion protein, a maltose-binding (MPB) protein fusion protein, or a poly-histidine-tagged fusion protein, for expression in a eukaryotic cell. Expression of the peptide of the present invention as a fusion protein can facilitate stable expression, or allow for purification based on the properties of the fusion partner. For example, GST binds glutathione conjugated to a solid support matrix, MBP binds to a maltose matrix, and poly-histidine chelates to a Ni-chelation support matrix. The fusion protein can be eluted from the specific matrix with appropriate buffers, or by treating with a protease specific for a cleavage site usually engineered between the peptide and the fusion partner (e.g., GST, MBP, or poly-His). Alternatively the chimeric peptide may contain the green fluorescent protein, and be used to determine the intracellular localization of the peptide in the cell.

Particularly provided are derivatives of the carbohydrate epitope mimic peptides wherein at least one of the attached chemical moieties is a carbohydrate epitope recognizing molecule, for example a neural cell adhesion molecule. More particularly, the neural cell adhesion molecule is selected from the group consisting of L1, N-CAM and myelin-associated glycoprotein. The neural cell adhesion molecule can be selected from the group consisting of laminin, fibronectin, N-cadherin, BSP-2/D2 (mouse N-CAM), 224-1 A6-A1, L1-CAM, NILE (rat L1), Nr-CAM, TAG-1 (axonin-1), Ng-CAM and F3/F11/contactin.

The invention also includes derivatives wherein at least one of the attached chemical moieties is a molecule having multiple sites for peptide attachment and capable of binding at least two of said peptides simultaneously to generate a multimeric peptide structure. This derivative has the effect of increasing the available local concentration of the carbohydrate epitope mimic peptide(s) of the present invention. Alternatively, or in addition, such moieties can function in providing a stable scaffold to retain the peptide in place for activity, thereby reducing or preventing diffusion or degradation. More particularly, such molecule is selected from the group of BSA, ovalbumin, human serum allbumin, polyacrylamide, beads and synthetic fibers (biodegradable and non-biodegradable).

Peptide Monomers, Dimers and Multimers

The carbohydrate epitope mimic peptide of the present invention may be prepared and utilized as monomers, dimers, multimers, heterodimers, heteromultimers, etc. The use of multimers is particularly attractive in view of the activity of carbohydrate epitopes in homophilic and cell-cell interactions. Presentation or administration of the carbohydrate epitope mimic peptide in multimeric form may result in enhanced activity or otherwise increased modulation of the activity mediated by the carbohydrate epitopes, including the activity of carbohydrate epitope recognizing molecules.

Monomers

The carbohydrate epitope mimic peptide monomer could be produced in a variety of ways. The carbohydrate epitope mimic peptide of the present invention can be synthesized using a protein synthesizer and utilizing methods well known in the art and as described hereinabove, incorporating amino acid modifications, analogs, etc. as hereinabove described. In addition, the DNA sequence of the peptide can be inserted into an expression vector such as pSE (Invitrogen) or pcDNA3 (Invitrogen) for production in bacterial or mammalian cell expression systems. Insect or yeast expression systems could also be used. Purification of the peptide could be facilitated by the addition of a tag sequence such as the 6-Histidine tag which binds to Nickel-NTA resins. These tag sequences are often easily removed by the addition of a protease specific sequence following the tag.

Dimers, Multimers

Dimers and multimers of the carbohydrate epitope mimic peptide can be produced using a variety of methods in the art. The DNA sequence of a dimer or multimer could also be inserted into an expression system such as bacteria or mammalian cell systems. This could produce molecules such as Met-FLHTRLFV)ₓ where x=2, 3, 4, . . . etc. It may be necessary to include a short flexible spacer (Gly-Gly-Gly-Gly-Ser)₃ between the peptidomimetic to increase its effectiveness.

Dimers and multimers can also be generated using crosslinking reagents such as Disuccinimidyl suberate (DSS) or Dithoiobis (succinimidyl propionate) (DSP). These reagents are reactive with amino groups and could crosslink the carbohydrate epitope mimic peptide through free amine groups at the arginine residues and the free amine group at the N-terminus.

Dimers and multimers can also be formed using affinity interactions between biotin and avidin, Jun and Fos, and the Fc region of antibodies. The purified arbohydrate epitope mimic peptide can be biotinylated and mixed with factors that are known to form strong protein-protein interactions. The peptidomimetic could be linked to the regions in Jun and Fos responsible for dimer formation using crosslinkers such as those mentioned above or using molecular techniques to create a carbohydrate epitope mimic peptide-Jun/Fos molecule. When the Jun and Fos carbohydrate epitope mimic peptide hybrids are mixed, dimer formation would result. In addition, production of a carbohydrate epitope mimic peptide-Fc hybrid could also be produced. When expressed in mammalian cells, covalent disulfide bonds form through cysteines in the Fc region and dimer formation would result.

Heterodimers, Heteromultimers

Heterodimers and heteromultimers of the carbohydrate epitope mimic peptide could also be produced. This would generate possible multifunctional molecules where parts of the whole molecule are responsible for producing a multitude of effects, such as neuroprotection and neurite outgrowth. The same technologies as those listed above could be used to generate these multifunctional molecules. Molecular techniques could be used to insert the carbohydrate epitope mimic peptide into a protein at the DNA level. This insertion could take place at the N- or C-terminus, or in the middle of the protein molecule. Heterodimers could be formed using carbohydrate epitope mimic peptide/Fc or carbohydrate epitope mimic peptide/June or Fos hybrid molecules. When mixed with other Fc or Jun/Fos containing hybrids dimer formation would result producing heterodimers. Crosslinking reagents could also be used to link the carbohydrate epitope mimic peptide to heterodimers. Lastly, biotinylation of the carbohydrate epitope mimic peptide along with biotinylation of other molecules could be used to create multimers. Mixing of these components with avidin could create large multifunctional complexes, where each of the four biotin binding sites of the avidin molecule is occupied by a different biotinylated molecule.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. This, this term includes double-stranded and single-stranded DNA or RNA molecules.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide iii vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer”as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains about 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand.

Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous. DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

Two DNA sequences are “substantially homologous” when at least about 80% (preferably at least about 90%, and most preferably at least about 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra. Likewise, two polypeptide sequences are “substantially homologous” when at least about 80% (preferably at least about 90%, and most preferably at least about 95%) of the amino acids are either identical or contain conservative changes, as herein defined, over the defined length of the polypeptide sequences. The similar or homologous sequences are identified by alignment using sequence alignment or search programs and methods known to the skilled artisan. Preferably, the similar or homologous sequences are identified by alignment using the GCG pileup program (Genetics Computer Group, Program Manual for the GCG Package, Version 7, Madison Wis.), using the default parameters.

As used herein, the term “about” refers to approximately or close to, usually within (i.e., +/−) 10% of the given value or quantity. For instance, when referring to a length of a peptide as about 20 amino acids, this encompasses between 18 and 22 amino acids. Similarly, an oligonucleotide of about 10 nucleotides encompasses between 9 and 11 nucleotides.

A nucleic acid molecule is “hybridizable” to another nucleic acid molecule, such as a cDNA, genomic DNA, or RNA, when a single-stranded form of the nucleic acid molecule can anneal to the other nucleic acid molecule under the appropriate conditions of temperature and solution ionic strength (see Sambrook et al., 1989, supra). The conditions of temperature and ionic strength determine the “stringency” of the hybridization. For preliminary screening for homologous nucleic acids, low stringency hybridization conditions, corresponding to a Tₘ of 55° C. can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5×or 6×SCC. High stringency hybridization conditions correspond to the highest Tₘ, e.g., 50% formamide, 5×or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., 1989, supra, 9.50-0.51). For hybridization with shorter nucleic acids, i.e., oligonucleotides, the position of mismatches becomes more important, and the length of the oligonucleotide determines its specificity (see Sambrook et al., 1989, supra, 11.7-11.8). Preferably a minimum length for a hybridizable nucleic acid is at least about 10 nucleotides; more preferably at least about 15 nucleotides; most preferably the length is at least about 20 nucleotides.

It should be appreciated that also within the scope of the present invention are DNA sequences capable of encoding the peptides set out in SEQ ID NOS: 1-8, 27-38, 39, 40 or 41, but which are degenerate to the particular exemplary such DNA sequences ie; those degenerate to any of SEQ ID NOS: 9-26 and SEQ ID NOS: 42-50. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA |
| | or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU |
| | or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA |
| | or AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in the DNA sequences of the present invention such that a particular codon is changed to a codon which codes for a different amino acid. Such a mutation is generally made by making the fewest nucleotide changes possible. Additionally, alterations or mutations can be made directly in the amino acid sequence of the peptide(s) of the present invention. This is particularly straightforward in that the particular exemplified peptides and active fragments thereof are of a size which makes them readily synthesized, using methods as previously described and well known in the art. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping), thereby generating a non-conserved variant, or in a conservative manner (i.e., by changing the codon from an amino acid belonging to a grouping of amino acids having a particular size or characteristic to an amino acid belonging to the same grouping), thereby generating a non-conserved variant. Such a conservative change generally leads to less change in the structure and function of the resulting protein. A non-conservative change is more likely to alter the structure, activity or function of the resulting protein. The present invention should be considered to include such variants containing conservative changes or non-conservative changes which do not significantly alter the activity or binding or epitope mimicking characteristics of the resulting peptide.

The following is one example of various groupings of amino acids:

Amino Acids With Nonpolar R Groups

Alanine

Valine

Leucine

Isoleucine

Proline

Phenylalanine

Tryptophan

Methionine

Amino Acids With Uncharged Polar R Groups

Glycine

Serine

Threonine

Cysteine

Tyrosine

Asparagine

Glutamine

Amino Acids With Charged Polar R Groups (Negatively Charged at ph 6.0)

Aspartic acid

Glutamic acid

Basic Amino Acids (Positively Charged at pH 6.0)

Lysine

Arginine

Histidine (at pH 6.0)

Another Grouping may be Those Amino Acids With Phenyl Groups:

Phenylalanine

Tryptophan

Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine | 155 |
| (at pH 6.0) | |
| Phenylalanine | 165 |
| Arginine | 174 |
| Tyrosine | 181 |
| Tryptophan | 204 |

Particularly Preferred Substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Most particularly preferred are substitutions within the following groupings (Altschul, S. F. et al., Nucleic Acids Res 25(17), 3389-3402 (1997); Henikoff, S. and Henikoff, J. G. Proc. Natl. Acad. Sci. 89, 10915-10919 (1992)), each group consisting of amino acids which can be interchanged or substituted in generating conservative amino acid changes or conserved variants:

Valine (V), Isoleucine (I), Leucine (L) and Methionine (M);

Serine (S), Alanine (A) and Threonine (T);

Aspartic Acid (D) and Glutarnic Acid (E);

Arginine (R), Glutamine (O) and Lysine (K);

Tyrosine (Y), Phenylalanine (F), Histidine (H), Tryptophan (W) and Asparagine (N).

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors. Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “heterologous nucleotide sequence” as used herein is a nucleotide sequence that is added to a nucleotide sequence of the present invention by recombinant methods to form a nucleic acid which is not naturally formed in nature. Such nucleic acids can encode chimeric and/or fusion proteins. Thus the heterologous nucleotide sequence can encode peptides and/or proteins which contain regulatory and/or structural properties. In another such embodiment the heterologous nucleotide can encode a protein or peptide that functions as a means of detecting the peptide encoded by the nucleotide sequence of the present invention after the recombinant nucleic acid is expressed. In still anther such embodiment the heterologous nucleotide can function as a means of detecting a nucleotide sequence of the present invention. A heterologous nucleotide sequence can comprise non-coding sequences including restrictions sites, regulatory sites, promoters and the like.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein. Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

As used herein, “pg” means picogram, “ng” means nanogram, “ug” or “μg” mean microgram, “mg” means milligram, “ul” or “μl” mean microliter, “ml” means milliliter, “l” means liter.

In its primary aspect, the present invention concerns the identification of carbohydrate epitope mimic compound(s), particularly peptide(s). Such compounds or peptides particularly mimic the carbohydrate epitope GlcAβ1→3Galβ1→4GlcNAc or sulfate -3GlcAβ1→3Galβ1→4GlcNAc. In a further aspect, the compounds or peptides, are capable of mimicking the L2/HNK1 carbohydrate epitope.

In a particular embodiment, the present invention relates peptides comprising the amino acid sequence set out in any of SEQ ID NOS: 1-8, 27-38, 39, 40 and 41. Particularly preferred are peptides comprising the amino acid F L H T R L F V S D W Y H T (SEQ ID NO: 7), F L H T R L F V (SEQ ID NO: 8), TRLFR(V/F) (SEQ ID NO: 39), TRLF(R)V (SEQ ID NO: 40) or TRLF (SEQ ID NO: 41).

As stated above, the present invention also relates to a recombinant DNA molecule; or a degenerate variant thereof, which encodes a carbohydrate epitope mimic peptide, variant, analog or active fragment thereof, that possesses an amino acid sequence set forth in any of SEQ ID NOS: 1-8, 27-38, 39, 40 and 41, preferably a nucleic acid molecule, in particular a recombinant DNA molecule. Exemplary nucleic acid sequences are those of SEQ ID NOS: 9-26 and SEQ ID NOS: 42-50. Sequences complementary to or degenerate to the DNA sequences of any of SEQ ID NOS: 9-26 and SEQ ID NOS: 42-50 are readily contemplated.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence, or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

It is further intended that carbohydrate epitope mimic peptide variants, analogs and active fragments may be prepared from nucleotide sequences of the peptide derived within the scope of the present invention. Active fragments, may be produced, for example, by proteolytic (e.g., pepsin) digestion of the peptide material, or by direct or chemical synthesis of parts or fragments of the described peptide sequence(s). Variants such as muteins, can be produced by standard site-directed mutagenesis of peptide coding sequences. Analogs exhibiting “carbohydrate epitope mimic activity” such as small molecules or peptides incorporating non-peptide chemical components or unnatural or non-classical amino acids, whether functioning as promoters or inhibitors, may be identified by known in vivo and/or in vitro assays including the assays and methods as described and demonstrated herein.

As mentioned above, a DNA sequence encoding the carbohydrate epitope mimic peptide(s) can be prepared synthetically rather than cloned. The DNA sequence can be designed with the appropriate codons for the carbohydrate epitope mimic peptide amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:,1299(1984); Jay et al., J. Biol. Chem., 259:6311 (1984).

Synthetic DNA sequences allow convenient construction of genes which will express carbohydrate epitope mimic peptide variants or “muteins”. Alternatively, DNA encoding such variants or muteins can be made by site-directed mutagenesis of nucleotide sequences capable of encoding the carbohydrate epitope mimic peptide(s), and muteins can be made directly using conventional polypeptide synthesis.

The present invention extends to the preparation of antisense oligonucleotides and ribozymes that may be used to interfere with the expression of the carbohydrate epitope mimic peptide(s) at the translational level. This approach utilizes antisense nucleic acid and ribozymes to block translation of a specific mRNA, either by masking that mRNA with an antisense nucleic acid or cleaving it with a ribozyme. This might be particularly applicable in interfering with the expression of a carbohydrate epitope mimic peptide from an expression vector.

Antisense nucleic acids are DNA or RNA molecules that are complementary to at least a portion of a specific mRNA molecule. (See Weintraub, 1990; Marcus-Sekura, 1988.) In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into carbohydrate epitope mimic peptide(s)-producing cells. Antisense methods have been used to inhibit the expression of many genes in vitro (Marcus-Sekura, 1988; Hambor et al., 1988).

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, 1988.). Because they are sequence-specific, only mRNAs with particular sequences are inactivated.

Investigators have identified two types of ribozymes, Tetrahymena-type and “hammerhead”-type. (Hasselhoff and Gerlach, 1988) Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The DNA sequences described herein may thus be used to prepare antisense molecules against, and ribozymes that cleave mRNAs for carbohydrate epitope mimic peptide(s) and their ligands.

The possibilities both diagnostic and therapeutic that are raised by thee existence of the carbohydrate epitope mimic peptide(s) derive from the fact that the carbohydrate epitopes appear to participate in direct and causal carbohydrate-protein and protein-protein interaction between the carbohydrate epitope containing molecules and carbohydrate epitope recognizing molecules. In particular, as described earlier, various aspects of cell-cell adhesion and cell-cell interactions involved in cell signaling, cell migration, cell recognition and cell activation are mediated via recognition of or binding to carbohydrate epitopes, particularly the L2/HNK-1 carbohydrate epitope.

Therapeutic Applications

As suggested earlier and elaborated further on herein, the present invention contemplates pharmaceutical intervention in the cascade of reactions in which the carbohydrate epitope, particularly the L2/HNK-1 carbohydrate epitope, is implicated, to modulate the activity initiated by carbohydrate epitope containing molecules and carbohydrate epitope recognizing molecules.

Thus, in instances where insufficient binding or interaction is taking place between and among carbohydrate epitopes, carbohydrate epitope containing molecules and carbohydrate epitope recognizing molecules, this could be remedied by the introduction of the carbohydrate epitope mimic peptide(s) of the present invention, variants, analogs, active fragments and the like. Correspondingly, in instances where it is desired to reduce or inhibit the activity initiated by carbohydrate epitope containing molecules and carbohydrate epitope recognizing molecules, carbohydrate epitope mimic peptide(s) or inhibitors or antagonists thereof could be introduced to block the interaction of carbohydrate epitope containing molecules and carbohydrate epitope recognizing molecules.

Carbohydrate epitopes can exert activating or inhibiting activities by and among carbohydrate epitopes, carbohydrate epitope containing molecules and carbohydrate epitope recognizing molecules. In as much as these activities are mediated by carbohydrate-protein, carbohydrate-carbohydrate or protein-protein interactions, including homophilic interactions, the amount or effective local concentration or degree of cell surface expression of a carbohydrate epitope can influence its activity as activating or inhibitory. Carbohydrate epitopes which are stimulatory can actually become inhibitory at high concentrations. This phenomenon would be expected to be similarly seen for the carbohydrate epitope mimic peptides of the present invention.

The various therapeutic applications of the carbohydrate epitope mimic peptides of the present invention derive from the various aspects of cell-cell adhesion and cell-cell interactions involved in cell signaling, cell migration, cell recognition and cell activation which are mediated via recognition of or binding to carbohydrate epitopes, particularly the L2/HNK-1 carbohydrate epitope. Certain of these particular activities are particularly exemplified in the Examples provided herein. Additional therapeutic applications and uses, particulary of the L2/HNK-1 carbohydrate epitope, will be apparent to the skilled artisan by virtue of the recognized roles of the L2/HNK1 epitope in physiological processes, recognition phenomena and cell-cell interactions, including those outlined and specifically contemplated herein.

Thus, in view of the recognized and previously described role of HNK-1 expressing cells, natural killer cells, in the surveillance of tumors and virus-infected cells, the carbohydrate epitope mimic peptides can be utilized in enhancing, activating or otherwise modulating the surveillance and clearance of tumors and virus-infected cells. The carbohydrate epitope mimic peptides of the present invention may also have use in the protection of cells, particularly neural cells from chemotherapeutic agents. In experiments not specifically detailed in the Examples herein, the inventors treated embryonic neural cell cultures with a combination of the neural cell adhesion molecule L1 and chemotherapeutic agents, including cisplatin and vincristin. The cell cytopathic effects of the chemotherapeutic agents were reduced in te presence of L1. L1expresses the HNK-1 epitope and a good portion of L1 homophilic binding is HNK-1 mediated. Thus, it is contemplated that the L2/HNK-1 epitope mimic peptide of the present invention can be utilized in the protection of cells, particularly neural cells from chemotherapeutic agents. Untoward cellular cytotoxic effects and problematic symptoms associated therewith are a recognized and limiting side effect of chemotherapy, inherently limiting the dose of the agents which can be administered.

In one particular example of the use of the carbohydrate epitope mimic peptides in modulating viral infection, the carbohydrate epitope mimic peptides can be utilized in enhancing, activating or otherwise modulating the surveillance and clearance of HIV virus or HIV virus-infected cells. In view of HIV's ability to infect the immune system and nervous system, and the existence of L2/HNK-1 epitope containing and recognizing molecules in both of these systems, the carbohydrate epitope mimic peptides of the present invention can be utilized in the prevention, amelioration or blocking of HIV infection, both in the immune system, particularly in lymphocytes, and in the nervous system and nervous system cells. As shown in the Examples, the CD4 protein contains a consensus HNK-1 epitope binding sequence and binds L2/HNK-1carbohydrate. Having now recognized an L2/HNK- epitope binding sequence, the skilled artisan can readily identify and/or isolate other L2/HNK-1 interacting molecules containing a homologous or otherwise related L2/HNK-1 epitope binding sequence.

In addition, to the extent that viral infection, viral pathologies or virus-induced cellular alterations are caused by or otherwise related to carbohydrate epitope-mediated interactions, the infections, pathologies or alterations can be inhibited, reduced or prevented by administration or expression of the carbohydrate epitope mimic peptides. For instance, van den Berg and colleagues investigated the binding of the gp120 glycoprotein of HIV to neural glycolipids and glycoproteins by ELISA. The gp120 protein bound to sulfatide (GalS), a sulfated glycolipid autoantigen implicated in sensory neuritis, and to the myelin associated glycoprotein (MAG), an autoantigen in demyelinating neuropathy (van den Berg LH et al (1992) J Neurosc Res 33(4):513-518). Binding of gp120 to MAG was inhibited by the HNK-1 antibody, which recognizes a sulfated glucuronic acid epitope, suggesting that the interaction involves carbohydrate determinants. This is particularly exemplified in the Examples herein, wherein the neuropathy and inflammatory response generated by the HIV envelope glycoprotein gp120 is blocked or reduced in the presence of the L2/HNK-1 epitope mimic peptide of the present invention. The present invention also demonstrates that the cellular effects of gp120 on mature oligodendrocytes is blocked by the L2/HNK-1 epitope mimic peptide of the present invention.

In addition, the Examples provided herein demonstrate that gp120 induced inflammation and peripheral neuropathy is blocked by preincubation with the L2/HNK-1 epitope mimic peptide of the present invention. The peptides of the present invention may therefore be utilized in treatment and prevention of neuropathies associated with viral or immune-mediated disease or resulting form injury to the nervous system, for instance spinal cord injury, head injury or trauma. Patients with MS and PNS neuropathies have been shown to have IgM and/or IgG against peripheral myelin lipids, for instance. Anti-sulfoglucuronyl paragloboside IgM antibodies have also been identified in ALS patients (Ben Younes-Chennoufi A et al (1995)J Neuoimmunol 57(1-2)111-115).

It has also been shown that human cytomegalovirus (HCMV) binds to sulfated glucuronyl glycosphingolipids (SGGLs), particularly to (3GalB1-4GlcNAc1-)2 containing glycolipids and that HNK-1 antibody partially inhibited plaque formation by HCMV (Ogawa-Goto, K et al (1998)J Gen Virology 79:2533-2541). Thus, inhibition or prophylaxis against viral infections, particularly wherein the surface virus proteins interact or otherwise associate with host cells via L2/HNK-1 epitope interactions is contemplated by this invention.

In particular, an L2/HNK1 carbohydrate epitope mimic peptide may be administered to activate or otherwise modulate the activity of L2/HNK-1 recognizing molecules, as in the potentiation or inhibition of neural cell adhesion molecules in CNS or PNS therapy. For instance, it is postulated that the L2/HNK1 carbohydrate epitope mimic peptides may inhibit the inhibitory effects of extracellular matrix molecules such as chondroitin sulfate proteoglycan (CSPG), NG2, Neurocan, Tenascin-C, Tenascin-R etc. which are inhibitory for neurite outgrowth.

Therefore, the present invention includes therapeutic methods for modulating, activating or inhibiting L2/HNK-1 epitope containing or recognizing molecules, particularly neural cell adhesion molecules. Such methods include methods for promoting neural growth and/or remyelination and/or neuroprotection in vivo in the central nervous system of a mammal comprising administering to said mammal a neural growth and/or remyelination and/or neuroprotection promoting amount of the carbohydrate epitope mimic peptide(s) of the present invention, which peptide is capable of overcoming inhibitory molecular cues found on glial cells and myelin and promoting said neural growth, and derivatives, variants, analogs or active fragments thereof, antagonists thereof, antibodies thereto, and secreting or expressing cells thereof Such methods can further incorporate a neural growth and/or remyelination and/or neuroprotection promoting amount of a neural cell adhesion molecule, including a molecule selected from the group of L1, N-CAM, myelin-associated glycoprotein, laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

Method for enhancing memory are also contemplated, comprising administering to the brain of a mammal in need of such enhancement, an amount of the carbohydrate epitope mimic peptide(s) of the present invention, variants, analogs or active fragments thereof effective to enhance the memory of the mammal, partticularly for inhibiting the onset or progression, or treating the presence or consequences of Alzheimers disease or dementia in a mammal.

Similarly, methods for increasing synaptic efficacy, particularly as demonstrated by the stabilization of long term potentiation, are contemplated. Further therapeutic methods include promoting neuroprotection and/or neuronal survival in a mammal, particularly for inhibiting the development or onset, or treating the presence in a mammal of a condition selected from the group consisting of apoptosis, necrosis, Alzheimers disease, dementia, Parkinsons disease, multiple sclerosis, acute spinal cord injury, chronic spinal cord injury, any of the foregoing where neurodegeneration occurs or may occur, and combinations thereof Also, methods are contemplated for inhibiting axonal cell death and enhancing myelination and remyelination in the central nervous system or peripheral nervous system.

Methods are contemplated for preventing, ameliorating or blocking viral infection of a mammal comprising administering to said mammal an effective amount of the carbohydrate epitope mimic peptide, variants thereof, analogs thereof, active fragments thereof or derivatives thereof In a particular embodiment, the viral infection is the result of the human immunodeficiency virus.

Any of such therapeutic methods can utilize any of or any combination of the carbohydrate epitope mimic peptide(s), its derivatives, variants, analogs, active fragments, nucleic acids or DNA molecules capable of encoding such peptides, or vectors or host cells capable of expressing or otherwise presenting such peptides.

As discussed earlier, the carbohydrate epitope mimic peptide(s) or other agents exhibiting either mimicry or antagonism to the carbohydrate epitope mimic peptide(s) or control over their production, may be prepared in pharmaceutical compositions, with a suitable carrier and at a strength effective for administration by various means to a patient experiencing an adverse medical condition associated with specific carbohydrate epitope containing molecules and/or carbohydrate epitope recognizing molecules for the treatment thereof A variety of administrative techniques may be utilized, among them parenteral techniques such as subcutaneous, intravenous and intraperitoneal injections, catheterizations and the like. Average quantities of the carbohydrate epitope mimic peptide(s) or their subunits may vary and in particular should be based upon the recommendations and prescription of a qualified physician or veterinarian.

More specifically, the therapeutic method of the present invention could include the method for the treatment of various pathologies or other cellular dysfunctions and derangements by the administration of pharmaceutical compositions that comprise the carbohydrate epitope mimic peptide(s), derivatives, variants, analogs or active fragments thereof, effective inhibitors or enhancers of activation of the carbohydrate epitope mimic peptide(s), or other equally effective drugs developed for instance by a drug screening assay prepared and used in accordance with the present invention. For example, the carbohydrate epitope mimic peptide(s) of the present invention, variants, analogs or active fragments thereof, as particularly represented by any of SEQ ID NOS: 1-8, 39, 40 and 41 and SEQ ID NOS: 27-38 may be administered to inhibit or potentiate activity of L2/HNK-1 carbohydrate epitope containing molecules or of L2/HNK-1 carbohydrate epitope recognizing molecules, as in the potentiation of neural cell adhesion molecules in CNS or PNS therapy. In particular, the carbohydrate epitope mimic peptide(s) whose sequences are presented in SEQ ID NOS: 1-8, 39, 40 and 41 and SEQ ID NOS: 27-38 herein, variants, analogs, derivatives, agonists, antagonists, or active fragments thereof, could be prepared in pharmaceutical formulations for administration in instances wherein therapy to activate, inhibit or otherwise modulate L2/HNK-1 carbohydrate-recognizing molecules is appropriate, such as to promote neural growth in CNS or PNS therapy. The specificity of the carbohydrate epitope mimic peptide(s) hereof would make it possible to better manage the untoward effects of current CNS or PNS therapy, and would thereby make it possible to apply the carbohydrate epitope mimic peptide(s) as a general neural growth or neuroprotection promoting agent.

Accordingly, present invention provides the carbohydrate epitope mimic peptide(s), variants, analogs, derivatives or active fragments thereof, in purified form, that exhibits certain characteristics and activities associated with the L2/HNK-1 carbohydrate epitope or L2/HNK-1 carbohydrate epitope containing molecules for the promotion or modulation of the activity of L2/HNK-1 carbohydrate epitope recognizing molecules.

The present invention further contemplates therapeutic compositions useful in practicing the therapeutic methods of this invention. A subject therapeutic composition includes, in admixture, a pharmaceutically acceptable excipient (carrier) and one or more of a carbohydrate epitope mimic peptide, variant, analog or active fragment thereof, as described herein as an active ingredient. In a preferred embodiment, the composition comprises the peptide(s) as set out in any of SEQ ID NOS: 1-8, 27-38, 39, 40 and 41. In a further embodiment, the composition further comprises a carbohydrate epitope recognizing molecule or a carbohydrate epitope containing molecule, particularly a neural cell adhesion molecule. Particular examples of neural cell adhesion molecules for use in these compositions include L1, N-CAM, myelin-associated glycoprotein, laminin, fibronectin, N-cadherin, BSP-2/D2 (mouse N-CAM), 224-1A6-A1, NILE (rat L1), Nr-CAM, TAG-1 (axonin-1), Ng-CAM and F3/F11/contactin.

Also contemplated and provided are pharmaceutical compositions for preventing, ameliorating or blocking viral infection comprising a therapeutically effective Amount of the carbohydrate epitope mimic peptide or variants, analogs, derivatives or active fragments thereof and a pharmaceutically acceptable carrier.

The preparation of therapeutic compositions which contain peptides, variants, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof In addition, if desired, the composition can contain minor amounts of auxiliary substances such as wetting or emulsifying agents, pH buffering agents which enhance the effectiveness of the active ingredient.

A peptide, variant, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic peptide-, variant-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's neural or immune system to utilize the active ingredient, and degree of activation or modulation of carbohydrate epitope mimic peptide binding capacity desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and further dosing are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood or similarly appropriate concentrations in the CNS are contemplated.

The therapeutic compositions may further include an effective amount of the carbohydrate epitope mimic peptide(s), variant, analog, active fragment or antagonist thereof, and one or more of the following active ingredients: a neural cell adhesion molecule, a growth factor, a synthetic carbohydrate, an antibiotic, a steroid. Exemplary formulations are given below:

Formulations

| Ingredient | mg/ml | |
|---|---|---|
| Intravenous Formulation I | | |
| cefotaxime | 250.0 | |
| carbohydrate epitope mimic peptide | 10.0 | |
| dextrose USP | 45.0 | |
| sodium bisulfite USP | 3.2 | |
| edetate disodium USP | 0.1 | |
| water for injection q.s.a.d. | 1.0 | ml |
| Intravenous Formulation II | | |
| ampicillin | 250.0 | |
| carbohydrate epitope mimic peptide | 10.0 | |
| sodium bisulfite USP | 3.2 | |
| disodium edetate USP | 0.1 | |
| water for injection q.s.a.d. | 1.0 | ml |
| Intravenous Formulation III | | |
| gentamicin (charged as sulfate) | 40.0 | |
| carbohydrate epitope mimic peptide | 10.0 | |
| sodium bisulfite USP | 3.2 | |
| disodium edetate USP | 0.1 | |
| water for injection q.s.a.d. | 1.0 | ml |
| Intravenous Formulation IV | | |
| carbohydrate epitope mimic peptide | 10.0 | |
| dextrose USP | 45.0 | |
| sodium bisulfite USP | 3.2 | |
| edetate disodium USP | 0.1 | |
| water for injection q.s.a.d. | 1.0 | ml |
| Intravenous Formulation V | | |
| carbohydrate epitope mimic | 5.0 | |
| peptide antagonist | | |
| sodium bisulfite USP | 3.2 | |
| disodium edetate USP | 0.1 | |
| water for injection q.s.a.d. | 1.0 | ml |

According to the invention, the component or components of a therapeutic composition of the invention may be introduced parenterally, transmucosally, e.g., orally, nasally, pulmonarilly, or rectally, intrathecally or transdermally. Preferably, administration is parenteral, e.g., via intravenous injection, and also including, but is not limited to, intra-arteriole, intramuscular, intradermal, subcutaneous, intraperitoneal, intraventricular, and intracranial administration. Oral or pulmonary delivery may be preferred to activate mucosal immunity; since pneumococci generally colonize the nasopharyngeal and pulmonary mucosa, mucosal immunity may be a particularly effective preventive treatment. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In yet another embodiment, the therapeutic compound can be delivered in a controlled release system. For example, the polypeptide may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen Ball (eds.). Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Preferably, a controlled release device is introduced into a subject in proximity of the site of inappropriate immune activation or a tumor. Other controlled release systems are discussed in the review by Langer (Science 249:1527-1533 (1990)).

Also contemplated herein is pulmonary delivery of the peptide of the present invention which acts as carbohydrate epitope mimic peptide (or derivatives thereof). The carbohydrate epitope mimic peptide (or derivative) is delivered to the lungs of a mammal, where it can interfere with bacterial, i.e., streptococcal, and preferably pneumococcal binding to host cells. Other reports of preparation of proteins for pulmonary delivery are found in the art [Adjei et al.(1990) Pharmaceutical Research, 7:565-569; Adjei et al. (1990) International Journal of Pharmaceutics, 63:135-144 (leuprolide acetate); Braquet et al (1989), Journal of Cardiovascular Pharmacology, 13(suppl. 5):143-146 (endothelin-1); Hubbard et al. (1989) Annals of Internal Medicine, Vol. III, pp. 206-212 (α1-antitrypsin); Smith et al.(1989) J. Clin. Invest. 84:1145-1146 (α-1-proteinase); Oswein et al., “Aerosolization of Proteins”, Proceedings of Symposium on Respiratory Drug Delivery II, Keystone, Colo., March, (1990) (recombinant human growth hormone); Debs et al.(1988) J. Immunol. 140:3482-3488 (interferon-γ and tumor necrosis factor alpha); Platz et al., U.S. Pat. No. 5,284,656 (granulocyte colony stimulating factor)]. A method and composition for pulmonary delivery of drugs is described in U.S. Pat. No. 5,451,569, issued Sep. 19, 1995 to Wong et al.

All such devices require the use of formulations suitable for the dispensing of carbohydrate epitope mimic peptide inhibitory agent (or derivative). Typically, each formulation is specific to the type of device employed and may involve the use of an appropriate propellant material, in addition to the usual diluents, adjuvant and/or carriers useful in therapy. Also, the use of liposomes, microcapsules or microspheres, inclusion complexes, or other types of carriers is contemplated. Chemically modified carbohydrate epitope mimic peptide inhibitory agent may also be prepared in different formulations depending on the type of chemical modification or the type of device employed.

Formulations suitable for use with a nebulizer, either jet or ultrasonic, will typically comprise epitope mimic peptide inhibitory agent (or derivative) dissolved in water at a concentration of about 0.1 to 25 mg of biologically active carbohydrate epitope mimic peptide per ml of solution. The formulation may also include a buffer and a simple sugar (e.g., for carbohydrate epitope mimic peptide stabilization and regulation of osmotic pressure). The nebulizer formulation may also contain a surfactant, to reduce or prevent surface induced aggregation of the carbohydrate epitope mimic peptide caused by atomization of the solution in forming the aerosol.

Formulations for use with a metered-dose inhaler device will generally comprise a finely divided powder containing the carbohydrate epitope mimic peptide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1, 1,2-tetrafluoroethane, or combinations thereof Suitable surfactants include sorbitan trioleate and soya lecithin. Oleic acid may also be useful as a surfactant.

The liquid aerosol formulations contain carbohydrate epitope mimic peptide and a dispersing agent in a physiologically acceptable diluent. The dry powder aerosol formulations of the present invention consist of a finely divided solid form of carbohydrate epitope mimic peptide and a dispersing agent. With either the liquid or dry powder aerosol formulation, the formulation must be aerosolized. That is, it must be broken down into liquid or solid particles in order to ensure that the aerosolized dose actually reaches the mucous membranes of the nasal passages or the lung. The term “aerosol particle” is used herein to describe the liquid or solid particle suitable for nasal or pulmonary administration, i.e., that will reach the mucous membranes. Other considerations, such as construction of the delivery device, additional components in the formulation, and particle characteristics are important. These aspects of pulmonary administration of a drug are well known in the art, and manipulation of formulations, aerosolization means and construction of a delivery device require at most routine experimentation by one of ordinary skill in the art. In a particular embodiment, the mass median dynamic diameter will be 5 micrometers or less in order to ensure that the drug particles reach the lung alveoli [Wearley, L. L. (1991) Crit. Rev. in Ther. Drug Carrier Systems 8:333].

Systems of aerosol delivery, such as the pressurized metered dose inhaler and the dry powder inhaler are disclosed in Newman, S. P., Aerosols and the Lung, Clarke, S. W. and Davia, D. editors, pp. 197-22 and can be used in connection with the present invention.

In a further embodiment, as discussed in detail infra, an aerosol formulation of the present invention can include other therapeutically or pharmacologically active ingredients in addition to carbohydrate epitope mimic peptide, such as but not limited to an antibiotic, a steroid, a non-steroidal anti-inflammatory drug, etc.

Liquid Aerosol Formulations. The present invention provides aerosol formulations and dosage forms for use in treating subjects suffering from bacterial, e.g., streptococcal, in particularly pneumococcal, infection. In general such dosage forms contain carbohydrate epitope mimic peptide in a pharmaceutically acceptable diluent. Pharmaceutically acceptable diluents include but are not limited to sterile water, saline, buffered saline, dextrose solution, and the like. In a specific embodiment, a diluent that may be used in the present invention or the pharmaceutical formulation of the present invention is phosphate buffered saline, or a buffered saline solution generally between the pH 7.0-8.0 range, or water.

The liquid aerosol formulation of the present invention may include, as optional ingredients, pharmaceutically acceptable carriers, diluents, solubilizing or emulsifying agents, surfactants and excipients. The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

The formulations of the present embodiment may also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

The present invention further contemplates liquid aerosol formulations comprising carbohydrate epitope mimic peptide and another therapeutically effective drug, such as an antibiotic, a steroid, a non-steroidal anti-inflammatory drug, etc.

Aerosol Dry Powder Formulations. It is also contemplated that the present aerosol formulation can be prepared as a dry powder formulation comprising a finely divided powder form of carbohydrate epitope mimic peptide and a dispersant.

Formulations for dispensing from a powder inhaler device will comprise a finely divided dry powder containing carbohydrate epitope mimic peptide (or derivative) and may also include a bulking agent, such as lactose, sorbitol, sucrose, or mannitol in amounts which facilitate dispersal of the powder from the device, e.g., 50 to 90% by weight of the formulation. The carbohydrate epitope mimic peptide (or derivative) should most advantageously be prepared in particulate form with an average particle size of less than 10 mm (or microns), most preferably 0.5 to 5 mm, for most effective delivery to the distal lung. In another embodiment, the dry powder formulation can comprise a finely divided dry powder containing carbohydrate epitope mimic peptide, a dispersing agent and also a bulking agent. Bulking agents useful in conjunction with the present formulation include such agents as lactose, sorbitol, sucrose, or mannitol, in amounts that facilitate the dispersal of the powder from the device.

The present invention further contemplates dry powder formulations comprising carbohydrate epitope mimic peptide and another therapeutically effective drug, such as an antibiotic, a steroid, a non-steroidal anti-inflammatory drug, etc.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the component or components (or chemically modified forms thereof) and inert ingredients which allow for protection against the stomach environment, and release of the biologically active material in the intestine.

Also specifically contemplated are oral dosage forms of the above derivatized component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Abducts” In: Enzymes as Drugs, Hocenberg and Roberts, eds., Wiley-Interscience, New York, N.Y., pp. 37-383; Newmark, et al. (1982) J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The peptide therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextran and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the polypeptide (or derivative) are for instance the fatty acids oleic acid, linoleic acid and linolenic acid.

Pulmonary Delivery. Also contemplated herein is pulmonary delivery of the present polypeptide (or derivatives thereof). The polypeptide (or derivative) is delivered to the lungs of a mammal while inhaling and coats the mucosal surface of the alveoli. Other reports of this include Adjei et al. (1990) Pharmaceutical Research 7:565-569; Adjei et al. (1990) International Journal of Pharmaceutics 63:135-144 (leuprolide acetate); Braquet et al. (1989) Journal of Cardiovascular Pharmacology, 13(suppl. 5): 143-146 (endothelin-1); Hubbard et al (1989) Annals of Internal Medicine, Vol. III, pp. 206-212 (α1-antitrypsin); Smith et al. (1989) J. Clin. Invest. 84:1145-1146 (α-1-proteinase); Oswein et al (1990) “Aerosolization of Proteins”, Proceedings of Symposium on Respiratory Drug Delivery II, Keystone, Colo., March, (recombinant human growth hormone); Debs et al. (1988) J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Formulations suitable for use with a nebulizer, either jet or ultrasonic, will typically comprise polypeptide (or derivative) dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the polypeptide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof Suitable surfactants include sorbitan trioleate and soya lecithin. Oleic acid may also be useful as a surfactant.

Formulations for dispensing from a powder inhaler device will comprise a finely divided dry powder containing polypeptide (or derivative) and may also include a bulking agent, such as lactose, sorbitol, sucrose, or mannitol in amounts which facilitate dispersal of the powder from the device, e.g., 50 to 90% by weight of the formulation. The protein (or derivative) should most advantageously be prepared in particulate form with an average particle size of less than 10 mm (or microns), most preferably 0.5 to 5 mm, for most effective delivery to the distal lung.

Nasal Delivery. Nasal or nasopharyngeal delivery of the polypeptide (or derivative) is also contemplated. Nasal delivery allows the passage of the polypeptide directly over the upper respiratory tract mucosal after administering the therapeutic product to the nose, without the necessity for deposition of the product in the lung. Formulations for nasal delivery include those with dextran or cyclodextran.

Diagnostic Applications

Also, antibodies including both polyclonal and monoclonal antibodies, and drugs that modulate the production or activity of the carbohydrate epitope mimic peptide(s) and/or their subunits may possess certain diagnostic applications and may for example, be utilized for the purpose of detecting and/or measuring conditions such as neural damage, remyelination, demyelination, viral infection or the like. For example, the carbohydrate epitope mimic peptide(s) or variants, analogs or active fragments thereof may be used to produce both polyclonal and monoclonal antibodies to themselves in a variety of cellular media, by known techniques such as the hybridoma technique utilizing, for example, fused mouse spleen lymphocytes and myeloma cells. Alternatively, available and previously described antibodies, such as L2-412 and HNK-1 may be utilized. Likewise, small molecules that mimic or antagonize the activity(ies) of the carbohydrate epitope mimic peptide(s) of the invention may be discovered or synthesized, and may be used in diagnostic and/or therapeutic protocols. In addition, known L2/HNK-1 carbohydrate epitope recognizing molecules, such as laminin, selectin, N-CAM, L1, etc., may be utilized.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against carbohydrate epitope mimic peptide(s) can be screened for various properties; i.e., isotope, epitope, affinity, etc. Of particular interest are monoclonal antibodies that neutralize the activity of the carbohydrate epitope mimic peptide(s) or its subunits. Such monoclonals can be readily identified in carbohydrate epitope mimic peptide activity assays. High affinity antibodies are also useful when immunoaffinity purification of native or recombinant carbohydrate epitope mimic peptide(s) is possible.

Preferably, the anti-peptide antibody used in the diagnostic methods of this invention is an affinity purified polyclonal antibody. More preferably, the antibody is a monoclonal antibody (mAb). In addition, it is preferable for the anti-peptide antibody molecules used herein be in the form of Fab, Fab′, F(ab′)₂ or F(v) portions of whole antibody molecules.

As suggested earlier, the diagnostic method of the present invention comprises examining a cellular sample or medium by means of an assay including an effective amount of a carboyhdrate epitope recognizing molecule, such as an anti-peptide antibody, L2-412 antibody, HNK-1 antibody, laminin, selectin, L1, or N-CAM, preferably an affinity-purified polyclonal antibody, and more preferably a mAb. In addition, it is preferable for the anti-carbohydrate epitope or anti-peptide antibody molecules used herein be in the form of Fab, Fab′, F(ab′)₂ or F(v) portions or whole antibody molecules. As previously discussed, patients capable of benefitting from this method include those suffering from cancer, a pre-cancerous lesion, a viral infection or other like pathological derangement. Methods for isolating the peptide and inducing anti-peptide antibodies and for determining and optimizing the ability of anti-carbohydrate epitope antibodies to assist in the examination of the target cells are all well-known in the art.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a carbohydrate epitope mimic peptide or synthetic carbohydrate. Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the present paptide and their ability to inhibit specified binding activity in target cells or to target substrates.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques. Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

The present invention also relates to a variety of diagnostic applications, including methods for detecting the presence of carbohydrate epitope mimic peptides, by reference to their ability to elicit or competitively inhibit the activities which are mediated by the present carbohydrate epitope mimic peptides.

As described in detail above, antibody(ies) to the carbohydrate epitope mimic peptides can be produced and isolated by standard methods including the well known hybridoma techniques. For convenience, the antibody(ies) to the carbohydrate epitope mimic peptides will be referred to herein as Ab₁ and antibody(ies) raised in another species as Ab₂.

The presence of carbohydrate epitope mimic peptide(s) or of carbohydrate epitope recognizing molecules or of carbohydrate epitope containing molecules in cells or in a sample can be ascertained by the usual immunological procedures applicable to such determinations. A number of useful procedures are known. Three such procedures which are especially useful utilize either the carbohydrate epitope mimic peptide(s) labeled with a detectable label, antibody Ab₁ labeled with a detectable label, or antibody Ab₂ labeled with a detectable label. The procedures may be summarized by the following equations wherein the asterisk indicates that the particle is labeled, and “peptide” stands for the carbohydrate epitope mimic peptides:

A. peptide*+Ab₁=peptide*Ab₁

B. peptide+Ab*=peptide Ab₁*

C. peptide+Ab₁+Ab₂*=peptide Ab₁Ab₂*

The procedures and their application are all familiar to those skilled in the art and accordingly may be utilized within the scope of the present invention. The “competitive” procedure, Procedure A, is described in U.S. Pat. Nos. 3,654,090 and 3,850,752. Procedure C, the “sandwich” procedure, is described in U.S. Pat. Nos. RE 31,006 and 4,016,043. Still other procedures are known such as the “double antibody,” or “DASP” procedure.

In each instance, the carbohydrate epitope mimic peptide forms complexes with one or more antibody(ies) or binding partners (e.g., carbohydrate epitope containing or recognizing molecules) and one member of the complex is labeled with a detectable label. The fact that a complex has formed and, if desired, the amount thereof, can be determined by known methods applicable to the detection of labels.

It will be seen from the above, that a characteristic property of Ab₂ is that it will react with Ab₁. This is because Ab₁ raised in one mammalian species has been used in another species as an antigen to raise the antibody Ab₂. For example, Ab₂ may be raised in goats using rabbit antibodies as antigens. Ab₂ therefore would be anti-rabbit antibody raised in goats. For purposes of this description and claims, Ab₁ will be referred to as a primary or anti-carbohydrate epitope antibody, and Ab₂ will be referred to as a secondary or anti-Ab₁ antibody.

The labels most commonly employed for these studies are radioactive elements, enzymes, dyes, chemicals which fluoresce when exposed to ultraviolet light, and others. A number of fluorescent materials are known and can be utilized as labels. Examples of labels include, for example, fluorescein, rhodamine, auramine, Texas Red, AMCA blue and Lucifer Yellow, Green Fluorescent Protein (GFP), horse radish peroxidase (HRP) and beta-galactosidase. A particular detecting material is anti-rabbit antibody prepared in goats and conjugated with fluorescein through an isothiocyanate.

The peptide or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A particular assay system developed and utilized in accordance with the present invention, is known as a receptor assay. In a receptor assay, the material to be assayed is appropriately labeled and then certain cellular test colonies are inoculated with a quantity of both the labeled and unlabeled material after which binding studies are conducted to determine the extent to which the labeled material binds to the cell receptors. In this way, differences in affinity between materials can be ascertained.

Accordingly, a purified quantity of the carbohydrate epitope mimic peptide may be radiolabeled and combined, for example, with antibodies or other inhibitors thereto, after which binding studies would be carried out. Solutions would then be prepared that contain various quantities of labeled and unlabeled uncombined carbohydrate epitope mimic peptide, and cell samples would then be inoculated and thereafter incubated. The resulting cell monolayers are then washed, solubilized and then counted in a gamma counter for a length of time sufficient to yield a standard error of <5%. These data are then subjected to Scatchard analysis after which observations and conclusions regarding material activity can be drawn. While the foregoing is exemplary, it illustrates the manner in which a receptor assay may be performed and utilized, in the instance where the cellular binding ability of the assayed material may serve as a distinguishing characteristic.

An assay useful and contemplated in accordance with the present invention is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784 and PCT International Publication No. WO 88/03168, for which purpose the artisan is referred.

In a further embodiment of this invention, commercial test kits suitable for use by a medical specialist may be prepared to determine the presence or absence of predetermined carbohydrate epitope mimic peptide activity or predetermined carbohydrate epitope recognizing activity capability in suspected target cells or sample. In accordance with the testing techniques discussed above, one class of such kits will contain at least the labeled carbohydrate epitope mimic peptide or its binding partner, for instance an antibody specific thereto or a carbohydrate recognizing molecule (such as laminin), and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence or capability of cells for predetermined carbohydrate epitope mimicking activity, comprising:

(a) a predetermined amount of at least one labeled immunochemically reactive component obtained by the direct or indirect attachment of the carbohydrate epitope mimic peptide or a specific binding partner thereto, to a detectable label;

(b) other reagents; and

(c) directions for use of said kit.

More specifically, the diagnostic test kit may comprise:

(a) a known amount of the carbohydrate epitope mimic peptide as described above (or a binding partner) generally bound to a solid phase to form an immunosorbent, or in the alternative, bound to a suitable tag, or plural such end products, etc. (or their binding partners) one of each;

(b) if necessary, other reagents; and

(c) directions for use of said test kit.

In a further variation, the test kit may be prepared and used for the purposes stated above, which operates according to a predetermined protocol (e.g. “competitive,” “sandwich,” “double antibody,” etc.), and comprises:

(a) a labeled component which has been obtained by coupling the carbohydrate epitope mimic peptide to a detectable label;

(b) one or more additional immunochemical reagents of which at least one reagent is a ligand or an immobilized ligand, which ligand is selected from the group consisting of:

(i) a ligand capable of binding with the labeled component (a);

(ii) a ligand capable of binding with a binding partner of the labeled component (a);

(iii) a ligand capable of binding with at least one of the component(s) to be determined; and

(iv) a ligand capable of binding with at least one of the binding partners of at least one of the component(s) to be determined; and

(c) directions for the performance of a protocol for the detection and/or determination of one or more components of an immunochemical reaction between the carbohydrate epitope mimic peptide and a specific binding partner thereto.

In accordance with the above, an assay system for screening potential drugs effective to modulate the activity of the carbohydrate epitope mimic peptide may be prepared. The carbohydrate epitope mimic peptide may be introduced into a test system, and the prospective drug may also be introduced into the resulting test system, and the system thereafter examined to observe any changes in the carbohydrate epitope mimic peptide activity therein, due either to the addition of the prospective drug alone, or due to the effect of added known quantities of the carbohydrate epitope mimic peptide.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### Isolation of a Peptide Mimic of an HNK-1 Related Carbohydrate Structure

The L2/HNK-1 carbohydrate occurs in biologically active glycoproteins and glycolipids in the immune and nervous systems and has been recognized as an important ligand in various cell-cell and cell-substrate interactions. The carbohydrate may contribute to the preferential reinnervation of motor nerve by regenerating motor axons in vivo. The carbohydrate is recognized by the so-called HNK-1 monoclonal antibody. It is likely that the trisaccharide sulfate -3GlcAβ1→3Galβ1→4GlcNAc represents the minimal structure necessary for HNK-1 recognition, with the sulfate group required for binding to HNK-1 antibody. The monoclonal antibody L2-412, by contrast, binds to both sulfated and non-sulfated forms of the carbohydrate. Screening of phage-displayed random peptide libraries represents a powerful means of identifying peptide ligands for targets of interest. Based on this method, we have isolated a collection of phages expressing peptides that bind to the L2-412 antibody. These peptides share a consensus sequence of 8 amino acids. The selected peptide can compete with the interaction between the L2-412 antibody and glycolipids or glycoproteins carrying the L2/HNK-1 carbohydrate. Phages bearing the selected peptide of interest promote neurite outgrowth from motor neurones in vitro.

The development of a highly complex network such as the nervous system requires the controlled outgrowth of neurites and the formation of the correct synaptic connections. The extension of the neurites depends on the interaction of receptor molecules with the extracellular matrix and with the cell surfaces of surrounding neuronal or non-neuronal cells. There is increasing evidence that carbohydrates, carried by cell surface and extracellular matrix glycoproteins or by glycolipids, are involved in the recognition processes that determine the interaction of neural cells with their environment (Schachner and Martini, 1995). The L2/HNK-1 carbohydrate is expressed on recognition molecules, for instance of the immunoglobulin superfamily and on extracellular matrix glycoproteins and integrins (Schachner and Martini, 1995) It specifically binds to certain isoforms of laminin (Hall, H. et al., Eur. J. Neurosci. 5, 34-42 (1993)).

The L2/HNK-1 carbohydrate epitope is also the target for autoimmune IgM antibodies in demyelinating neuropathies of the peripheral nervous system in humans (for a review, see Steck, 1993), Ilyas, A. A. et al. Proc. Natl. Sci. USA 81, 1225-9 (1984)). It has been recently observed that these antibodies from human patients cause demyelination in chicken, confirming their involvement in damaging nervous tissue (Tatum, 1993).

The finding that the L-2/HNK-1 carbohydrate is specifically expressed by myelinating Schwann cells surrounding motor but not sensory axons of the mouse femoral nerve (Martini, 1992), and the fact that the motor neuron in vitro preferentially grow on substrates derivatized with the L2/HNK-1 carbohydrate underline the importance of this carbohydrate in a recognition process in the nervous system.

Chou and Jungalwala (1986) have described the structure of the major antigenic glycolipid present in human peripheral nerve that contains sulfated glucuronic acid and reacts with HNK-1 antibody. The structure, sugar linkage configuration and position of the sulphate group was characterized as sulfate -3-GlcAβ(1-3) Galβ(1-4) GlcNAcβ(1-3) Galβ(1-4) Glcβ(1-1)-ceramide. More recently, the structure of an HNK-1-reactive carbohydrate epitope of bovine peripheral myelin glycoprotein (PO) has been elucidated (Voshol, 1996). It contains the same terminal trisaccharide as in the glycolipid determined by Chou and Jungalwala suggesting that this structure is sufficient for its immunoreactivity. Thus, the carbohydrate epitope present on various L2/HNK-1 antibody reaction cells and in various L2/HNK-1 epitope containing molecules corresponds in core structure to GlcAβ1→3Galβ→4GlcNAc.

A major obstacle in the investigation of biological functions of complex carbohydrates is the availability of these compounds. They can often be isolated from biological sources only in minute amounts (2.5 mg per kg of cauda equina) and the synthesis of a complicated oligosaccharide structure, such as the HNK-1 epitope, is a complicated and lengthy process (Ogawas et al.).

A possible solution to this problem is to mimic carbohydrates by other compounds that are easier to prepare, e.g., peptides. The most promising way to find such peptides is by use of the random peptide phage display technology. In this approach, peptides or proteins are expressed on the tip of a filamentous phage, as a fusion protein with the phage surface protein pilus (Devlin, 1992), (Cwirla, S. E. et al., Proc. Natl. Acad. Sci. USA, 87, 6378-6882 (1990)). Screening phage-displayed random peptide libraries represents a powerful means of identifying peptides ligands for targets of interest. Phage expressing binding peptides are selected by affinity purification with the target of interest. Peptide ligands identified in the manner frequently interact with natural binding site(s) on the target molecule and often resemble the target's natural ligand(s). Although this system is most often used to identify peptide epitopes, it has also been successfully applied to the carbohydrate binding site of the pectin concanavalin A (Scott, 1992). Peptides that mimic the binding of methyl a-D-mannopyranoside to ConA were identified by screening a phage-displayed random hexa-or decapeptide library (Scott, 1992), (Oldenburg, R. K. et al., Proc. Natl Acad. Sci. 89, 5393-5397 (1992)). Peptides with the consensus sequences YPY were found to bind specifically to the carbohydrate binding site with affinity constants of up to 18 μM. In this work, we have found a peptide which can mimic the L2/HNK-1 carbohydrate epitope in terms of function and interaction with the natural binding partners.

It is expected that a carbohydrate mimic peptide will lead to a better understanding of the biological activity of the L2/HNK-1 carbohydrate. The peptide can be produced in much larger amounts than the natural L2/HNK-1 carbohydrate. It may therefore be tested for example, in applications directed towards promoting regeneration of the peripheral nervous system, particularly of motor axons.

Isolation of Mimic Peptides

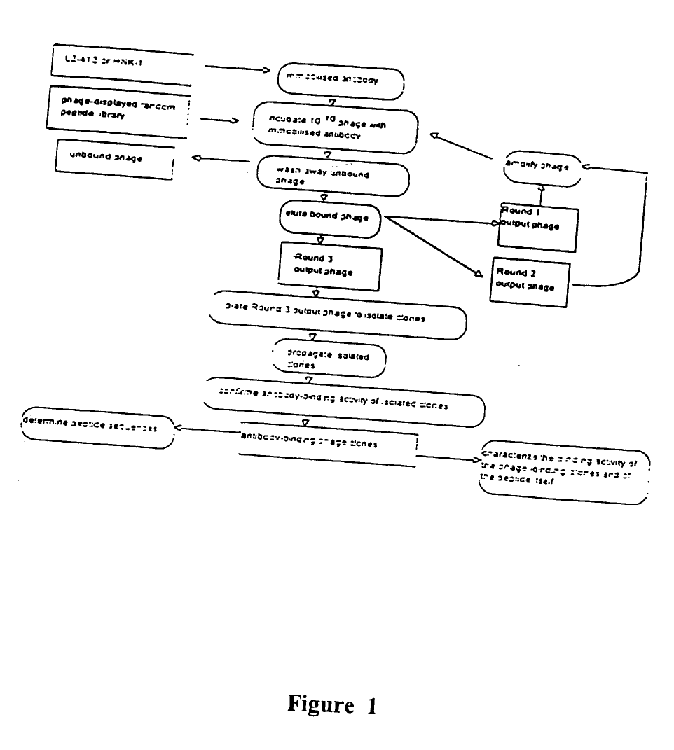

The library, consisting of 2×10⁸ original. clones, was screened in three cycles of panning with the antibody L2-412, elution with pH shift and amplification as shown in FIG. 1. Increasing number of binders were observed in successive rounds of screening (10⁻⁴% to 0.1%) suggesting that selective phages enrichment was occurring. After the third round of panning, 96 clones were tested on plates coated with L2-412 or rat IgG in an ELISA System. Then 20 clones, chosen from those binding to L2-412 but not to rat IgG, were sequenced as described in Materials and Methods. Deduced peptide sequences are shown in Table 2 (respectively as listed in the table clone 15-3 (SEQ ID NO: 27), clone 15-15 (SEQ ID NO: 28), clones 15-94, 91, 97 and 96 (SEQ ID NO: 29), clones 15-cho4, cho6, cho3 and cho1 (SEQ ID NO: 30), clone 15-81 (SEQ ID NO: 31), clones 15-84 and 15-L2 (SEQ ID NO: 32), clone 15-ph1 (SEQ ID NO: 33) and clones 15-5, 13, 14, 16, 23, 24, 31, 32 and 40 (SEQ ID NO: 28). Clone 15(1BR2) (SEQ ID NO: 33) is an unbound control phage.

TABLE 2
Sequences obtained in parallel screenings.

| | | no. of | |
|---|---|---|---|
| | | times | |
| Sequences | clone name | isolated | screening |
| T F T R V V T D V Y R G R L S | 15-3 | 4 | 1 |
| F L H T R L F V S D W Y H T P | 15-15 | 4 | 1 |
| F L H T R L F V S D W Y H T P | 15-94, 91, | 4 | 2 |
| | 97, 96 | | |
| F L H T R L F V S D W Y N T P | 15-cho4, | 4 | 2 |
| | cho6, | | |
| | cho3, cho1 | | |
| F L H T R L L F R I V S Y S G | 15-81 | 1 | 2 |
| F L H T R L L F R N G I I L R | 15-84, 15- | 2 | 2 |
| | L2 | | |
| F L H T R L F V S D G I N S G | 15-ph1 | 1 | 2 |
| F L H T R L F V S D W Y H T P | 15-5, 13, | 8 | 3 |
| | 14, 16, | | |
| | 23, 24, | | |
| | 31, 32, 40 | | |
| S G R G F C C W S N D S A L S | 15 (UBR2) | 1 | 2 |

In the column names “screening”, number “1” corresponds to the first screening that was described in section 3.1. Number “2” corresponds to the second screening (section 3.6) done using the three different elution buffers. The clone names using the “L2” nomenclature means that this clone was isolated using the “rest-L2” elution buffer, the nomenclature “-cho” means these clones were isolated using the SO₅-sugar elution buffer and the “-ph” named clone was isolated with the acidic pH shift. Screening number “3” corresponds to the third parallel screening done under the conditions described in section 3.1 UBR2 (unbound random) is the sequence found on the control phage.

These peptides show a consensus sequence FLHTRLFV (SEQ ID NO:8), and one of these peptides was found 12 times. This indicates that this particular sequence was strongly selected and amplified over the others. The 15-mer peptide FLHTRLFVSDWYHT (15-15) (SEQ ID NO: 7) was then chemically synthesized and its ability to bind to other ligands of the L2/HNK-1 carbohydrate such as laminin was tested. The functional properties of the peptide mimic of the HNK-1 carbohydrate was further studied and its influence on neurite outgrowth of chicken motor neurons evaluated. The complete sequence was initially selected because: 1) it contains the FLHTRLFV (SEQ ID NO: 8) consensus sequence and 2) since it was found 12 times, the rest of the sequence might be of potential importance for stabilizing or presenting correctly the consensus sequence to the mAbL2-412. A randomized form of the 15-15 sequence was also synthesized (Table 3). In both cases the peptides were freshly synthesized and coupled to BSA.

Competition Assay With the Selected Peptide

To demonstrate that the peptide did not bind non-specifically to the surface of the L2-412 antibody molecule outside of the antigen-combining site, we tested the 15-15 peptide in different competition experiments.

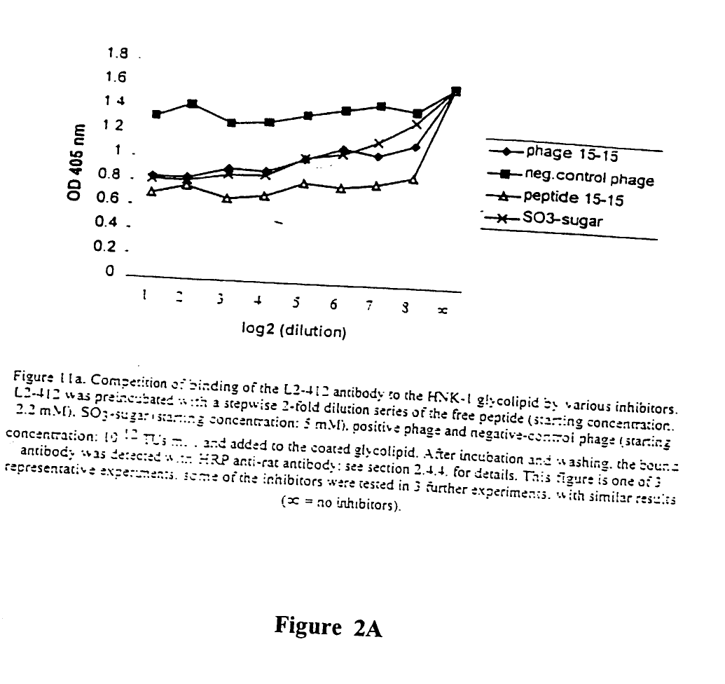

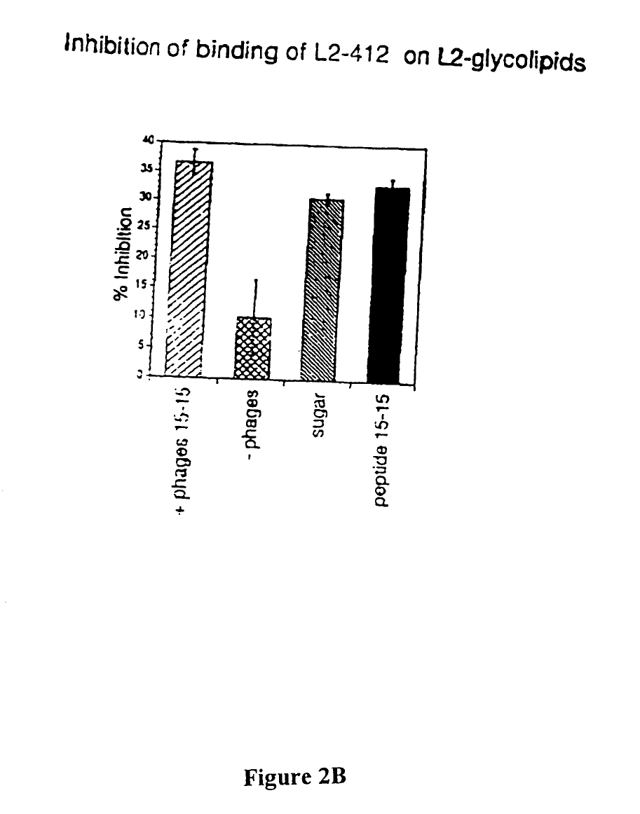

In the first experiment shown in FIG. 2A and B, we compared the effect of the free peptide, the positive phage, negative phage and SO₃-sugar on the binding of L2-412 to immobilized L2-glycolipids. These experiments were done using different amounts of the inhibitors in solutions in the presence of a pre-determined limiting concentration of mAb (see Material and Methods). These inhibition studies show a similar inhibitory effect (30-35%) for the positive phages, the free peptide itself or the synthetic SO₃-sugar. The control, the negative phage randomly chosen among the unbound phages of the first round of selection, did not show this inhibitory effect.

Because a high concentration of free peptide was needed, we assessed whether better competition would be seen with the peptide coupled to BSA. Varying concentrations of the peptide coupled to BSA were pre-incubated with a limiting amount of mAbL2-412 and then incubated together on the immobilized glycolipid. The 15-mer pept/BSA was able to inhibit 30% of the L2-412 binding to the L2-glycolipid. The effect was comparable to that obtained with the free peptide.

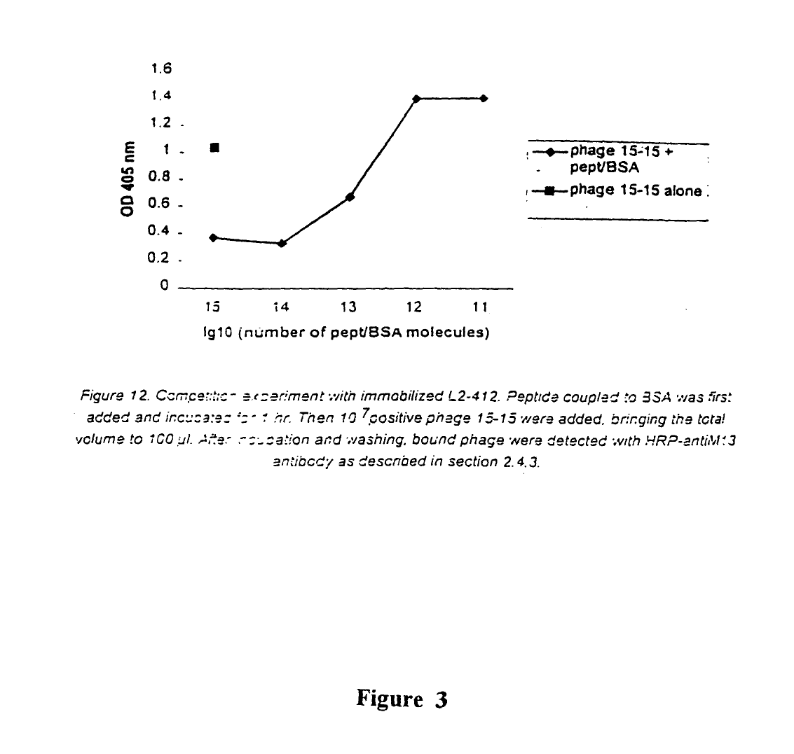

Since we knew from previous experiments that the positive phage bind to L2-412, the 15-15 peptide coupled to BSA was expected to compete with this binding. FIG. 3 shows an experiment in which different concentrations of the peptide coupled to BSA could indeed inhibit the binding of the positive phage to the immobilized L2-412.

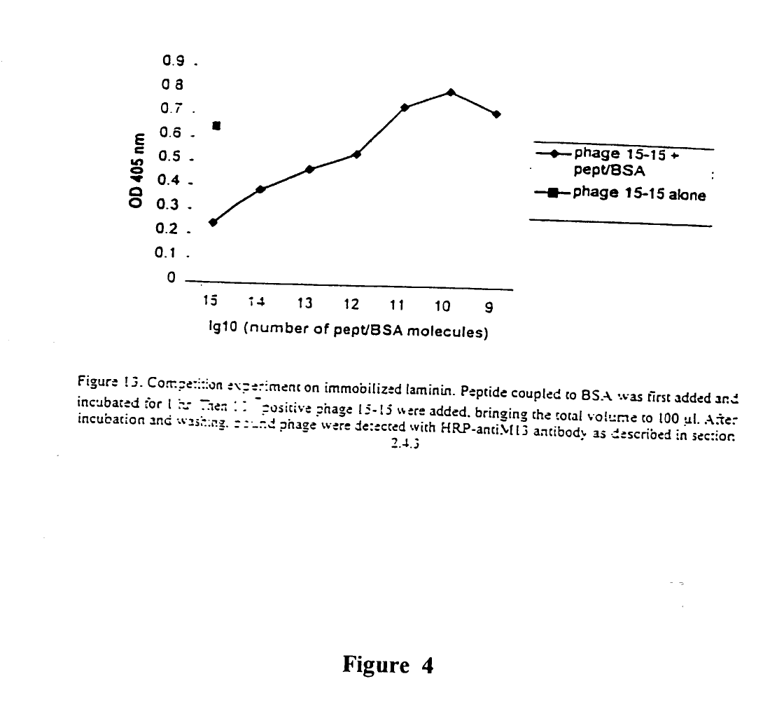

Similarly, FIG. 4 shows that the peptide coupled to BSA was able to compete with the binding of the positive phage to laminin, a binding partner of the L2/HNK-1 carbohydrate. The positive phage was also shown to bind to laminin in a concentration dependent manner (FIG. 5).

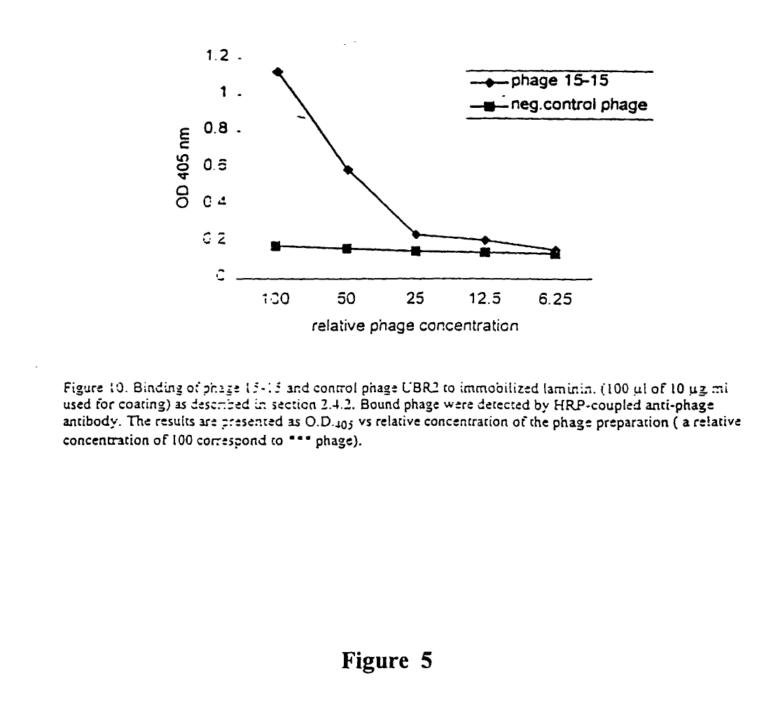

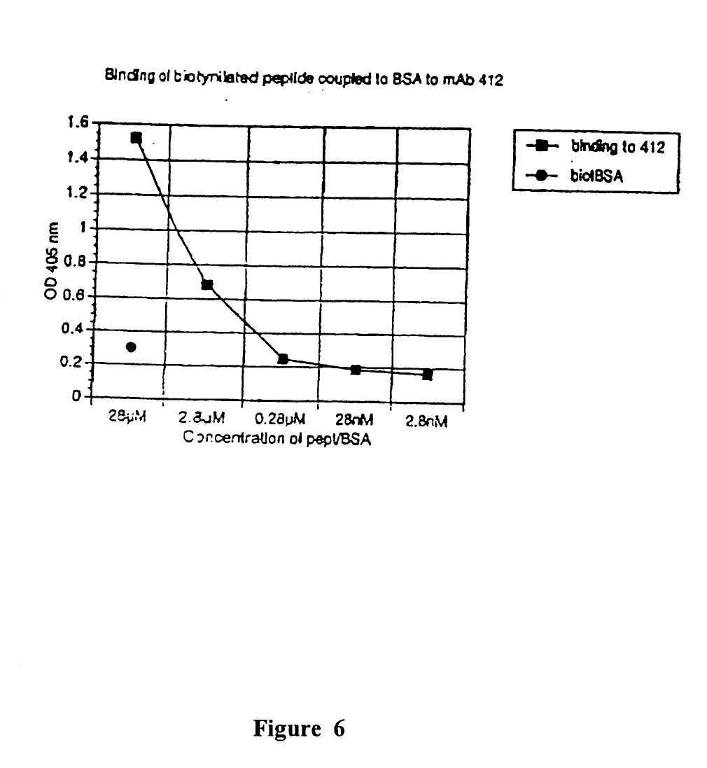

In order to confirm the inhibition experiment, we performed two solid-phase binding studies using biotinylated peptide to show the direct binding of the biotinylated peptide-BSA complex. The first experiment, shown in FIG. 6, demonstrates that the biotinylated pept/BSA binds to the mAbL2-412 in a concentration-dependent manner. The biotinylated BSA used as a control did not show any binding.

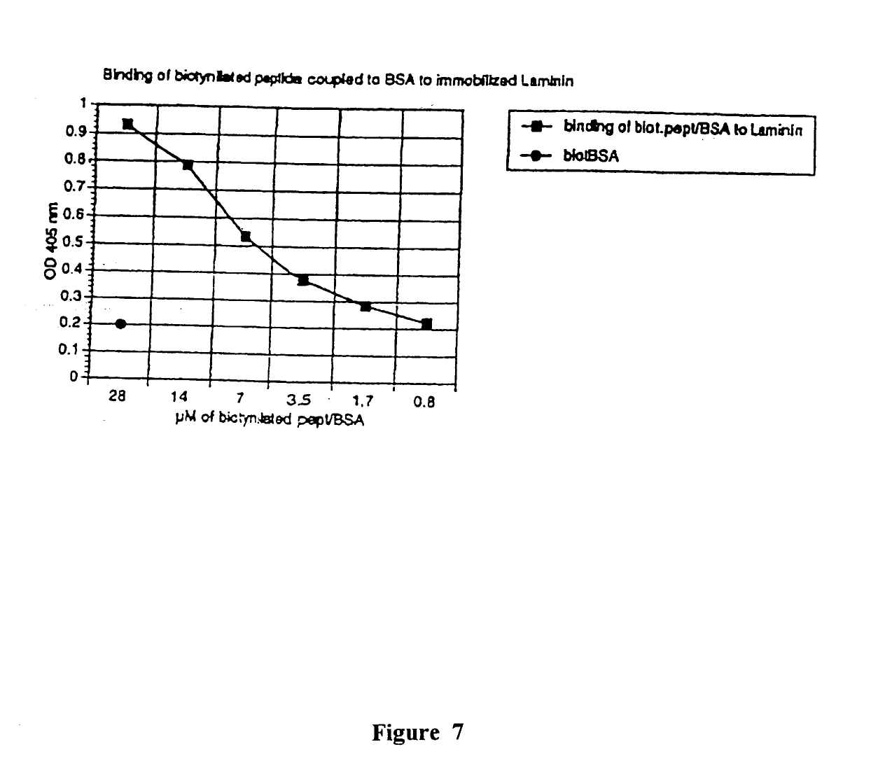

FIG. 7 shows the concentration-dependent binding of biotinylated complex to immobilized laminin, a binding partner of the HNK-1 carbohydrate. Binding of the biotinylated BSA, used as a control, was never observed.

Conclusions

We have screened a library of 15-mer peptide sequences expressed on the surface of filamentous phages for their ability to mimic the HNK-1 carbohydrate.

The peptide sequence FLHTRLFVSDWYHT (SEQ ID NO: 7) was synthesized and assayed for its ability to inhibit the binding of the L2/HNK-1 carbohydrate to its natural binding partner laminin or the binding of mAb L2-412 to HNK-1 glycolipids. We obtained an inhibitory effect of 30-35%. Our peptide shows an inhibitory effect comparable to that of the SO₃-sugar, which shows that the peptide behaves like the carbohydrate in these particular experimental conditions. Furthermore, the phage bearing another peptide used as a negative control never showed any inhibitory effect. The lack of complete inhibition of the mAb binding to the L2/HNK-1 glycolipids may be due to a multivalency-monovalency problem. The antibody is bivalent and the free peptide in monovalent. The peptide coupled to BSA might still act as a monovalent unit in this particular situation compared to the antibody. We demonstrate with these inhibition studies that the FLHTRLFVSDWYHT (SEQ ID NO: 7) peptide bind to the antigen combining site of the antibody, mimicking the L2/HNK-1 epitope recognized by L2-412. This conclusion was also confirmed with both the direct binding of the bioitynilated peptide-BSA complex to the L2-412 and to laminin and with the functional studies on neurite outgrowth from chicken motor neurons in vitro. A more detailed understanding of the molecular nature of protein carbohydrate interactions could influence the development of new therapeutic agents.

Binding to the L2/HNK-1 carbohydrate recognizing antibodies provides a good model to study the properties of mimics for biologically active carbohydrates. Peptides that compete effectively with the binding of the natural L2/HNK-1 carbohydrate to the antibody could also represent a step towards finding neutralizing compounds, which could prevent damage to nervous tissue by HNK-1 autoantibodies present in some neuropathies of the peripheral nervous system (Giese, K. P. et al., Cell 71, 565-576 (1992); Montag, D. et al. Neuron 13, 229-246 (1994)). Furthermore, HNK-1 binds to the P- and L-selections which are implicated in leukocyte-endothelial cell interactions also outside the nervous system. These interactions have been shown to play a role in immunopathological responses.

The peptide FLHTRLFVSDWYHT (SEQ ID NO: 7) (and a subfragment of it, FLHTRLFV (SEQ ID NO: 8) (see Example 2)), have been shown to at least in some respects mimic the L2/HNK-1 carbohydrate. Since the peptides are accessible through organic synthetic procedures, as well as in nucleic acids encoding such peptides, modified variants with altered amino acid sequences and analogs, including even unnatural amino acids, could be produced. Introduction of a sulfate group (perhaps on the N-terminal phenylalanine) might be a relevant modification, since this moiety is an important element of the natural HNK-1 carbohydrate. Testing carriers other than BSA might also lead to altered, improved or enhanced biological activity.

It would be of obvious interest to investigate the peptide mimics in vivo, particularly in connection with regeneration after lesions in the PNS or even in the CNS. After lesions in the CNS, the affected nerve fibers usually cannot regenerate and reconnect to their original targets. The regrowth of lesioned CNS fibers appears to be dependent on the CNS microenvironment encountered by the lesioned axons, as well as on the intrinsic growth potential of neurons (Kapffiammer et al, 1997; Schwab et al, 1996; Fawcett et al, 1998). In this respect, it is of special interest that the number and length of neurites growing out from motor neurons was significantly increased by the presence of the peptide mimic or the L2/HNK-1 carbohydrate itself (in its glycolipid form). Conceivably such effects would also be observed in vivo. Such activity might require coupling the peptide mimics to other carrier molecules, and appropriate routes of administration would have to be found. The immunogenicity of the peptides and the carriers, as well as the two in combination, would also have to be investigated. In the long term, it even seems possible that such a research program could lead to clinically useful substances.

Peptide mimics of carbohydrate have been successfully used in several research areas (Kieber-Emmons et al, 1998). Thus peptide mimics of carbohydrates have been tested as vaccines to induce immunity against group B streptococcus (Magliani et al, 1998) or neutralizing activity against HIV-1 (Agadjanyan et al, 1997). Peptide mimics of carbohydrates have also been applied in cancer research, where they were shown to induce an anti-tumor response in vivo (Apostolopoulos et al, 1998). The present work suggests that mimics of carbohydrates such as L2/HNK-1 could be used to promote regeneration in the nervous system after injury.

### EXAMPLE 2

### An Active 8-Mer Fragment of the Carbohydrate Epitope Mimic Peptide

As described above in Example 1, the phage peptides show a consensus sequence FLHTRLFV (SEQ ID NO: 8). To see whether the consensus itself might be active, the short 8 amino acid consensus sequence and a corresponding randomized form were also synthesized for comparison, both coupled to BSA (ANAWA AG, Switzerland). These were tested in combination with the 15-mer peptide and its corresponding randomized form, as shown in Table 3 below.

Peptide 15-15

F L H T R L F V S D W Y H T

Peptide 15-15 scrambled

L R S T W L D T Y F H V F H

Consensus peptide

F L H T R L F V

Consensus peptide scrambled

T V F H F R L L

Table 3. Peptide sequences and their scrambled forms. The scrambled forms were chosen manually, with attention being paid to the chemical characteristics of the side chain and the exact sequence of amino acids in the respective sequences.

### EXAMPLE 3

### The Carbohydrate Epitope Mimic Peptide Stimulates Neurite Outgrowth From Motor Neurons

Motor Neuron Experiments

To determine whether the peptide could also functionally mimic the HNK-1 carbohydrate, we cultured motorneurons of chick embryos in the presence or absence of the peptides coupled to BSA as described in the Materials and Methods section. The length and number of neurites were recorded for all the neurons with at least one process that was as long as the diameter of the cell body. From our experiment, we conclude that the motor neurons, in the presence of the peptides coupled to BSA, appear healthier and show a larger tendency to form a network between cells as compared to the control. In addition, the percentage of neurons bearing neurites up to a particular total length was significantly higher in the presence of the peptides coupled to BSA than in the control on BSA alone.

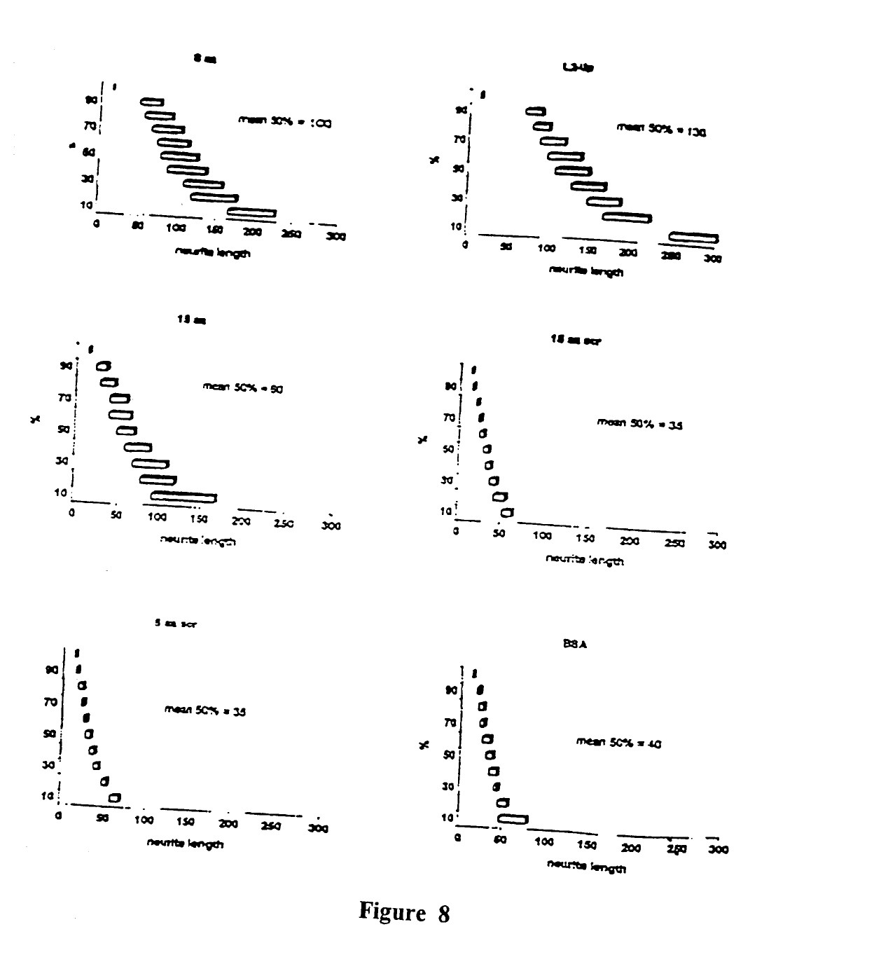

Motor neurons have been shown to extend significantly longer neurites when substrates containing either laminin or collagen are supplemented with the L2/HNK-1 glycolipid (Martini et al, 1992). To determine whether the isolated peptide could functionally mimic the L2/HNK-1 carbohydrate, motor neurons of chick embryos were cultured in the presence of BSA-peptide conjugate (8 and 15 amino acids coupled to BSA), scrambled BSA-peptide conjugate (8 and 15 amino acids scrambled coupled to BSA), or in the absence of these peptides. In this study, only collagen as “cosubstrate” was used; since the peptide had been shown to bind to laminin, inclusion of laminin might then have altered the substrate properties. When motor neurons were cultured for 24 hours on coverslips coated with the positive BSA-peptide conjugate, neurites were significantly longer than the neurites grown in controls without peptide, although there was variability among experiments (FIG. 8). In the positive control, motor neurons on L2-HNK-1-glycolipid coated coverslips showed neurite lengths in the same range as with the positive peptide. In contrast, motor neurons cultured on coverslips coated with either of the randomized BSA-peptide conjugate showed a neurite outgrowth similar to that observed on the BSA-coated coverslips used as negative control.

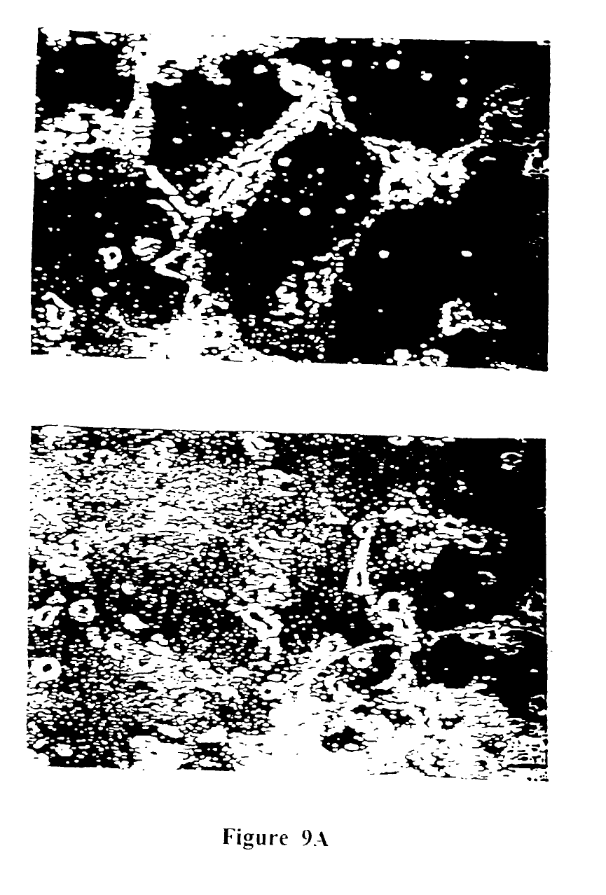

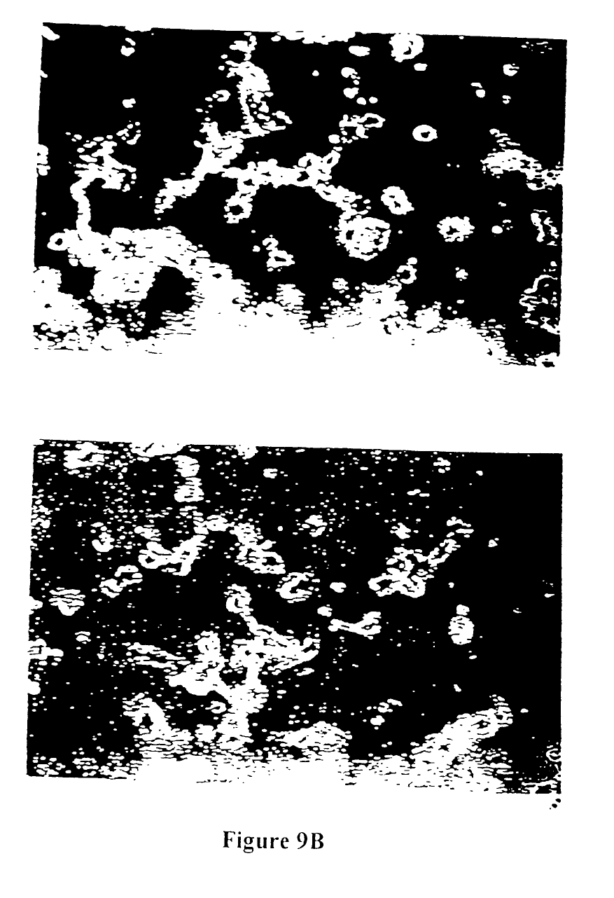

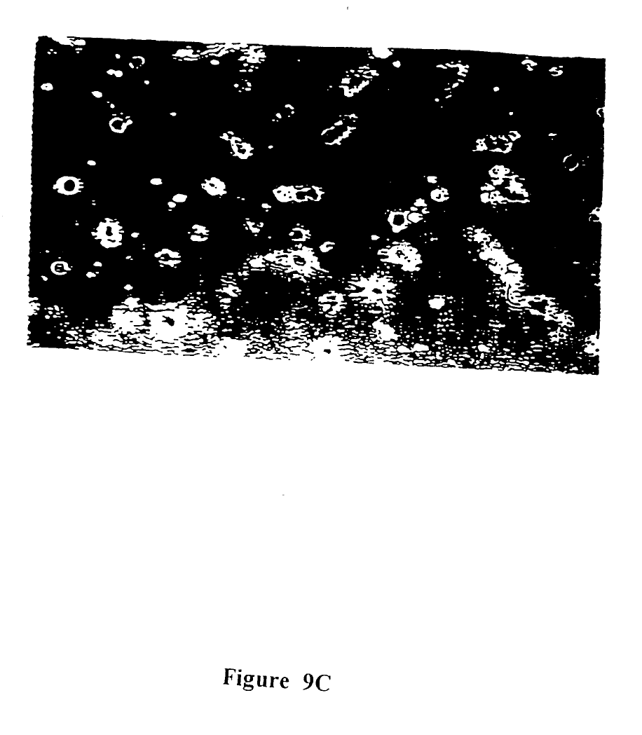

The neurites extended from motor neurons cultured on the short 8 amino acid sequence were significantly longer than the neurites obtained on control culture (BSA, randomized peptides). Similar results were obtained with the positive control, L2/HNK-1 glycolipid. The neurites extended in response to the 15 amino acid peptide sequence were shorter than those extended in response to the 8mer peptide, but still significantly longer than those obtained with the control consisting of BSA alone. By contrast, the neurites extended from motor neurons cultured on the randomized BSA-peptide conjugate were no longer than those on BSA alone. In these two cases, the network formed by the neurites also appeared less dense than that seen with the active peptides (FIGS. 9A-9C).

### EXAMPLE 4

### Effect on Neuronal Polarity

Another important point raised in this study is the possible role of the peptide in neuronal polarity. The concept of neuronal polarity implies that axons and dendrites are different. Chada and coworkers (Chada et al, 1997) have described differences between axon-like and dendrite-like processes, defining the axonal/dendritic polarity of the forebrain neurons. The cytoplasm of dendrite-like processes contained abundant polysomes throughout their length; in contrast, polysomes were not detected in the long axon-like processes in regions further than about 75 μm away from the soma. The dendrite-like processes showed a lower density of microtubules and neurofilaments than the axon-like processes. It has been shown for chick forebrain neurons (Chada et al, 1997) and for chick sensory neurons (Prochiantz et al, 1995) that mechanical tension initiates neurite elongation and that the addition to the culture medium of neurotrophic factors influences the development of fully polarized neurons (Lein et al, 1995; Prochiantz et al, 1995).

It was established that dendrite initiation is regulated separately from that of the axon by local factors. Thus, in vitro the number and length of dendrites from mouse cortical neurons were greater when the neurons were placed on glia (mostly astrocytes) from cortex, retina and olfactory bulb than when plated on glia derived from mesencephalon, striatum or spinal cord. Axonal growth was similar on all glial cells derived from the different CNS regions of early postnatal rats mentioned above (Le Roux et al, 1994). It was also suggested that the extracellular matrix molecules could be involved in these phenomenon. The hippocampal neurons from mice maintained on substrate containing tenascin were shown to develop a more polarized phenotype (Dorries et al., 1996; Lafont et al, 1994; Faissner at al, 1997; Lein et al, 1991; Lein et al, 1989). Together, these results highlight the importance of combinatorial effects.

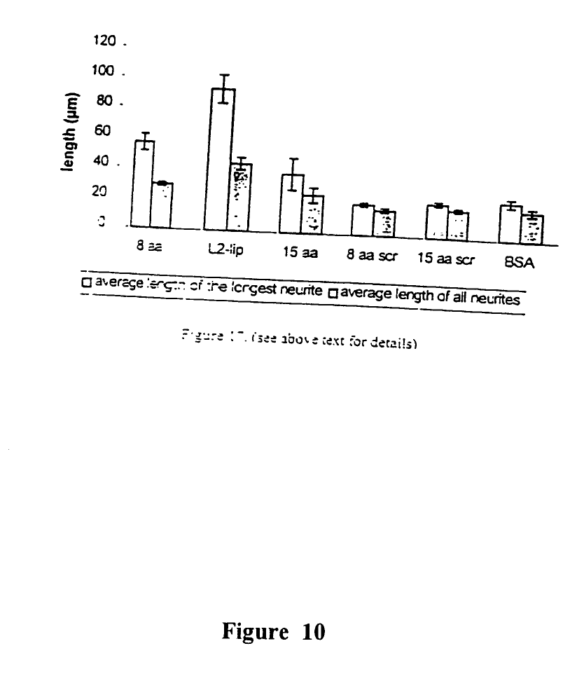

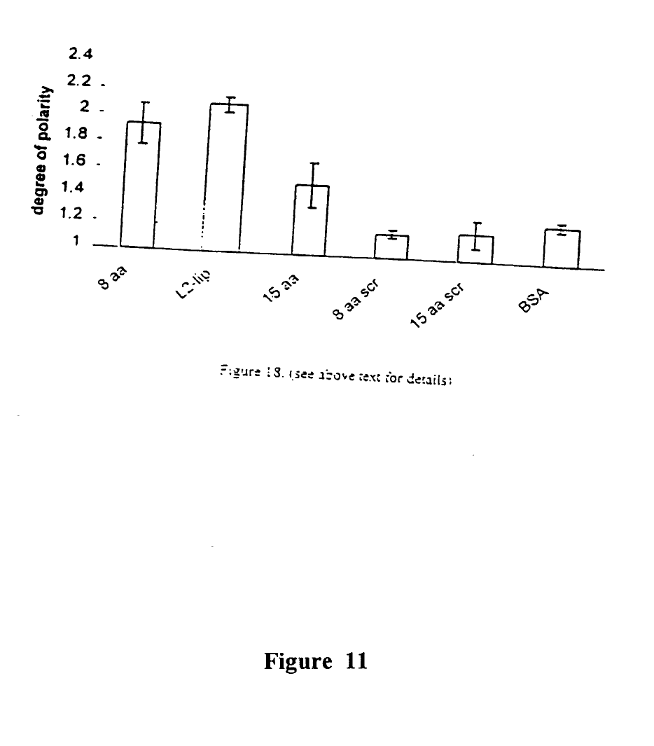

In this work, the polarization index was calculated based on the data provided by the IBAS analysis system. The degree of polarity was defined as the mean length of the longest neurite divided by the average length of all neurites, where the average length of all neurites was obtained by dividing the average total length by the average number of neurites. The higher the ratio, the more “polarized” the neuron, where polarized is defined to mean that one neuronal process (axon), which is the longest, predominates (FIG. 10 and FIG. 11). This index was found to be higher for the motor neurons cultured on either the FLHTRLFV peptide-mimic or on the L2/HNK-1 glycolipid compared to neurons in the control group (BSA or scrambled peptide) (FIG. 10 and FIG. 11). This is reminiscent of the in vivo situation, in which one process differentiates into an axon and becomes longer than the other processes. Interestingly, Lafont and co-workers (Lafont et al, 1994) showed that synthetic glycosaminoglycan-like (GAG-like) short sugar chains could influence neuronal morphology in vitro. Small synthetic heparin sulfate-like compounds have been shown to enhance axonal maturation of motor and cortical neurons, whereas dermatan sulfate-like compounds were primarily acting on the elongation of the dendrites from a subpopulation of cortical neurons with an established axon. Whether the FLHTRLFV peptide and L2/HNK-1 carbohydrate play a role in the differentiation of neurites into axons will be further investigated using specific markers for dendrites and axons. These could include microtubule associated protein 2 (MAP2) for dendrites and tau protein or neurofilament-H (NFH) for axons.

### EXAMPLE 5

### Outgrowth of Neurites From Dorsal Root Ganglion

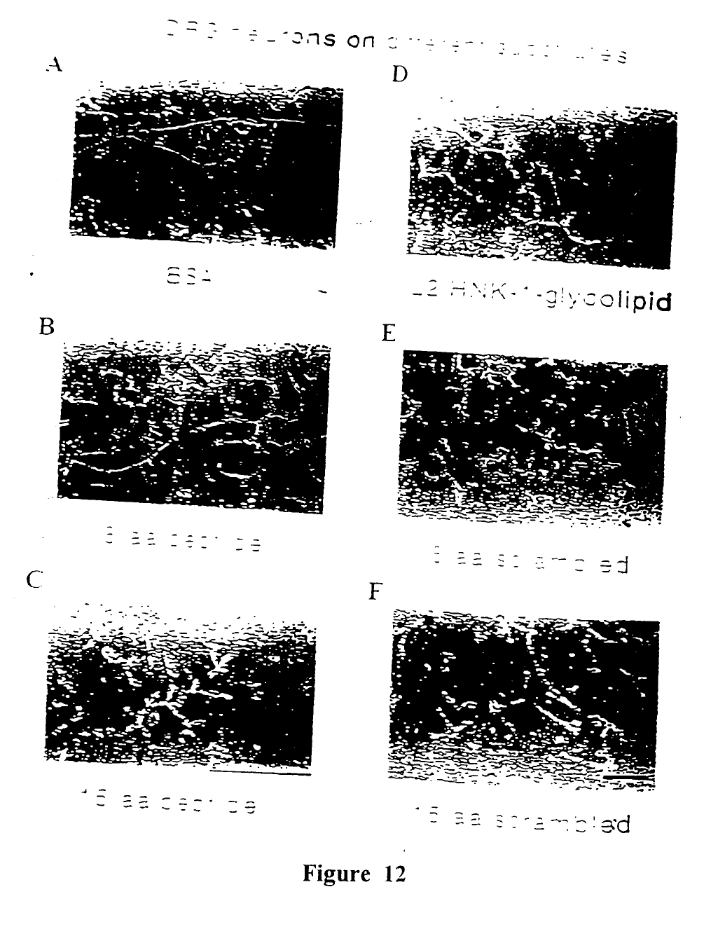

The L2/HNK-1 carbohydrate has been shown to be prominently expressed on the motor branch but scarcely on the sensory branch of the femoral nerve (Martini et al, 1992). It was shown that sensory neurons were indifferent to the presence of L2/HNK-1 in the substrate (Martini et al, 1992). It was therefore decided to assess neurite outgrowth from dorsal root ganglion neurons (i.e., sensory neurons) in the presence of the different BSA-peptide conjugates. Dorsal root ganglion neurons were obtained from 11-day-old-chicken embryos. Only the largest ganglia were taken and prepared. These experiments were repeated 3 times with essentially identical results. In contrast to the motor neuron cultures, where the difference in neurite lengths with various peptide substrates was striking, no obvious differences were seen with sensory neurons (results not shown/FIGS. 12A-12F). The neurite outgrowth observed on the BSA control was even better than that on the other substrates coated. Because of the poor definition of the neurites, their lengths could not be measured.

### EXAMPLE 6

### Use of Epitope Mimic Peptide in Immunocytochemistry

Investigations of ventral and dorsal roots and of femoral nerve of mice indicate that the large majority of the L2-412-immunoreactivity is associated with the ventral roots or with the motor branch, respectively. Thus, whether the peptide could bind to these same regions, which would be indicative of the presence of a potential receptor for the L2/HNK-1 carbohydrate was investigated. They were treated with (i) BSA-peptide conjugate, detected with and anti-BSA antibody followed by HRP-coupled goat anti rabbit serum (ii) and with, biotinylated BSA-peptide conjugate detected by HRP-streptavidin. Unfortunately, in neither case specific staining was detected. The L2-412 antibody, which was used as positive control for the immunostaining procedure (but not for the binding of the pept/BSA), gave strong staining of the ventral roots and of the motor branch of the femoral nerve. This also means that the L2/HNK-1 carbohydrate was present on these sections. It is possible that the amount of receptor was too low to be detected by binding of peptide, or that the receptor was already saturated with endogenous L2/HNK-1 carbohydrate. Alternatively, the peptide concentration or affinity was not high enough. This might be ameliorated with peptides coupled to other multivalent carriers.

Immunocytological localization of L2/HNK-1-carbohydrate receptors/recognizing molecules was also attempted on cultured motor neurons. Since the motor neurons grow significantly better on a substrate containing L2/HNK-1 carbohydrate, these cells should have a receptor for this carbohydrate. Biotinylated pept/BSA was incubated with fixed motor neurons that had been cultured on collagen. After washing, bound peptide was detected using HRP-streptavidin. A few cell bodies were stained when using the biotinylated 8aa pep/BSA. The stained cells were a minority compared to the unstained cells; both isolated cells or isolated group of cells were stained. No staining was observed in areas covered by a dense network of neurites. It is not clear whether this staining is significant, and these experiments certainly need more investigation and optimization. Nevertheless, the observation that only the consensus peptide, but not the scrambled peptide or BSA, showed staining (in three experiments) is of considerable interest.

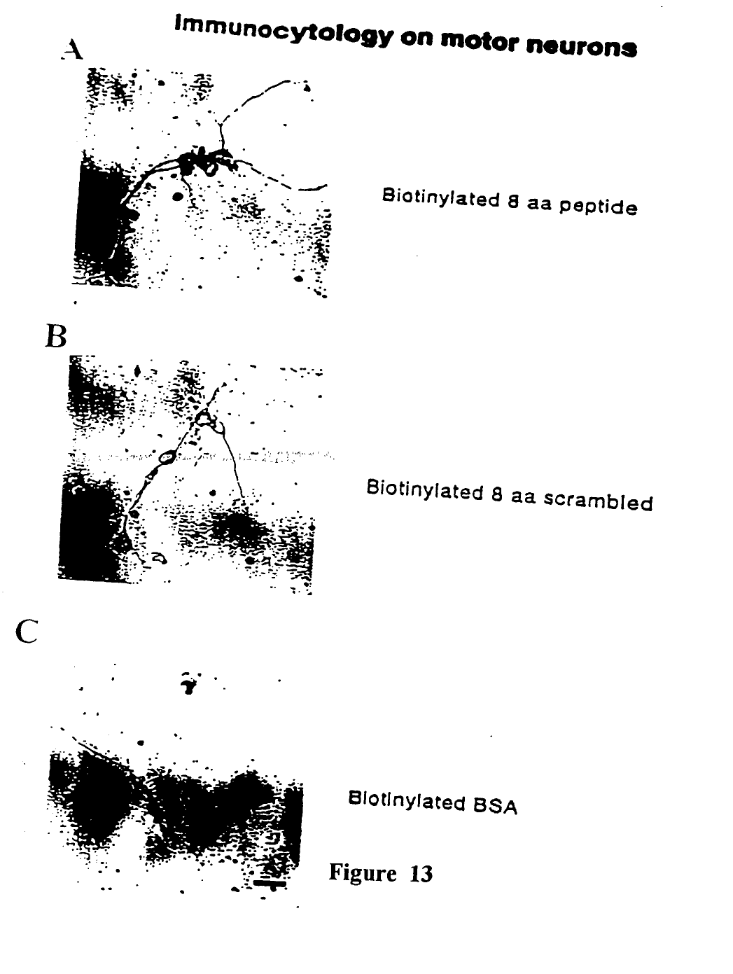

Immunocytological localization of a putative L2/HNK-1 carbohydrate receptor/recognizing molecule was attempted using the octapeptide coupled to biotinylated BSA. With cultured motor neurons, a few cell bodies could be stained, whereas no staining was seen when either biotinylated BSA or the biotinylated scrambled BSA-peptide conjugate were used (FIGS. 13A-13C). No staining was seen with cultured dorsal root ganglion neurons, consistent with their lack of response to either the peptide mimic or L2/HNK-1 carbohydrate in neurite outgrowth assays.

With motor neurons, the staining was observed only on isolated cells or on isolated groups of cells, but was never seen on cells involved in dense networks. Conceivably cells connected in a network have already produced as many neurites as required, and have down-regulated a receptor whose activation would otherwise stimulate neurite outgrowth. Single cells, on the other hand, might still extend new neurites to become attached to other cells and form a network.

### EXAMPLE 7

### Screening for Epitope Mimic Peptides With the HNK-1 Antibody

Having screened the phage library with the L2-412 antibody, an extension of that work would be to search for peptides specifically binding to the HNK-1 antibody. Since binding of the HNK-1 antibody requires the sulphate group in the carbohydrate antigen, such peptides might have distinct properties. A first screening was done with the amplified starting library. Several clones positive in binding to the HNK-1 antibody were found. However, they also bound to IgM.

In the initial screening with HNK-1, bound phage were eluted by pH shift, so that there was no differentiation between specifically and non-specifically bound phage. Therefore a screening was carried out wherein HNK-1 antibody is biotinylated with a coupling agent incorporating a disulfide bridge. The biotinylated antibody is pre-reacted with the streptavidin-coated tube, unbound antibody is washed off, and the immunotube is used for screening. Alternatively, phage are reacted with the biotinylated antibody in solution, and then the biotinylated complex is allowed to react with an immunotube coated with streptavidin. In either case, after washing away unbound phage, the bound phage are eluted by addition of dithiothreitol, which releases the antibody and the attached phage (Griffiths et al, 1994). Furthermore, these new screenings were done in the presence of mouse serum (12.5%). This provides a large excess of mouse IgM over the HNK-1 antibody, so that non-specific binding to the INK-1 antibody should be suppressed.

In some cases, when phage in solution were allowed to react with “pre-immobilized” antibody, a rise was obtained in the number of phage bound after the third Of the fourth round of selection. The clones tested bound to total mouse IgM as well. In a further experiment various procedures were compared in parallel: Phage were allowed to bind either to HNK-1 coated immunotube or to biotinylated HNK-1 in solution, and in the presence or absence of mouse serum. An enrichment was observed using the pre-coated antibody, and the selected clones again bound to total mouse IgM, although they also bound HNK-1 (and to a greater extent). It is interesting to note that the selected phage were also reactive to L2-412 antibody (again to a greater extent than they bound IgM to IgG).

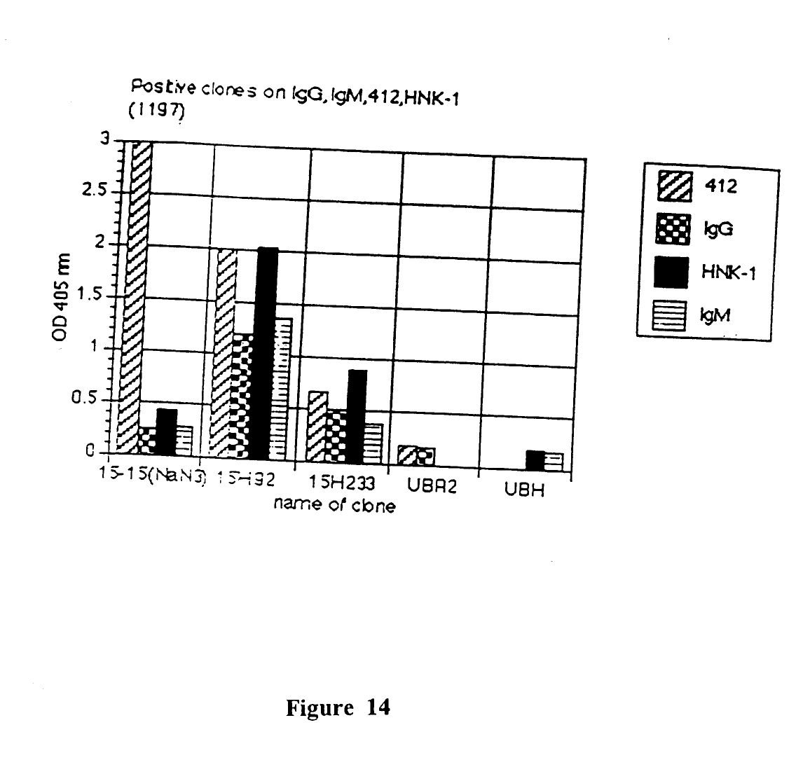

Comparative binding of the HNK-1 selected phage/positive clones versus the L2-412 selected 15-15 phage to bound L2-412 antibody, IgG, HNK-1, and IgM is shown FIG. 14. Supernatant (100 μl) from an overnight culture of bacteria secreting phade were incubated with coated antibodies (100 μl of 1 μg/ml useded for coating) and detected with HRP-coupled anti-phage antibody. Both of the HNK-1 selected phage 15H92 and 15H233 bind L2-412 and HNK-1, nearly to the same extent. The L2-412 antibody selected 15-15 phage does not bind HNK-1 antibody to a significant extent versus IgG or IgM.

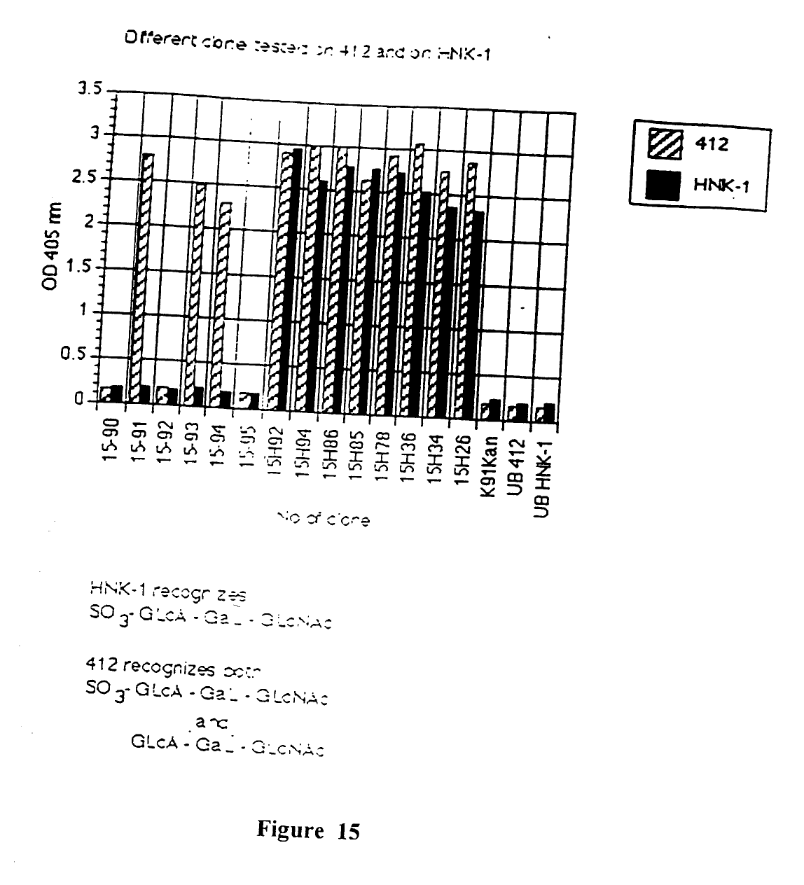

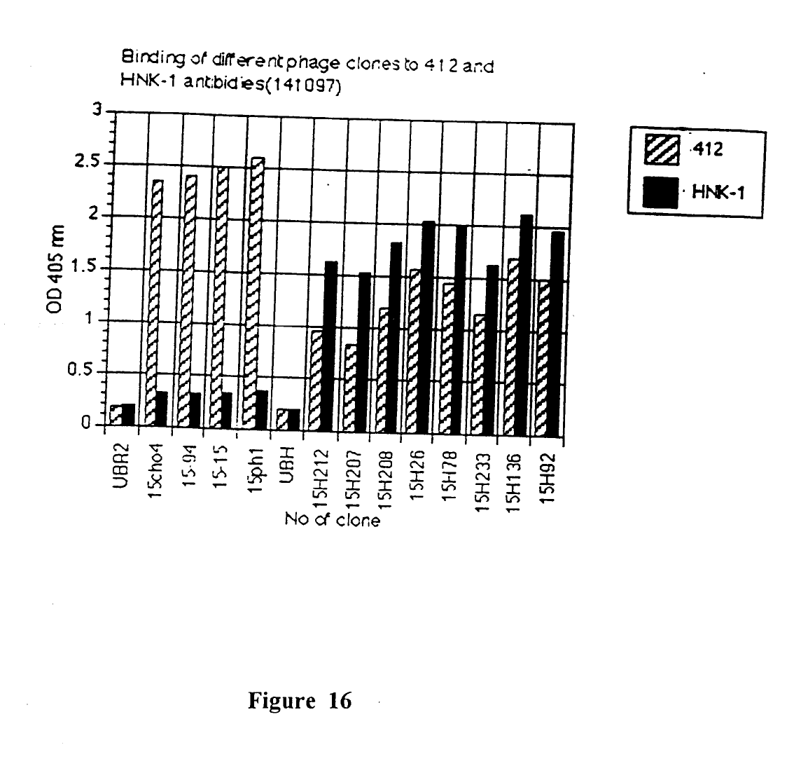

A further comparison of HNK-1 antibody selected clones versus L2-412 antibody selected clones is shown in FIGS. 15 and 16. The HNK-1 selected clones (all designated 15H#) all bind both HNK-1 and L2-412 antibodies to a significant extent, in some cases to an approximately comparable extent.

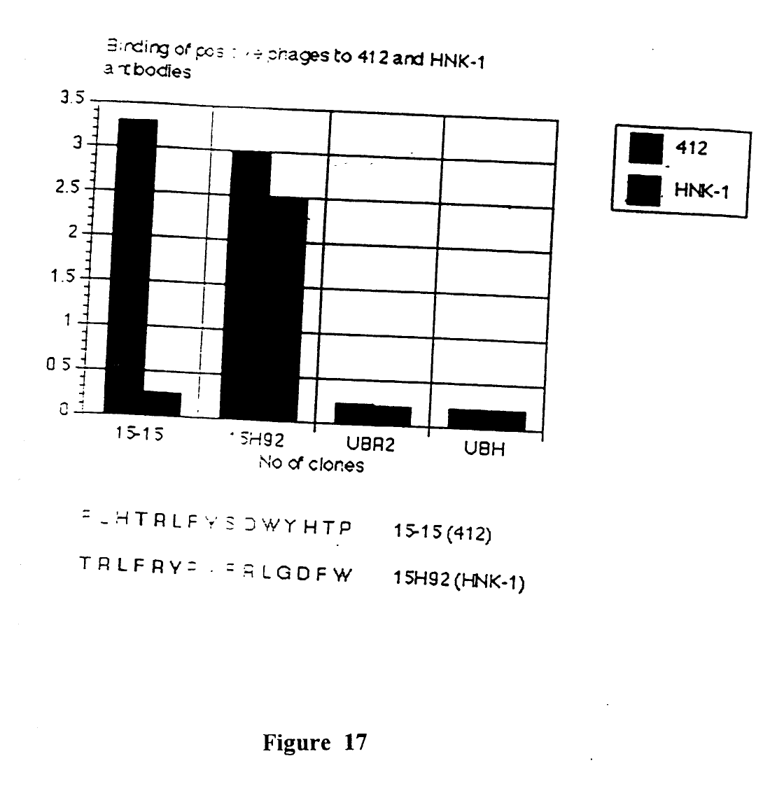

FIG. 17 depicts a direct comparison of 15-15, 15H92 and the controls UBR2 and UBH in binding 412 and HNK-1 antibodies. Control phage were picked at random from the unbound fraction after the first round of screening.

Ten of the HNK-1 selected phage were sequenced and the 15-mer sequences are shown in Table 4 corresponding to: 15H85, 92, 94 and 96 (SEQ ID NO: 34), 15H26 (SEQ ID NO:35), 15H36, 34 and 78 (SEQ ID NO: 36), 15H233 (SEQ ID NO: 37) and 15H 207 (SEQ ID NO: 38). As shown, the sequences of 15H85, 92, 94 and 96 are identical, as are those of 15H36, 34 and 78. For comparison, the sequences of certain L2-412 binder phage are also shown in Table 4, corresponding to: 15-3 (SEQ ID NO: 27), 15-15, 94, 91, 97, and 9 (SEQ ID NO: 28), 15-81 (SEQ ID NO: 31), 15-84 (SEQ ID NO: 32), 15-cho4 (SEQ ID NO: 30), and 15 PHI (SEQ ID NO: 33). A consensus sequence of TRLFR V/F (SEQ ID NO: 39) is found in eight of the phage clones. Interestingly, the sequence shows similarity and homology to the consensus 8 mer of the L2-412 phage, FLHTRLFV (SEQ ID NO: 8), particularly containing the sequence TRLF(R)V (SEQ ID NO: 40) and TRLF (SEQ ID NO: 41) which is conserved universally and compressed in many of the L2-412 and HNK-1 binders.

TABLE 4
COMPARISON OF SEQUENCES

HNK-1 Positive Binders
T R L F R V P V F R L G D F W /15H85, 92, 94, 96
T R L F R F L S S V W G L L A /15H26
T R L F R V P V L P S G V T S /15H36, 34, 78
S L A P Y S L R I F V L F G G A /15H233
S L A R S F H A Y F R H T L V G P /15H207
412 Positives Binders
T F T R V V T D V Y R G R L S /15-3
F L H T R L F V S D W Y H T P /new15-15, 15-94,

91, 97, 9

A F L H T R L L F R I V S Y S G P /15-81
A F L H T R L L F R N G I I L R P /15-84
A F L H T R L F V S D W Y H T P G /15-CHO4
A F L H T R L F V S D G I N S G P /15PH1

### EXAMPLE 8

### CD4 Protein Contains Consensus L2/HNK-1 Epitope Binding Sequence

Laminin is a self-aggregating, multifunctional glycoprotein, consisting of three polypeptide chains α1, β1 and γ1. Laminin is known to recognize the L2/HNK-1 carbohydrate, and such carbohydrate is implicated in cell-to-laminin adhesion. Cell-to-laminin adhesion is mediated by direct binding of the L2/HNK-1 carbohydrate to the G2 domain of the terminal globular domain of the laminin α1 chain. Hall et al has reported testing a variety of G2 domain-derived synthetic peptides for their ability to inhibit L2/HNK-1 binding to laminin, and has isolated the competitive binding to a single peptide (Hall et al., Glycobiology 5, 435-441 (1995). This peptide, KGVSSRSYVGCIKNLEISRST (SEQ ID NO: 51) bound to the L2/HNK-1 carbohydrate in a concentration-dependent manner and inhibited HNK-1-mediated neural cell adhesion to laminin.

The laminin peptide sequence was used to search the publicly available sequence database and a group of proteins possessing homologous sequences were identified (SEQ ID NOS: 52-57) (TABLE 5). A consensus L2 binding protein sequence was also determined (SEQ ID NO: 58)(TABLE 5).

TABLE 5

| | Amino | |
|---|---|---|
| | Acid | |
| Protein | No. | Sequence |
| EHS-Laminin | 2431- | K G V S S R S Y V G C I K N L E I S R S T |
| A (G2) | 2451 | |
| Human | 2480- | R G V T T K S F V G C I K N L E I S R S T |
| Laminin | 2508 | |
| A (G2) | | |
| Merosin (G2) | 506- | P E V N L K K Y S G C L K D I E I S R T P |
| | 525 | |
| L-Selection | 93- | K V E G V W T W V G T N K S L T E E A K |
| | 112 | |
| CD4 | 244- | R A S S S K S W I T F D L K N K E V S V K |
| | 264 | |
| PO | 64- | G T F K E R I Q W V G D P S W K |
| | 79 | |

L2 Binding Protein Consensus Sequence:

(R,K)X{4}(R,K)X{1}(Y,W,F)X{0,5}(R,K)X{0,3}(E,D)X{0.3}(R,K)

(R,K)=either R or K X{0,5}=between 0 and 5 amino acids

(Y,W,F)=either Y,W or F X{0,3}=between 0 and 3 amino acids

The proteins include already recognized L2/HNK-1 interacting molecules, including merosin, L-selectin, and PO. In addition, the CD4 protein, present on lymphocytes and recognized as interacting with HIV virus and required for HIV infection of human cells, contains a homologous sequence at amino acids 244-264, corresponding to RASSSKSWITFDLKNKEVSVK (SEQ ID NO: 56). This amino acid sequence is located in the extracellular domain of the CD4 protein, between the C-like domain and the transmembrane domain.

### EXAMPLE 9

### CD4 Protein Binds L2/HNK-1 Glycolipids

To confirm whether the 244-264 region of CD4 was capable of binding to L2/HNK-1carbohydrate, a 21-mer peptide corresponding to this amino acid sequence was synthesized and tested in a series of binding and competition experiments. The 15 methods used in the following set of experiments use comparable procedures as described in the Materials and Methods section, except where noted below, however, CD-4 peptide was used.

The ability of isolated L2/HNK-1 glycolipid to bind to immobilized CD-4 peptide was tested. CD-4 peptide was immobilized in water at different concentrations and dried onto microtiter plates overnight. The microtiter plates were blocked with 1% BSA in PBS, incubated with 3 ug/ml L2 glycolipid for 1.5 hours at room temperature, and washed with PBS. Bound L2 glycolipid was detected with L2-412 antibody followed by HRP-linked secondary antibody. The reslts (not shown) demonstrated that CD-4 peptide binds L2 glycolipid in a concentration dependent manner.

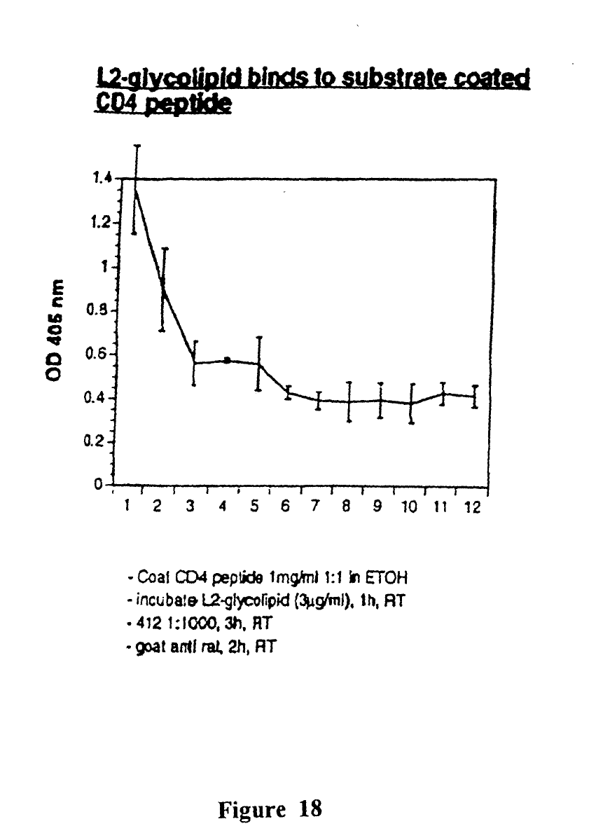

It was determined whether L2 glycolipid could bind substrate coated CD-4 peptide. CD4 peptide was coated onto microtiter plates by coating with serial dilutions of CD4 peptide (1 mg/ml 1:1 in EtOH) and allowed to dry overnight. The wells were incubated with L2-glycolipid (3 μg/ml) for 1 hour at RT, L2-412 antibody (1: 1000) then added and further incubated for 3 hours at RT. Goat anti-rat antibody was then added, incubated 2 hours at RT and detected by HRP-linked secondary antibody. As shown in FIG. 18, L2 glycolipid binds to CD4 peptide in a concentration-dependent manner.

The CD-4 peptide was conjugated to Ovalbumin (by similar methods as for BSA conjugation described in Materials and Methods) to generate CD4-OV, and binding experiments were performed to assess binding of conjugated peptide to laminin and L2 glycolipid. In particular, it was necessary to confirm that CD4 peptide and laminin compete directly in binding to L2/HNK-1 glycolipids. In this set of experiments, microtiter plates were coated with serial dilutions of either CD4-OV or laminin (each 10 μg/ml 1:1 in EtOH, allowed to dry overnight). The coated plates were then incubated in the following combinations: (a) CD4-OV coated plates with L2 glycolipid (3 μg/ml, 1 hr, RT); (b) laminin coated plates with L2 glycolipid (3 μg/ml. 1 hr, RT); and (c) laminin coated plates with CD-4-OV (1 μg/ml) and L2 glycolipids (3 μg/ml) for 1 hr at RT. Similar to the previous set of experiments L2-412 antibody was added (1:1000, 3 hr, RT), followed by HRP-conjugated-goat anti-rat antibody (2 hrs, RT) and detection by HRP-linked secondary antibody. The results (not shown) confirmed that CD4 peptide and laminin compete directly in binding to L2/HNK-1 glycolipids. In (a) and (b), L2-glycolipid bound in a concentration dependent manner to either CD4 peptide or laminin. In (c), CD4 peptide competed with laminin for L2-glycolipid binding.

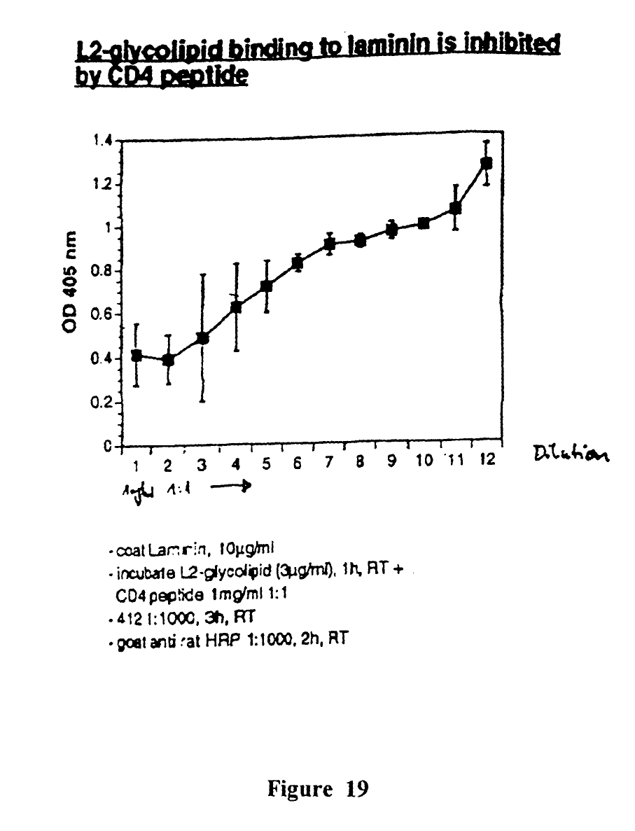

To further assess whether the CD-4 peptide could inhibit L2 glycolipid binding to laminin, a fixed amount of L2 glycolipid was incubated with immobilized laminin, in the presence of varying concentrations of CD-4 peptide. Microtiter plates were coated with serial dilutions of laminin (each 10 μg/ml 1:1 in EtOH, allowed to dry overnight). The coated plates were then incubated with L2 glycolipids (3 μg/ml) for 1 hr at RT plus CD4 peptide (1 mg/ml 1:1 in EtOH). Similar to the previous set of experiments L2-412 antibody was added (1:1000. 3 hr, RT), followed by HRP-conjugated-goat anti-rat antibody (2 hrs, RT) and detection by HRP-linked secondary antibody. As shown in FIG. 19, CD-4 peptide competes with L2 glycolipid binding to immobilized laminin in a concentration-dependent manner.

Materials and Methods

Materials

The 15-mer peptide library and the E.coli K91Kan cells used were kindly provided by G. Smith, Division of Biological Sciences, University of Missouri, Columbia. The 15-mer library was constructed in the vector fUSE5, a derivative of the filamentous phage fd-tet (Scott et al, 1990). This vector carries a tetracycline resistance gene allowing for selection. The filamentous phage do not kill their host; thus the infected cells become tetracycline resistant, continue to grow and secrete progeny particles.

The E. coli strain K91 Kan (also from G. Smith) is a lambda⁻ derivative of K38 (Lyons et al, 1972), has a chromosomal genotype thi and carries a kanamycin-resistance gene (mkh) (Smith et al, 1993; Yu et al, 1996). Peptidesand peptide (10 mg) coupled to SPDP-activated BSA (60 mg) via C-terminal cysteine, were ordered from ANAWA AG, 8602 Wangen, Switzerland. Tetracycline and Kanamycin were purchased from Sigma. L2/HNK-1 glycolipids were purified from beef cauda equina by B. Becker in our laboratory. Sulfated sugars, SO₃-GlcA-Gal-allyl, were kindly provided by N. Nifant'ev, Zelinsky Institutre of Organic Chemistry, Russian Academy of Sciences, Moscow.

Antibodies

Characterization and purification of the monoclonal antibody (mAb L2-412), raised in rats and recognizing the HNK-1 carbohydrate has been described by Noronha, A. et al., Brain Res. 385, 237-244 (1986)). The L2-412 antibody has been deposited with the DSMZ—Deutsche Sammlung Von Mikroorganismen und Zellkulturen GmbH, Mascheroder Weg 1b, D-38124 Braunschweig, Germany, under the Budapest Treaty, and is designated ______. HNK-1 antibody is available as TIB200 from the American Type Culture Collection (ATCC). Polyclonal rat IgG and HRP-Streptavidin were obtained from Sigma (USA). HRP/anti-M13 polyclonal antibody was purchased from Pharmacia Biotech. Horseradish peroxidase (HRP)-conjugated secondary antibody directed against rat IgG was obtained from Jackson Immunoresearch.

Amplifying the Starting Library

The primary library encoding the 15 mer peptides was amplified based on the Smith procedure (Smith et al, 1992) as follows:

The night before the cells were needed, 2 ml of LB medium (g/L Bacto-Tryptone, 5 g/L NAcl, 5 μL yeast extract), containing 100 μg/ml kanamycin, were inoculated with K91Kan cells and shaken overnight at 37° C. A IL flask containing 100 ml of Terrific Broth was prepared (12 g Bacto-Tryptone, 24 g yeast extract, 5.04 g glycerol (4 ml) added to 900 ml of water and autoclaved in 90 ml portions; 10 ml of potassium phosphate buffer (0.17M KH₂PO₄, 0.72M K₂HPO₄, no pH adjustment required) were added to each 90 ml portion bef ore use).

The 100 ml Terrific Broth were inoculated with 1 ml of the overnight culture of K91kan cells and shaken vigorously until the OD₆₀₀ of a 1:10 dilution reached 0.2. Shaking was then slowed down for 10 min to allow F-pili to regenerate and 10 μl of the starting library was added to the flask; slow shaking was continued to allow for adsorption. The culture was then transferred to 1 L of LB containing 0.22 μg/ml tetracycline and allowed to shake vigorously for 35 minutes at 37° C. The tetracycline concentration was adjusted to 20 μg/ml, and an aliquot was taken for determination of the titer. The phage were titered (recovered titer) by plating infected cells on tetracycline medium and counting the number of tetracycline resistant colonies. An infectious unit defined in this way is called a transforming unit (TU) and the infectivity is the ratio of number of TU's to number of physical particles. Typically, an aliquot of 50 μl of the culture was removed and diluted with LB containing 0.2 μg/ml tetracycline (dilution range was 10³-10⁵). An aliquot of 200 μl of each dilution were spread on an agar-plate containing 40 μg/ml tetracycline and 100 μg/ml kanamycin, incubated overnight at 37° C. The colonies were counted on the next day. At this stage, the titer of tetracycline resistant colonies should be about 10⁷/ml. The remainder of the culture was shaken vigorously overnight.

The next morning the doubly cleared supernatant obtained after 2 steps of centrifugation (4000×g, 10 min, 4° C. and 10'500×g, GSA, 10 min, 4° C.) was precipitated overnight at 4° C. by adding 0.15 volume of PEG/NaCl solution (16.7% polyethylene glycol in 3.3 M NaCl solution). The precipitated phages collected after centrifugation (10'500×g, GSA, 40 min, 4° C.) were dissolved in 10 ml of TBS (50 mM Tris-HCl pH 7.5, 150 mM NaCl) and a second precipitation was carried out by adding 0.15 volume of the PEG/NaCl solution to the phage suspension and incubating for 1 hr on ice. At this stage, a heavy precipitate should be evident.

The pellet obtained after centrifugation (14'500×g, SA600, 10 min, 4° C.) was redissolved in 10 ml TBS and transferred into a tared vessel containing 4.83 g CsCl. The vessel was retared and TBS was added to a net weight of 10.75 g. This should give 12 ml of a 31% w/v solution of CsCl (density 1.30 g/ml); the solution was centrifuged 48 hrs at 150'000×g at 5° C. in a SW41 rotor (Beckman). With the help of a strong visible light source, a faint bluish non-flocculent band (containing the amplified phages) was visible above a narrow flocculent opaque white band (probably deriving from PEG). The phage band was collected by first aspirating slowly the fluid overlying the phage band and then, using a pipette, the phage band was withdrawn avoiding as much as possible the flocculent band underneath. The phage band was then delivered to a 26 ml polycarbonate centrifuge bottle, which was filled to the shoulder with TBS and centrifuged in a Ti70 rotor (279'000×g, 4 h, 5° C.) and resuspended in 2 ml TBS per 1 L of culture. Phages can be stably stored in this form in a refrigerator.

The amplified library was then titered (final titer) as follows: several dilutions of phage were prepared in TBS/gelatine (0.1 g gelatin in 100 ml TBS) covering the dilution range from 107 to 10¹⁰. Then 10 μl of each of these dilutions were used to infect 10 μl of K91kan cells prepared as described at the beginning of this section and each dilution mixture was incubated 15 min at room temperature (RT) to allow phage to infect the concentrated cells. One ml of LB containing 0.2 μg/ml tetracycline was added and incubated 30 min at 37° C. in a shaker-incubator. The infected cells were then spread (200 μl) on an agar plate containing 40 μg/ml tetracycline and 100 μg/ml kanamycin as described above (recovered titer).

Screening Procedure

A. Direct Binding

The phage library was panned using Immunotubes (Nunc., Maxisorb) coated with mAbL₂-412. The tubes were coated by incubating overnight at 4° C. with antibody L2-412 at 10 μg/ml protein in PBS (1 ml total volume) for the first round and 1 μg/ml for the second and third round of screening. After blocking 2 hours with Blotto (5% non-fat dry milk, 0.05%(v/v) Tween 20 in PBS) at 4° C., 10¹¹ transforming units (in 250 μl volume) of the phage library per immunotube were allowed to bind 1 hour at 37° C. in a rotating chamber. For the second and third rounds, the phages were preincubated 1 hour with 100 μg/ml of rat IgG before being added to the immunotube, in order to decrease the number of non-specific binders. After recovery of the unbound phages (from which the negative control phage was chosen), the tubes were washed 10 times with PBS-0.05% (v/v) Tween 20 and eluted with 0.1 M Glycine pH 2.2 (0.5-1 ml total volume), 10 min. at 4° C. Eluted phages were neutralized with 1.5M Tris pH 9 and then used to infect 0.5-1 ml of log phase E. coli K91Kan cells 15 min at room temperature. The infected bacteria were transferred to 20 ml of LB containing 0.2 μg/ml tetracycline, and after removing an aliquot for determination of the titer (recovered titer), allowed to grow overnight as described in the previous section. The amplified eluate was then twice centrifuged (10 min, 3600×g and 10 min, 14'500×g, SA600) and the final supernatant was precipitated with 0.15 volume of PEG/NaCl overnight at 4° C. The phage was pelleted (15 min. 14'500×g, SA600) and dissolved in 1 ml PBS by pipetting and vortexing, microcentrifuged 1 min. to pellet insoluble matter, and PEG-precipitated again for at least 1 hr at 4° C. A heavy precipitate should be visible at this stage. The pellet obtained after 10 min. microcentrifugation was finally dissolved in 200 μl of PBS containing 0.02% azide. This amplified eluate can be stored and kept at 4° C. The library was subjected to three rounds of amplification and selection.

The same procedure was used for the HNK-1 screening with HNK-1 antibody, except that a 100-fold excess of mouse IgM was included to decrease non-specific binding.

The phage were titered (final titer) as described. The colonies were counted on the next day and the yield of the screening was calculated by dividing the recovered titer by the titer (input) of the previous round.

B. Screening With Biotinylated Antibody

Two procedures were used to accomplish this screening, both following protocols of G. Smith (unpublished protocols). The HNK-1 antibody was biotinylated as described below using NHS-SS-biotin. NHS-SS-Biotin links the biotin to the protein via a disulfide bridge, in order to allow the biotin group to be subsequently removed by incubation with dithiothreitol (DTT). The L2-412 antibody was similarly biotinylated as described below. In procedure A, the biotinylated antibody is first allowed to bind to a streptavidin coated immunotube, which is then subsequently used to pan the phage input. In procedure B, the biotinylated antibody is preincubated with the phage in solution, and the reaction mixture is allowed to bind (a few minutes) to the streptavidin-coated immunotube.

In procedure A, the immunotubes were coated with 10 μg/ml streptavidin in PBS, 1 ml total volume (wet the entire surface of the tube), overnight at 4° C. on a rotator. Streptavidin was discarded and the tube was filled with blocking solution, PBS containing 0.5% (w/v) BSA, for 2 hrs at 4° C. After washing 6 times with PBS-0.05% (v/v) Tween 20 (PBS-T), the biotinylated antibody was added. Typically, 3 μg of the biotinylated HNK-1, or 5 μg of the biotinylated L2-412 antibody were added in 400 μl of the blocking solution. The antibody was allowed to bind for at least 2 hrs (or overnight) at 4° C. on the rotator. After washing 6 times with PBS-T, 10¹⁰ phages from the 15-mer starting library, in 400 μl of blocking solution, were allowed to bind to the respective antibody-coated immunotube for 4 hr at 4° C. on the rotator. In procedure B, during coating of the immunotubes 10¹⁰ phage were preincubated overnight with 3 or 5 μg of the biotinylated HNK-1 or L2-412 antibody, respectively. The biotinylated antibody was then allowed to bind to the coated immunotube for 10 minutes at 4° C. on the rotator. In both procedures, the tubes were then washed 10 times, then phage-antibody complexes were eluted with 20 mM DTT (0.5 ml volume) in PBS 1-5 min. at room temperature. Amplification and titering were performed as described above. The library was subjected to four rounds of amplification and selection.

ELISA Screening

A. Direct Binding for Detection of Positive Clones

Individual colonies resistant to tetracycline and kanamycin were grown in LB containing 20 μg/mil tetracycline in 96-wells plates (Nunc) overnight at 37° C. (300 μl/well), then centrifuged 10 minutes at 3000 rpm in Jouan centrifuge and the supernatant (100 μl) was incubated for 2 hr in another 96-well plate previously coated with mAb_{L2}-412 (100 μl, μg/l ml overnight at 4° C.) and blocked by incubation for 2 hours with PBS-0.5% (w/v) BSA. After washing 5 times, the binding of the phages was detected by incubation with HRP-conjugated anti-M13 antibody (Pharmacia, Biotech.) for 1 hour at a dilution of 1:2000. The peroxidase reaction was started by the addition of 100 μl developer containing 0.01% hydrogen peroxide and 0.1% (w/v) 2,2′-azino-bis(3-ethylbenzthiazoline-6-sulfonic acid)-diammonium salt (ABTS, Boehringer Mannheim) in HRP buffer (0.1M sodium acetate, 0.05M NaH₂PO₄, pH adjusted to 4.2 with acetic acid). The absorbance of the colored reaction product was determined at 405 nm in a Multiscan TitertekPlus (Flow, Switzerland). In parallel, each clone was also tested on 96-well plates coated with rat IgG, (100 μl, 1 μg/ml in PBS and identically blocked for 2 hours). Bacteria producing the selected binding clones (named positive phage), that were positive binders for the mAb_{L2}-412 but did not bind to rat IgG were streaked on an agar plate containing LB medium with 40 μg/ml tetracycline and 100 μg/ml kanamycin. Two individual colonies were picked and re-assayed for positivity towards mAb L2-412. Positive single colonies were stored in 40% glycerol at −80° C.

B. Competition Binding

Microtiter plates (Nunc) were coated with the L2/HNK-1 glycolipids (50 μl, 1 μg/ml, dissolved in EtOH) and allowed to dry overnight. While blocking the wells for 2 hours with 0.5% (w/v) fatty-acid-free BSA in PBS, a limiting concentration of ^{L2}-412, previously determined, was pre-incubated with successive 2-fold dilutions of the inhibitor, starting at a concentration of 2.2 mM for the free peptide, 5 mM for the SO₃ sugar and 10¹² positive and negative phages (the negative phages were cloned from the unbound fraction of the first round of screening). The pre-incubated mixture was then added to the well in 100 μl and incubated for 1 hour at RT. After washing 5 times with PBS-0/05% (v/v) Tween 20, the binding of mAb L2-412 was detected by incubation with HRP-conjugated goat anti-rat IgG for 1 hour, followed by the color reaction described earlier. The percentage of inhibition of the binding of mAb L2-412 to the substrate in the presence-of the inhibitor was calculated with reference to the control value obtained in the absence of inhibitor (0% of inhibition).

C. Inhibition of Binding

Microtiterplates were coated overnight at 4° C. with laminin (Gibco/BRL), (10 μg/ml, 100 μl), or mAbL₂-412 (1 μg/ml, 100 μl) in PBS. All the following reaction steps were carried out at room temperature. After blocking with PBS+0.5% (w/v) BSA, 50 μl of successive 2-fold dilutions of peptide coupled to BSA (ANAWA Ag, Switzerland) starting at a concentration of 30 μM was added for 1-2 hours at RT. Then a limiting number of phages bearing the peptide of interest, previously determined, was added and incubated for another hour. The bound phages were detected with HRP/anti-M13 antibody as described in the ELISA screening section. The analogous experiment was done with immobilized L2-41.2 instead of laminin, the peptide coupled to BSA competing with the binding of positive phages to the antibody L2-412.

D. Direct Binding to Laminin

Microtiter plates were coated with 1001 μl of mAb L2-412 or laminin as described above and 100 μl of biotinylated peptide coupled to BSA was added starting at a concentration of 30 μM, incubated 2 hours at room temperature, and detected with HRP-streptavidin.

DNA Sequencing

Positive clones, toothpicked from frozen glycerol stocks, were grown overnight at 37° C. in LB containing 20 μg/ml tetracycline. Single stranded DNA was purified as described by G. Smith (1992) using the double-spin method, sequenced with the Thermo Sequenase cycle sequencing kit (Amersham), and loaded on an automated sequencer (B10 Genetic Analyzer, Applied Biosystems Inc.).

Biotinylation

Biotinylation of the HNK-1 antibody, BSA and the peptide coupled to BSA was done using Sulfo-NHS-biotin (Pierce) according to the manufacturer's instructions. A molar ratio of 10 to 1 was used for the antibody and 5 to 1 for BSA or the peptides coupled to BSA. The biotinylated product was dialysed overnight against PBS at 4° C.

Neurite Outgrowth Experiments

Preparation and Culture of Motor Neurons

Cover slips were sterilized by baking them overnight at 160° C. and coated by an overnight incubation with polyornithine (Sigma, 1.5 μg/ml in water) at 4° C. The cover slips were then washed 3 times with water and further coated with test substances as follows: 1) The BSA-peptide conjugates were dissolved at 100 μg/ml in PBS, sonicated 1 min with a table sonicator and centrifuges in a microfuge for 20 min at maximum speed. The protein concentration of the supernatant was determined each time by the method of Bradford (Bradford et al, 1976). Then 120 p 1 complex was mixed with 280 μl of collagen solution (20 μg/ml collagen in PBS) and 100 μl were applied on each cover slip overnight at 4° C.; 2) As a negative control, untreated BSA was used in place of the peptide-BSA complex; 3) The glycolipids carrying the L2/HNK-1 carbohydrate were dissolved in ethanol at a concentration of 10 μg/ml, and 80 μl were added to 1 ml of the collagen solution described above. A volume of 100 μl was used for coating. Cover slips were placed in quadruplicate in a 24-well plate (NUNC), and finally washed 3 times before the cells were plated (the cover slips were never allowed to dry).

Motor neuronal cells were prepared as described by Arakawa (1990) from spinal cord of 6-day old chick embryos dissociated in 1 ml of ice cold solution containing 0.05% DNAse 1 (Sigma), 0.1% BSA in L-15 medium (Life Technologies). Cells were layered on 2 ml of 6.8% Metrizamide (Fluka) in L-15 and centrifuged 15 minutes at 500×g, 4° C. Cells collected from the Metrizamide/medium interface were diluted in 5 ml L-15 and loaded on a 4 ml cushion of BSA (4% BSA in L-15) and centrifuged 10 minutes at 300×g, 4° C. The pellet was resuspended in 0.5-1 ml of complete medium ((22 mM NaHCO₃, 22 mM glucose, 1% of penicillin and streptomycin (Gibco) in L-15 supplemented with 1% N2 supplement (Gibco) and 15 μg/ml chicken muscle extract (3.5 mg/ml). 30,000 cells were plated on poly-ornithine/collagen coated cover slips in the presence or absence of the peptide coupled to BSA and incubated in a humidified chamber at 37° C. and 5% CO₂. The length and number of neurites were measured and counted for isolated neurons that were not in contact with other cells and with at least one process that was as long as the diameter of the cell body after 24 hours of culture.

Preparation and Culture of Dorsal Root Ganglion Neurons

The cover slips were prepared identically as for the experiments with motor neurons. Dorsal root ganglia neurons were isolated from embryonic-day 11 chicken eggs. The ganglia were transferred into 1 ml of digestion solution (0.05% Trypsin, 0.01% DNAse 1 in HBSS medium) and incubated 15 min. at 37° C. with resuspending every 2-5 min. The ganglia were then dissociated in 1 ml of ice cold dissociation solution (0.05% DNAse 1, 0.1% BSA, in L15 medium), loaded on 3 ml of a 4% BSA cushion in a 15 ml Falcon tube and centrifuged at 4° C., 600×g for 20 min. The cells were resuspended in 0.5 ml of the complete medium described in the previous section. 20,000 cells were added to wells containing one cover slip, and allowed to grow for 18 hrs in a humidified chamber at 37° C. and 5% CO₂. Fixing and analysis of neurite outgrowth was performed as described in the preceding section.

Immunohistology and Immunocytology

Immunohistology

Cryosections of femoral nerve from a 4-month-old mouse were used to look for binding of peptide-BSA complex. The sections were treated for 1 hr with 1% H₂O₂, 0.5% bovine serum albumin (BSA), and 10% goat serum in PBS, in order to reduce the endogenous peroxidase activity. The sections were then incubated overnight at 4° C. with peptide-BSA complex or BSA (1 mg/ml in PBS, 150 μl cover slips), and then washed 4 times with PBS-0.01% Tween 20. For detection, anti-BSA antibody (Sigma, 1:16 dilution, 150 μl/cover slips) was added and incubated overnight at 4° C. HRP-coupled goat anti rabbit serum was added (1:2000), for 1 hr in a volume of 150 μl per cover slip. The color reaction was developed using a 5% dilution of a 4 mg/ml stock solution of 9-amino-3-ethylcarbazol (AEC, Fluka) in N,N′-dimethylformramide in 0.1 M sodium acetate buffer, pH 4.8, containing 0.1% H₂O₂.L2-412 antibody and HRP-coupled goat anti-rat antibody were used for the positive control. A similar experiment was performed using biotinylated BSA-peptide conjugate. A concentration of 50 μg/ml was used for the overnight incubation and HRP-coupled streptavidin (1:2000) was added for 1 hr. The color reaction was developed as described above.

Immunocytology

Cover slips were coated with polyornithine (1.5 μg/ml) then with collagen (20 μg/ml,) and 40,000 cells were allowed to grow for 40 hrs at 37° C. under 5% CO₂ as described above. The fixed cover slips were then blocked in 5% non-fat dry milk powder in PBS for 2 hrs. After extensive washing with PBS-0.05% Tween-20, biotinylated BSA-peptide conjugate was added at a concentration of 50 μg/ml for 4 hrs. After another six times wash steps, detection was done using HRP-coupled streptavidin, 1:500, for 1 hr. Color detection was as described above for immunohistology. The fixed neurons were photographed at 40× magnification. The images presented were processed for enhanced color rendition using Adobe Photoshop.

### EXAMPLE 10

### Treatment of Oligodendrocyte Cultures with HIV gp120 and L2/HNK-1 Carbohydrate Epitope Mimic Peptide

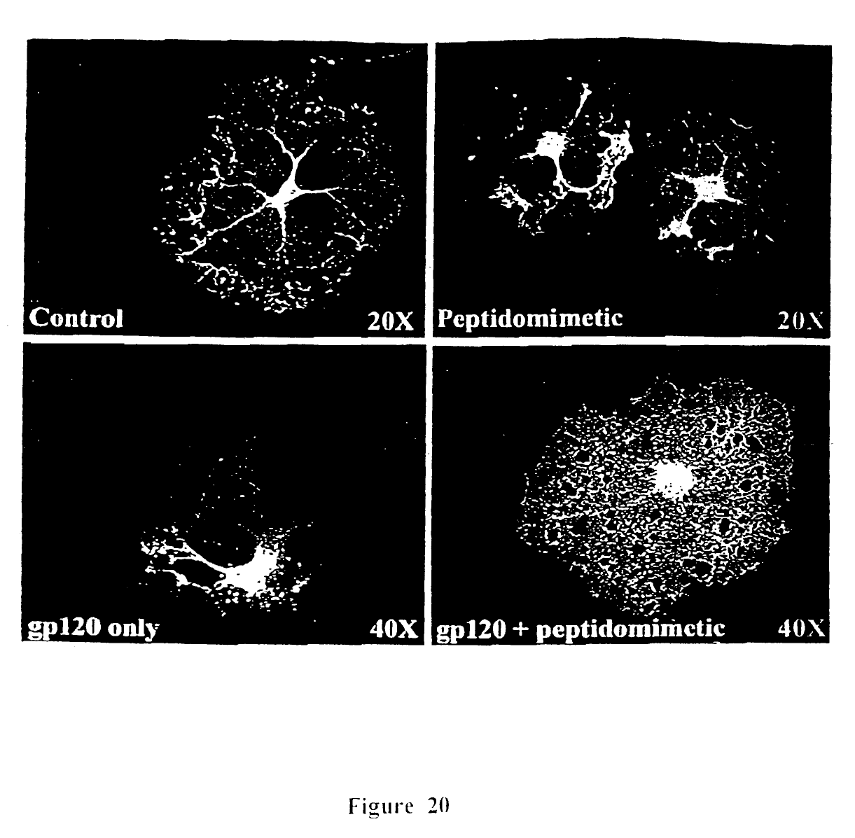

For these experiments, mixed neural cultures were isolated from rat cerebellum. Briefly, tissue was harvested from postnatal day five rat pups, dissociated and plated on poly-lysine coated 24 well cluster plates in Neural basal medium plus B27 supplement, 1% FBS and penicillin and streptomycin. HNK-1 epitope mimic peptide 8 mer (FLHTRLFV) (SEQ ID NO:8) (1 mM, 100 nM or 10 nM) was added to wells at the time of plating. Three days later, 1 nM gp120 was preincubated with 1 mM, 100 nM or 10 nM HNK-1 peptidomimetic for 1 hour at 37° C. After one hour, gp120 or gp120 plus HNK-1 peptidomimetic was added to mixed cerebellar cultures in sets of 6 replicates. Four days later, cultures were fixed and immunostained with the oligodendrocyte specific antibody marker, RIP. Data was analyzed by counting the number of RIP positive cells in twenty 20×-microscope fields. In addition, the number of RIP positive cells, mature oligodendrocytes with extensive intact membrane sheaths were counted. The data is presented in TABLE 6 below. The cells are depicted in FIG. 20. HNK-1 carbohydrate epitope mimic peptide increases the number of mature oligodendrocytes and blocks myelin destruction associated with gp 120 treatment.

TABLE 6

| | # mature RIP + | Standard | Total # RIP + | Standard |
|---|---|---|---|---|
| | cells/20 fields | Deviation | cells/20 fields | Deviation |
| control | 7.333333 | 3.14 | 65.5 | 18.14111 |
| 1 uM | 13.33333 | 3.14 | 62.66667 | 19.24231 |
| peptide | | | | |
| 100 nM | 16.33333 | 3.55 | 76.66667 | 18.2939 |
| peptide | | | | |
| 10 uM | 22.33333 | 8.52 | 75.83333 | 21.22656 |
| peptide | | | | |
| gp120 | 1.666667 | 1.63 | 68.5 | 17.69463 |
| gp120 + | 12.33333 | 3.82 | 76.83333 | 15.72789 |
| 1 uM | | | | |
| peptide | | | | |
| gp120 + | 11.16667 | 3.06 | 69.83333 | 22.26582 |
| 100 nM | | | | |
| peptide | | | | |
| gp120 + | 8.833333 | 2.041 | 73.83333 | 20.90375 |
| 10 nM | | | | |
| peptide | | | | |

### EXAMPLE 11

### L2/HNK-1 Carbohydrate Mimic Peptide Blocks HIV gp120-Mediated Inflammation and Neuropathy

A role has been demonstrated for gp120 binding to peripheral nerve in inducing painful neuropathies. Herzberg et al coated the sciatic nerve of rats with oxidized cellulose saturated with gp120 or BSA as control. Persistant hyperalgesia and allodynia were observed throughout a one month testing period in rats treated with gp120. Thus, it was suggested that binding of gp120 to the peripheral nerve trunk alone can result in persistant painful neuropathy mediated by long term changes in the CNS. This system was utilized to assess the affect of the 8-mer peptide on gp120-mediated inflammation and neuropathy, using Ox42 (MAC 1), TNF and GFAP expression as immunohistochemical markers.

Ox42 (also called MACl) is a general marker for immune/inflammatory activation of the central nervous system (expressed by microglia—when those proliferate and/or extend their processes). If Ox42 marker is present two weeks, and particularly four weeks, after initial neural injury, this indicates a chronic problem. An ongoing inflammatory process in the CNS is correlative of neuropathy. TNF is a cytokine indicating an inflammatory process, and in this case neuronal degeneration. It's expression increases in the CSF of pateints with HIV related neuropathy and is thought to play a role in the degenerative process in these patients (see “role of immune activation and cytokine expression in HIV-1 associated neurologic disease” bay Masaru Yoshioka Walter G Bradley, Paul Shapshak, Isao Nagano, Rene V. Stewart, Ke-Qin Xin, Ashok Srivastava, and Shozo Nakamura in Advance in Neuroimmunology) Vol.5 pp 335-358, 1995. GFAP is a marker for a general activation of astrocytes in the spinal cord and is expressed following damage to the CNS.

The amount of Ox42, TNF and GFAP staining was quantitated and is presented in

TABLE 7

| Marker | gp120 alone | gp120 + peptide | Oxycel alone | Background |
|---|---|---|---|---|
| Ox42 | 37556 | 1557 | 1678 | 1289 |
| SE | 598 | 107 | 78 | 55 |
| TNF | 2487 | 1478 | 1248 | 789 |
| SE | 154 | 98 | 38 | 83 |
| GFAP | 1245 | 8745 | 6787 | 3875 |
| SE | 104 | 93 | 55 | 257 |

Background represents no sciatic manipulation—historical data from previous experiments. Values are expressed as immunoreactive area, in pixels.

As indicated in TABLE 7, gp120 alone consistently induces higher values of immunoreactivity compared with Oxycel alone when applied to the sciatic nerve. The HNK-1 epitope mimic peptide brings those values back to the same level as with Oxycel alone.

On review of the cellular morphology under light microscopy of the spinal cord sections, blebbing of the cell membranes and shrinkage of the nucleus, indicative morphologically of apopotosis, is observed on treatment of gp120 alone. Cell death is correlative with CNS neuropathy and degenerative processes. This cellular morphology was not observed in the animals treated with gp120 in combination with the HNK-1 peptide.

Methods

Male Sprague Dawley rats weighing 200-225 g were used.

The left sciatic nerve was isolated by blunt dissection, and wrapped with oxidized cellulose (Oxycel, Becton Dickinson) saturated with 250 ul sterile saline containing 400 ng of recombinant gp120 protein alone, or in the presence of 43 ng of the 8-mer L2/HNK-1 mimic peptide (corresponding to a 10×excess of gp120 on a mole-per-mole basis), utilizing the Oxycel delivery method of Eliav et al (Eliav et al (1999) Pain 83(2): 169-82. Eight animals were treated with gp120 alone and eight animals were treated with gp120 and 43 ng peptidomimetic compound.

Animals were euthanized (overdose of xylazine/ketamine) at two and four weeks following surgery and perfused transcardially with ice cold saline followed by 4% paraformaldehyde.

Spinal cords were harvested and cryoprotected overnight at 30% sucrose solution 10 Sections of 40 um thickness from the lumbar enlargement from each animal were thaw mounted onto gelatized slides and stained immunohistochemically for Ox-42 (MACl), TNF and GFAP.

Using the NIH image analysis software package the immunoreactive area was quantified and is presented in TABLE 7.

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

The following is an alphabetical list of the references referred to herein. The disclosures of the listed references as well as other publications, patent disclosures or documents recited herein, are all incorporated herein by reference in their entireties.

Abo, T. and Balch, C. M., J. Immunol. 127, 1024-1029 (1981).

Agadjanyan, M., et al., Nature Biotechnology 15, 547-551 (1997).

Apostolopoulos, V., et al., Nature Biotechnology 16, 276-280 (1998).

Arakawa, Y., et al., J. Neuroscience 10, 3507-3515 (1990).

Bakker, H., et al., J. Biol. Chem. 272, 29942-29946 (1997).

Bonnycastle, L., et al., J. Mol. Biol. 258, 747-762 (1996).

Bradford, M. Analytical Biochemistry 72, 248-254 (1976).

Bronner-Fraser, M. Dev. Biol. 123, 321-331 (1987).

Brushart, T. Restorative Neurology and Neuroscience 1, 281-287 (1990).

### Burger, D., et al., J. Neurochem. 58, 854-61 (1992).

Burger, D., et at., J. Neuochem. 54, 1569-75 (1990).

Burger, D., et al., J. Neurochem. 61, 1822-27 (1993).

Carlson, J., Drevin, et al., Biochem. J. 173, 723-737 (1978).

Chada, S., et al., J. Cell Science 110, 1179-1186 (1997).

Chou, D., et al., J. Biol. Chem. 266, 17941-17947 (1991).

Chou, D., and Jungalwala, F. J. Neurochem. 62, 307-314 (1994).

Chou, D., and Jungalwala, F. J. Biol. Chem. 271, 28868-28874 (1996).

Chou, D., and Jungalwala, F. J. Biol. Chem. 268, 21727-21733 (1993).

Chou, D. K., et al., J. Biol. Chem. 261, 11717-25 (1986).

Chou, D. K. H., et al., J. Neurochem. 57, 852-859 (1991).

Chou, K. H., et al., Biochem. Biophys. Res. Commun. 128, 383-388 (1985).

Cortese, R., et al., Current Opinion in Biotechnology 6, 73-80 (1995).

Cwirla, S. E. et al., Proc. Natl. Acad. Sci. USA, 87, 6378-6382 (1990).

Daniels, D. A., and Lane, D. P. Methods 9, 494-507 (1996).

Deng, S. -J. et al., J. Biol. Chem. 269, 9533-9538 (1994).

Dennis, R. D., et al., J. Neurochem. 51, 1490-1496 (1988).

Dennis, RD., et al., Cell Tissue Res., 265, 589-600 (1991).

Devlin, J. J. et al., Science 249, 404-406 (1990).

Dieperink, M., et al., J. Neuroscience 12, 2177-2185 (1992).

Dorries, U., et al., J. Neuroscience Res. 43, 420-438 (1996).

Faissner, A. Neuroscience Letters 83, 327-332 (1987).

Faissner, A. Cell Tissue Research 290, 331-341 (1997).

Fawcett, J. Trends in Neurosci. 21 (5), 179-180 (1998).

Filbin, M. T., and Tennekoon, G. H. J. Cell. Biol. 122, 451-459 (1993).

Giese, K. P. et al., Cell 71, 565-576 (1992).

Griffith, L. S., et al., J. Neuroscience Res. 33, 639-48 (1992).

Griffiths, A., et al., EMBO J. 13, 3245-3260 (1994).

Hall, H. et al., Eur. J. Neurosci. 5, 34-42 (1993).

Hall, H., et al., J. Neurochem. 68, 544-553 (1997a).

Hall, H., et al., Eur. J. Biochem. 246, 233-242 (1997b).

Hall, H., et al., Glycobiology 5, 435-441 (1995).

Hoess, R., et al., Gene 128, 43-49 (1993).

Hynes, R. O. Cell 48, 549-554 (1987).

Ilyas, A. A. et al., Brain Res. 385, 1-9 (1986).

Ilyas, A. A. et al. Proc. Natl. Sci. USA 81, 1225-9 (1984).

Ilyas, A. A., et al. J. Neurochem. 55, 594-601 (1990).

Kanda, T., et al., Proc. Natl. Acad. Sci. USA 92, 7897-7901 (1995).

Kapfhammer, J. P. Anat. Embryol. 196, 417-426 (1997).

Kay, K. B. et al., Gene 128, 59-65 (1993).

Kieber-Emmons, T. Immunologic Research 17, 95-108 (1998).

Keilhauer, G., et al., Nature 316, 728-730 (1985).

Kruegger, R. C., et al., J. Biol. Chem. 267, 12149-12161 (1992).

Kruse, J., et al. Nature 316, 146-148 (1985).

Künemund, V., Jungalwala, F. B., J. Cell Biol. 106, 213-223 (1988).

Kruse, J. et al., Nature 311, 153-155 (1984).

Lafont, F., et al., Dev. Biol. 165, 453-468 (1994).

Lasonder, E. et al., Nucl. Acids Res. 22, 545-546 (1994).

Le Roux, P., and Reh, T. J. Neuroscience 14, 4639-4655 (1994).

Lein, P., and Higgins, D. Dev. Biol. 136, 330-345 (1989).

Lein, P., et al., Neuron 15, 597-605 (1995).

Levi, A., et al., R. J. Neuroscience 14, 1309-1319 (1994).

Low, K., et al., Eur. J. Neurosci. 6, 1773-81 (1994).

Lyons, L., and Zinder, N. Virology 49, 45-60 (1972).

Magliani, W., et al., Nature Medicine 4, 705-709 (1998).

Martini, R. J. Neurocytol. 23, 1-28 (1994).

Martini, R., et al., Devel. Biol. 129, 330-338 (1988).

Martini, R., et al., J. Neurosci. 14, 7180-7191 (1994).

Martini, R. et al., Eur. J. Neurosci. 4, 628-639 (1992).

McGarry, R. C., et al., Nature 306, 376-378 (1983).

Metcalfe, W. K. et al., Development 110, 491-504 (1990).

Mikol, D. D., et al., J. Cell Biol. 110, 471-9 (1990).

Montag, D. et al., Neuron 13, 229-246 (1994).

Nair, S., et al., J. Comp. Neurol. 332, 282-292 (1993).

Nair, S. M., and Jungalwala, F. B. J. Neurochem. 68, 1286-1297 (1997).

Nakano, T., et al., Tetrahedron Letters, 32, 1569-1572 (1991).

Needham, L. K. and Schnaar, R. L. Proc. Natl. Acad. Sci. USA 90, 1359-63 (1993).

Newgreen, D. F., and Hartley, L. Acta Anat. 154, 243-260 (1995).

Norohna, A. et al., Brain Res. 385, 237-244 (1986).

Oldenburg, R. K. et al., Proc. Natl Acad Sci. 89, 5393-5397 (1992).

Oka, S., et al., J. Biol. Chem. 267, 22711-22714 (1992).

Ong. E., et al., J. Biol. Chem. 273, 5190-5195 (1998).

Pesheva, P., et al., Neurosci. Lett. 83, 303-306 (1987).

Phalipon, A., et al., Eur. J. Immunol. 27, 2620-2625 (1997).

Poltorack, M., et al., J. Cell. Biol. (1987)

Prochiantz, A. Neuron 15, 743-746 (1995).

Quarles, R. H., et al., Biology and Chemistry, 4413-4448 (1992).

Quarles, R. H. J. Molecular Neuroscience 8, 1-12 (1997).

Schachner, M. Ciba Fdn. Symp. 145, 56-172 (1989).

Schatz, P., and Scott, J. Current Opinion in Biotechnology 5, 487-494 (1994).

Schmitz, B., et al., Glycoconj. J 11, 345-52 (1994).

Schuller-Petrovic, S., et al., Nature 306, 179-181 (1983).

Schwarting, G. A., Devel. Biol. 120, 65-76 (1987).

Scott, J. K., and G. P. Smith Science 249, 386-390 (1990).

Scott, J. K., et al., Proc. Natl. Acad. Sci. USA 89, 5398-5402.

Smith, G. Lab Protocols (1992).

Smith, G. P., and Scott, J. K. Methods Enzymol. 217, 228-257 (1993).

Snipes, G. J., et al. J. Neurochem. 61, 1961-1964 (1993).

Suter, U., and Snipes, G. J. J. Neuroscience Res. 40, 145-151 (1995).

Schwab, M., Bartholdi, D. Physiological Reviews, 319-37 (1996).

Taki, T., et al., FEBS Letters 418, 219-223 (1997).

Tatum, A. H. Ann. Neurol. 33, 502-6 (1993).

Terayarna, K., et al., Proc. Natl. Acad. Sci. USA 94, 6093-6098 (1997).

Vogel, M., et al., Biochem. J. 278, 199-202 (1991).

Voshol, H., et al., J. Biol. Chem. 271, 22957-22960 (1996).

Westerink, J., Giardina, P., Apicella, M., and Kieber-Emmons, T. (1995). Peptide mimicry of the meingococcal group C capsular polysaccharide. Proc. Natl. Acad. Sci. USA. 92.

Xiao, Z., Taylor, J., Montag, D., Rougon, G., and Schachner, M. (1996). Distinct effects of recombinant tenascin-R domains in neuronal cell functions and identification of the domain interacting with the neuronal recognition molecule F3/F 11. Eur. J. Neuroscience 8, 766-782

Yamawaki, M., Ariga, T., Bigbee, J., Ozawa, H., Kawashima, I., Tai, T., Kanda, T., and YU, R. (1996). Generation and Characterization of Anti-Sulfoglucuronyl Paragloboside Monoclonal Antibody NGR50 and its Immunoreactivity With Peripheral Nerve. Journal of Neuroscience Research 44, 586-593.

Yoshihara, Y., Oka, S., Nemoto, Y., Watanable, Y., Nagata, S., Kagamiyama, H., and Mori, K. (1994). An ICAM-Related Neuronal Glycoprotein, Telencephalin, with Brain Segment-Specific Expression. Neuron 12, 541-553.

Yoshihara, Y., Oka, S., Watanabe, Y., and Mori, K. (1991). Developmental and Spatially Regulated Expression of HNK-1 Carbohydrate Antigen on a Novel Phosphatidylinositol-anchored Glycoprotein in Rat Brain. The Journal of Cell Biology 1115, 731-744.

Yu, J., and Smith, G. (1996). Affinity Maturation of Phage-Displayed Peptide Ligands. Methods in Enzymology 267, 3-27.

What is claimed is:

1. An isolated peptide which mimics the carbohydrate epitope GlcAβ1→3Galβ1→4GlcNAc or or sulfate -3GlcAβ1→3Galβ1→4GlcNAc.

2. An isolated peptide comprising an amino acid sequence X₁ X₂ X₃ X₄ X₅ L/V X₆ X₇ X₈ X₉ X₁₀ X₁₁ X₁₂ X₁₃ X₁₄, wherein each residue can be independently selected as follows (SEQ ID NO: 1):
X₁ is T, S, A or P;
X₂ is L, I, V, M, F, H, W or N;
X₃ is T, S, A, H, Y, F, W, N, D or E;
X₄ is R, Q, K, T, S or A;
X₅ is V, I, L, M, R, Q or K;
X₆ is T, S, A, Y, F, H, W, N, L, I, V or M;
X₇ is D, E, V, L, I, M, F, Y, K W or N;
X₈ is V, I, L, M, S, A, T, R, Q or K;
X₉ is Y, F, H, W, D, E, I, V, L, M or N;
X₁₀ is R, Q, K, W, Y, F, H, N, V, I, L, M or G;
X₁₁ is G, Y, F, H, W, N, S, A, T, I, V, L, M;
X₁₂ is R, Q, K, H, N, Y, F, W, I, V, L or M;
X₁₃ is L, V, I, M, T, S or A; and
X₁₄ is S, T, A, P, G, R, Q or K;
and variants, analogs and active fragments thereof.

3. An isolated peptide comprising an amino acid sequence F L H T R L X₁ X₂ X₃ X₄ X₅ X₆ X₇ X₈ X₉, wherein each residue can be independently selected as follows (SEQ ID NO: 2):
X₁ is T, S, A, Y, F, H, W, N, L, I, V or M;
X₂ is D, E, V, L, I, M, F, Y, H, W or N;
X₃ is V, I, L, M, S, A, T, R, Q or K;
X₄ is Y, F, H, W, D, E, I, V, L, M or N;
X₅ is R, Q, K, W, Y, F, H, N, V, I, L, M or G;
X₆ is G, Y, F, H, W, N, S, A, T, I, V, L, M;
X₇ is R, Q, K, H, N, Y, F, W, I, V, L or M;
X₈ is L, V, I, M, T, S or A; and
X₉ is S, T, A, P, G, R, Q or K;
and variants, analogs and active fragments thereof.

4. An isolated peptide comprising an amino acid sequence F L H T R L F V X₁ X₂ X₃ X₄ X₅ X₆ X₇, wherein each residue can be independently selected as follows (SEQ ID NO: 3):
X₁ is V, I, L, M, S, A, T, R, Q or K;
X₂ is Y, F, H, W, D, E, I, V, L, M or N;
X₃ is R, Q, K, W, Y, F, H, N, V, I, L, M or G;
X₄ is G, Y, F, H, W, N, S, A, T, I, V, L, M;
X₅ is R, Q, K, H, N, Y, F, W, I, V, L or M;
X₆ is L, V, I, M, T, S or A; and
X₇ is S, T, A, P, G, R, Q or K;
and variants, analogs and active fragments thereof.

5. An isolated peptide comprising the amino acid sequence F L H T R L F V S D W Y H T (SEQ ID NO: 7).

6. An isolated peptide comprising the amino acid sequence F L H T R L F V (SEQ ID NO: 8).

7. An isolated peptide comprising the amino acid sequence TRLFR(V/F) (SEQ ID NO: 39).

8. An isolated peptide comprising the amino acid sequence TRLF(R)V (SEQ ID NO: 40).

9. An isolated peptide comprising the amino acid sequence TRLF (SEQ ID NO: 41).

10. An isolated peptide having the amino acid sequence set out in any of SEQ ID NO: 27-38.

11. A method for promoting neural growth and/or remyelination and/or neuroprotection in vivo in the central nervous system of a mammal comprising administering to said mammal a neural growth and/or remyelination and/or neuroprotection promoting amount of the peptide of claim 1, which molecule is capable of overcoming inhibitory molecular cues found on glial cells and myelin and promoting said neural growth, active fragments thereof, cognates thereof, congeners thereof, mimics thereof, antagonists thereof, antibodies thereto, analogs thereof, secreting cells thereof and soluble molecules thereof.

12. The method of claim 11 further comprising administering to said mammal a neural growth and/or remyelination and/or neuroprotection promoting amount of a neural cell adhesion molecule.

13. The method of claim 12 wherein said neural cell adhesion molecule is selected from the group consisting of L1, N-CAM and myelin-associated glycoprotein, laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

14. A method for promoting neural growth and/or remyelination and/or neuroprotection in vivo in the central nervous system of a mammal comprising administering to said mammal a neural growth promoting amount of an agent, said agent comprising a neural cell adhesion molecule, which molecule is capable of overcoming inhibitory molecular cues found on glial cells and myelin and promoting said neural growth, active fragments thereof, secreting cells thereof and soluble molecules thereof, said agent being modified by recombinant or chemical means to have the peptide of any of claim 1 attached thereto.

15. The method of claim 14 wherein said neural cell adhesion molecule is selected from the group consisting of L1, N-CAM and myelin-associated glycoprotein, laminin, fibronectin, N-cadherin, BSP-2/D2 (mouse N-CAM), 224-1A6-A1, L1-CAM, NILE (rat L1), Nr-CAM, TAG-1 (axonin-1), Ng-CAM and F3/F11/contactin.

16. A method for enhancing memory, comprising administering to the brain of a mammal in need of such enhancement, an amount of the peptide of claim 1 effective to enhance the memory of the mammal.

17. A method of claim 16 which further comprises administering to the brain of said mammal an amount of a neural cell adhesion molecule effective to enhance the memory of the mammal.

18. A method for enhancing memory, comprising delivering to the cells of the brain of a mammal in need of such enhancement, a vector which allows for the expression of the peptide of any of claim 1.

19. The method for enhancing memory in accordance with any of claims 12 or 14, which comprises a method for inhibiting the onset or progression, or treating the presence or consequences of Alzheimers disease or dementia in a mammal.

20. A method for increasing synaptic efficacy in the CNS of a mammal comprising administering to the brain of the mammal, an amount of the peptide of claim 1 effective to increase synaptic efficacy in the brain of the mammal.

21. The method of claim 20, wherein the increase in synaptic efficacy is demonstrated by the stabilization of long term potentiation.

22. A method of promoting neuroprotection and/or neuronal survival in a mammal comprising delivering to the cells of the brain of a mammal in need thereof, a vector which allows for the expression of the peptide of claim 1.

23. The method of claim 22 which comprises a method for inhibiting the development or onset, or treating the presence in a mammal of a condition selected from the group consisting of apoptosis, necrosis, Alzheimers disease, dementia, Parkinsons disease, multiple sclerosis, acute spinal cord injury, chronic spinal cord injury, any of the foregoing where neurodegeneration occurs or may occur, and combinations thereof.

24. A method for inhibiting axonal cell death and enhancing myelination and remyelination in the central nervous system of a mammal comprising administering to said mammal a therapeutically effective amount of a peptide of claim 1, which peptide is capable of overcoming inhibitory molecular cues found on glial cells and myelin and promoting said neural growth, active fragments thereof, cognates thereof, congeners thereof, mimics thereof, antagonists thereof, antibodies thereto, analogs thereof, secreting cells thereof and soluble molecules thereof.

25. A pharmaceutical composition for the modulation of neural growth in the central nervous system of a mammal, comprising a therapeutically effective amount of a peptide of claim 1, which peptide is capable of overcoming inhibitory molecular cues found on glial cells and myelin and promoting said neural growth, variants, analogs, active fragments thereof, and secreting or expressing cells thereof, and a pharmaceutically acceptable carrier.

26. A derivative of the peptide of claim 1, capable of mimicking the carbohydrate epitope GlcAβ1→3Galβ1→4GlcNAc or sulfate -3GlcAβ1→3Galβ1→4GlcNAc, having one or more chemical moieties attached thereto.

27. The derivative of claim 26, wherein at least one of said chemical moieties is a water-soluble polymer capable of enhancing solubility of said peptide.

28. The derivative of claim 26, wherein at least one of said chemical moeities is a molecule which facilitates transfer or transport across the blood brain barrier.

29. The derivative of claim 28, wherein said molecule is selected from the group consisting of a biocompatible hydrophobic molecule, transferrin, ApoE or ApoJ.

30. The derivative of claim 26, wherein at least one of said chemical moieties is a molecule having multiple sites for peptide attachment and capable of binding at least two of said peptides simultaneously to generate a multimeric peptide structure.

31. The derivative of claim 30 where said molecule is selected from the group of BSA, ovalbumin, human serum albumin, polyacrylamide, beads and synthetic fibers (biodegradable and non-biodegradable).

32. The derivative of claim 26, wherein at least one of said chemical moieties is a neural cell adhesion molecule.

33. The derivative of claim 26, wherein at least one of said chemical moieties is a branched or unbranched polymer.

34. The derivative of claim 26, wherein at least one of said chemical moieties is N-terminally attached to said peptide.

35. The derivative of claim 26, wherein at least one of said chemical moieties is C-terminally attached to said peptide.

36. A DNA sequence which encodes a peptide of claim 1.

37. A DNA sequence which encodes a peptide of claim 1, or a fragment thereof, selected from the group consisting of:
(A) DNA capable of encoding the peptide set out in any of SEQ ID NOS: 1-8 and 27-41;
(B) DNA sequences that hybridize to any of the foregoing DNA sequences under standard hybridization conditions; and
(C) DNA sequences that code on expression for an amino acid sequence encoded by any of the foregoing DNA sequences.

38. A recombinant DNA molecule comprising a DNA sequence or degenerate variant thereof and a heterologous nucleotide sequence, wherein said DNA sequence or degenerate variant encodes a peptide of claim 1, or a fragment thereof, selected from the group consisting of:
(A) DNA capable of encoding the peptide set out in any of SEQ ID NOS: 1-8 and 27-41;
(B) DNA sequences that hybridize to any of the foregoing DNA sequences under standard hybridization conditions; and
(C) DNA sequences that code on expression for an amino acid sequence encoded by any of the foregoing DNA sequences.

39. The recombinant DNA molecule of claim 38, wherein said DNA sequence is operatively linked to an expression control sequence.

40. The recombinant DNA molecule of claim 38, wherein said expression control sequence is selected from the group consisting of the early or late promoters of SV40 or adenovirus, the lac system, the trp system, the TAC system, the TRC system, the major operator and promoter regions of phage λ, the control regions of fd coat protein, the promoter for 3-phosphoglycerate kinase, the promoters of acid phosphatase and the promoters of the yeast α-mating factors the promoters of neural cell adhesion molecules, the promoter of L1, the gFAP promoter, and the promoter for myelin basic protein.

41. A unicellular host transformed with a recombinant DNA molecule comprising a DNA sequence or degenerate variant thereof, which encodes a peptide of claim 1, or a fragment thereof, selected from the group consisting of:
(A) DNA capable of encoding the peptide set out in any of SEQ ID NOS: 1-8 and 27-41;
(B) DNA sequences that hybridize to any of the foregoing DNA sequences under standard hybridization conditions; and
(C) DNA sequences that code on expression for an amino acid sequence encoded by any of the foregoing DNA sequences;
wherein said DNA sequence is operatively linked to an expression control sequence.

42. The unicellular host of claim 41 wherein the unicellular host is selected from the group consisting of E. coli, Pseudomonas, Bacillus, Streptomyces, yeasts, CHO, R1.1, B-W, L-M, COS 1, COS 7, BSC1, BSC40, and BMT10 cells, plant cells, insect cells, mammalian cells, human cells and neural cells in tissue culture.

43. A cloning vector which comprises the DNA sequence according to claim 36 and a heterologous nucleotide sequence.

44. An expression vector which comprises the DNA sequence according to claim 36 and a heterologous nucleotide sequence.

45. The expression vector of claim 44 wherein the heterologous nucleotide sequence is an expression control sequence.

46. The expression vector of claim 44 wherein the heterologous nucleotide sequence encodes a neural cell adhesion molecule.

47. A method for detecting the presence or activity of a peptide or compound, said peptide or compound capable of mimicking the carbohydrate epitope GlcAβ1→3Galβ1→4GlcNAc or sulfate -3GlcAβ1→3Galβ1→4GlcNAc wherein said peptide or compound is measured by:
A. contacting a sample in which the presence or activity of said peptide or compound is suspected with a binding partner of said peptide or compound under conditions that allow binding of said peptide or compound to said binding partner to occur; and
B. detecting whether binding has occurred between said peptide or compound from said sample and the binding partner;
wherein the detection of binding indicates that presence or activity of said peptide or compound in said sample.

48. The method of claim 47 wherein the binding partner is selected from the group consisting of an antibody which recognizes GlcAβ1→Galβ1→4GlcNAc; an antibody which recognizes sulfate -3GlcAβ1→3Galβ1→4GlcNAc; L2-412 antibody; HNK-1antibody; a polypeptide molecule which binds or otherwise interacts with GlcAβ1→3Galβ1→4GlcNAc or sulfate -3GlcAβ1→3Galβ1→4GlcNAc; laminin; P-selectin; L-selectin; and a neural cell adhesion molecule.

49. A method of testing the ability of a drug or other entity to mimic the carbohydrate epitope GlcAβ1→3Galβ1→4GlcNAc or sulfate -3GlcAβ1→3Galβ1→4GlcNAc which comprises:
a. adding CNS neurons to a cell culture system;
b. adding the drug or other entity under test to the cell culture system;
c. measuring the neuronal outgrowth of the CNS neurons; and
d. correlating a difference in the level of neuronal outgrowth of cells in the presence of the drug relative to a control culture to which no drug is added to the ability of the drug to mimic the carbohydrate epitope GlcAβ1 →3Galβ1→4GlcNAc or sulfate -3GlcAβ1→3Galβ1→4GlcNAc.

50. A test kit for the demonstration of a molecule capable of binding GlcAβ1→3Galβ1→4GlcNAc or sulfate -3GlcAβ1→3Galβ1→4GlcNAc in a eukaryotic cellular sample, comprising:
A. a predetermined amount of a detectably labeled compound or peptide, said peptide or compound capable of mimicking the carbohydrate epitope GlcAβ1→3Galβ1→4GlcNAc or sulfate -3GlcAβ1→3Galβ1→4GlcNAc;
B. other reagents; and
C. directions for use of said kit.

51. A test kit for demonstrating the presence of a molecule capable of binding GlcAβ1→3Galβ1→4GlcNAc or sulfate -3GlcAβ1→3Galβ1→4GlcNAc in a eukaryotic cellular sample, comprising:
A. a predetermined amount of a compound or peptide, said peptide or compound capable of mimicking the carbohydrate epitope GlcAβ1→3Galβ1→4GlcNAc or sulfate -3GlcAβ1→3Galβ1→4GlcNAc;
B. a predetermined amount of a specific binding partner of said compound or peptide;
C. other reagents; and
D. directions for use of said kit;
wherein either said compound or peptide or said specific binding partner are detectably labeled.

52. A pharmaceutical composition for promoting neural growth and/or remyelination and/or neuroprotection, comprising a therapeutically effective amount of the peptide of claim 1 or variants or analogs thereof and a pharmaceutically acceptable carrier.

53. The pharmaceutical composition of claim 52 further comprising a therapeutically effective amount of a neural cell adhesion molecule.

54. A method for preventing, ameliorating or blocking viral infection of a mammal comprising administering to said mammal an effective amount of the peptide of claim 1, variants thereof, analogs thereof, active fragments thereof or derivatives thereof.

55. The method of claim 54 wherein the viral infection is the result of the human immunodeficiency virus.

56. A method for preventing, ameliorating or blocking neuropathy in a mammal comprising administering to said mammal an effective amount of the peptide of claim 1, variants thereof, analogs thereof, active fragments thereof or derivatives thereof, wherein said neuropathy is viral-mediated, immune-mediated or the result of trauma.

57. A pharmaceutical composition for preventing, ameliorating or blocking viral infection comprising a therapeutically effective amount of the peptide of claim 1 or variants, analogs, derivatives or active fragments thereof and a pharmaceutically acceptable carrier.
